(19) United States
(12) Patent Application Publication
(10) Pub. No.: US 2019/0376971 A1
(43) Pub. Date: Dec. 12, 2019

(54) Compositions and Methods for the Assessment of Drug Target Occupancy for Bruton's Tyrosine Kinase

(71) Applicant: Acerta Pharma B.V., Oss (NL)

(72) Inventors: Tjeerd Barf, Ravenstein (NL); Allard Kaptein, Zaltbommel (NL); Saskia Verkaik, Macharen (NL); Dennis Demont, Oss (NL); Todd Covey, San Carlos, CA (US); Bas Van De Kar, Oss (NL); Bart Van Lith, Uden (NL); Michael Gulrajani, San Carlos, CA (US)

(21) Appl. No.: 16/477,395

(22) PCT Filed: Jan. 19, 2018
(86) PCT No.: PCT/IB2018/050364

§ 371 Date: Jul. 11, 2019

Related U.S. Application Data
(60) Provisional application No. 62/448,077, filed on Jan. 19, 2017.

(51) Int. Cl. G01N 33/573; C07D 519/00

(52) U.S. Cl. CPC G01N 33/573; C12Q 1/485; C07D 519/00

(57) ABSTRACT

In some embodiments, the invention relates to compositions, methods, and kits for assessment of drug target occupancy in Bruton's tyrosine kinase (BTK) in a selective and sensitive manner for use with BTK inhibitor therapy in the treatment of Bruton's tyrosine kinase (BTK) mediated disorders, including cancers, inflammatory diseases, and immune and autoimmune diseases.

### FIELD OF THE INVENTION

In some embodiments, the present invention relates to compositions and methods for the assessment of the occupancy of Bruton's tyrosine kinase (BTK), including compositions and methods useful in the treatment of cancers and immune, autoimmune, and inflammatory diseases by BTK inhibitors.

### BACKGROUND OF THE INVENTION

Bruton's tyrosine kinase (BTK) is a Tec family non-receptor protein kinase expressed in B cells and myeloid cells. The function of BTK in signaling pathways activated by the engagement of the B cell receptor (BCR) and FcεR1 on mast cells is well established. In addition, a function for BTK as a downstream target in Toll like receptor signaling is suggested. BTK is composed of the pleckstrin homology (PH), Tec homology (TH), Src homology 3 (SH3), Src homology 2 (SH2), and tyrosine kinase or Src homology 1 (TK or SH1) domains. The function of BTK in signaling pathways activated by the engagement of the B cell receptor (BCR) in mature B cells and FCER1 on mast cells is well established. Functional mutations in BTK in humans result in a primary immunodeficiency disease (X-linked agammaglobulinemia) characterized by a defect in B cell development with a block between pro- and pre-B cell stages. The result is an almost complete absence of B lymphocytes, causing a pronounced reduction of serum immunoglobulin of all classes. These findings support a key role for BTK in the regulation of the production of auto-antibodies in autoimmune diseases.

BTK is expressed in numerous B cell lymphomas and leukemias. Other diseases with an important role for dysfunctional B cells are B cell malignancies, as described in Hendriks, et al., Nat. Rev. Cancer, 2014, 14, 219-231. The reported role for BTK in the regulation of proliferation and apoptosis of B cells indicates the potential for BTK inhibitors in the treatment of B cell lymphomas. BTK inhibitors have thus been developed as potential therapies for many of these malignancies, as described in D'Cruz, et al., OncoTargets and Therapy 2013, 6, 161-176. With the regulatory role reported for BTK in FcεR-mediated mast cell activation, BTK inhibitors may also show potential in the treatment of allergic responses, as described in Gilfillan, et al., Immunologic. Rev. 2009, 288, 149-169. Furthermore, BTK is also reported to be implicated in RANKL-induced osteoclast differentiation, as described in Shinohara, et al., Cell 2008, 132, 794-806, and therefore may also be of interest for the treatment of bone resorption disorders. Other diseases with an important role for dysfunctional B cells are B cell malignancies. The reported role for BTK in the regulation of proliferation and apoptosis of B cells indicates there is potential for BTK inhibitors in the treatment of B cell lymphomas as well. Inhibition of BTK appears to be relevant for diseases such as B cell lymphomas because of chronic active BCR signaling, as described in Davis, et al., Nature, 2010, 463, 88-94.

Most BTK inhibitors reported to date, such as ibrutinib, are not selective over other kinases. With adverse effects reported for knockouts of Src-family kinases, especially for double and triple knockouts, this is seen as a barrier for the development of BTK inhibitors that are not selective over the Src-family kinases. Both Lyn-deficient and Fyn-deficient mice exhibit autoimmunity mimicking the phenotype of human lupus nephritis. In addition, Fyn-deficient mice also show pronounced neurological defects. Lyn knockout mice also show an allergic-like phenotype, indicating Lyn as a broad negative regulator of the IgE-mediated allergic response by controlling mast cell responsiveness and allergy-associated traits, as described in Odom, et al., J. Exp. Med., 2004, 199, 1491-1502. Furthermore, aged Lyn knock-out mice develop severe splenomegaly (myeloid expansion) and disseminated monocyte/macrophage tumors, as described in Harder, et al., Immunity, 2001, 15, 603-615. These observations are in line with hyperresponsive B cells, mast cells and myeloid cells, and increased Ig levels observed in Lyn-deficient mice. Female Src knockout mice are infertile due to reduced follicle development and ovulation, as described in Roby, et al., Endocrine, 2005, 26, 169-176. The double knockouts Src−/−Fyn−/− and Src−/−Yes−/− show a severe phenotype with effects on movement and breathing. The triple knockouts Src−/−Fyn−/−Yes−/− die at day 9.5, as shown by Klinghoffer, et al., EMBO J., 1999, 18, 2459-2471. For the double knockout Src−/−Hck−/−, two thirds of the mice die at birth, with surviving mice developing osteopetrosis, extramedullary hematopoiseis, anemia, leukopenia, as shown by Lowell, et al., Blood, 1996, 87, 1780-1792. Hence, an inhibitor that inhibits multiple or all kinases of the Src-family kinases simultaneously may cause serious adverse effects. More selective BTK inhibitors such as acalabrutinib can avoid these adverse effects from off-target interactions with other kinases.

In the case of covalent irreversible BTK inhibitors, the pharmacodynamic (PD) effect is largely determined by the de novo protein synthesis rate of the target protein (BTK). When full BTK target occupancy is achieved by the drug, further increases in drug levels in the circulation will not affect the target-related efficacy, but may cause off-target binding, potentially increasing adverse events associated with over-dosing. Therefore, there is a need for a selective BTK probe that is also highly sensitive for drug target occupancy measurements in research, clinical, commercial, and preclinical settings.

### SUMMARY OF THE INVENTION

In an embodiment, the invention provides a method for determining a drug target occupancy of Bruton's tyrosine kinase (BTK) in a patient after treatment of the patient with a BTK inhibitor, comprising the steps of:

(a) obtaining a tissue sample from the patient;

(b) separating a population of cells from the tissue sample;

(c) contacting a BTK probe with the population of cells;

(d) detecting the amount of BTK bound to the BTK probe using an assay; and

(e) determining the drug target occupancy of BTK in the population of cells based on the amount of BTK bound to the BTK probe;

wherein the BTK probe is a compound according to:

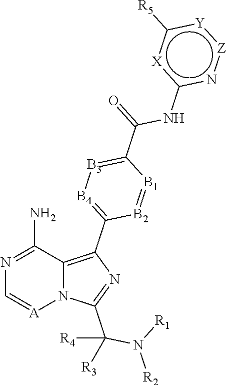

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of heterocycloalkyl and heteroalkyl;
   L₂ is a linear linker group selected from the group consisting of (C₁₋₅)alkylamide, (C₁₋₅)alkoxy, and a bond;
   L₃ is a linear linker group selected from the group consisting of (C₁₋₅)alkylamide, (C₁₋₅)alkoxy, and a bond;
   L₄ is a linear linker group selected from the group consisting of (C₁₋₅)alkylamide, (C₁₋₅)alkoxy, and a bond;
   m is 0 to 5;
   n is 0 to 5; and
   W is:

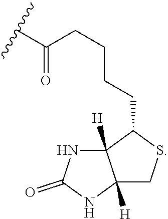 [SMILES: *C(=O)CCCC[C@@H]1SC[C@@H]2NC(=O)N[C@@H]21]

In an embodiment, the invention provides a method for determining a drug target occupancy of Bruton's tyrosine kinase (BTK) in a patient after treatment of the patient with a BTK inhibitor, comprising the steps of:

(a) obtaining a tissue sample from the patient;

(b) separating a population of cells from the tissue sample;

(c) contacting a BTK probe with the population of cells;

(d) detecting the amount of BTK bound to the BTK probe using an assay; and

(e) determining the drug target occupancy of BTK in the population of cells based on the amount of BTK bound to the BTK probe;

wherein the BTK probe is a compound according to:

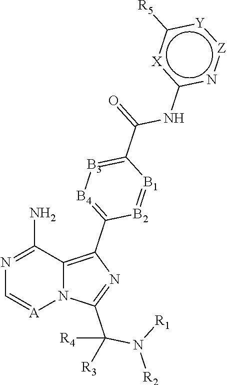

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of:

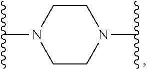 [SMILES: *N1CCN(*)CC1]

—O—, —(C₁₋₅)alkoxy-, and —[(C₁₋₁₀)alkyl]amino-;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

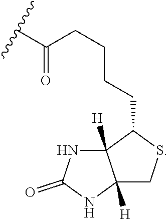 [SMILES: *C(=O)CCCC[C@@H]1SC[C@@H]2NC(=O)N[C@@H]21]

In an embodiment, the invention provides a method for determining a drug target occupancy of Bruton's tyrosine kinase (BTK) in a patient after treatment of the patient with a BTK inhibitor, comprising the steps of:

(a) obtaining a tissue sample from the patient;

(b) separating a population of cells from the tissue sample;

(c) contacting a BTK probe with the population of cells;

(d) detecting the amount of BTK bound to the BTK probe using an assay; and

(e) determining the drug target occupancy of BTK in the population of cells based on the amount of BTK bound to the BTK probe;

wherein the BTK probe is a compound according to:

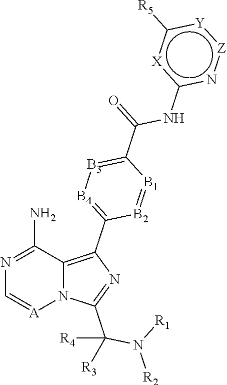

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is:

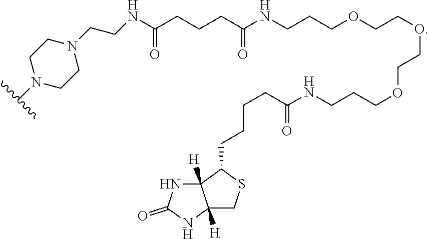 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

In an embodiment, the assay in any of the foregoing embodiments is an enzyme-linked immunosorbent assay (ELISA).

In an embodiment, the tissue sample in any of the foregoing embodiments is selected from the group consisting of blood, lymphatic tissue, and tumor biopsy tissue.

In an embodiment, the tissue sample in any of the foregoing embodiments is blood (including serum and plasma), and the population of cells are peripheral blood mononuclear cells.

In an embodiment, the BTK probe in any of the foregoing embodiments is selected from the group consisting of:

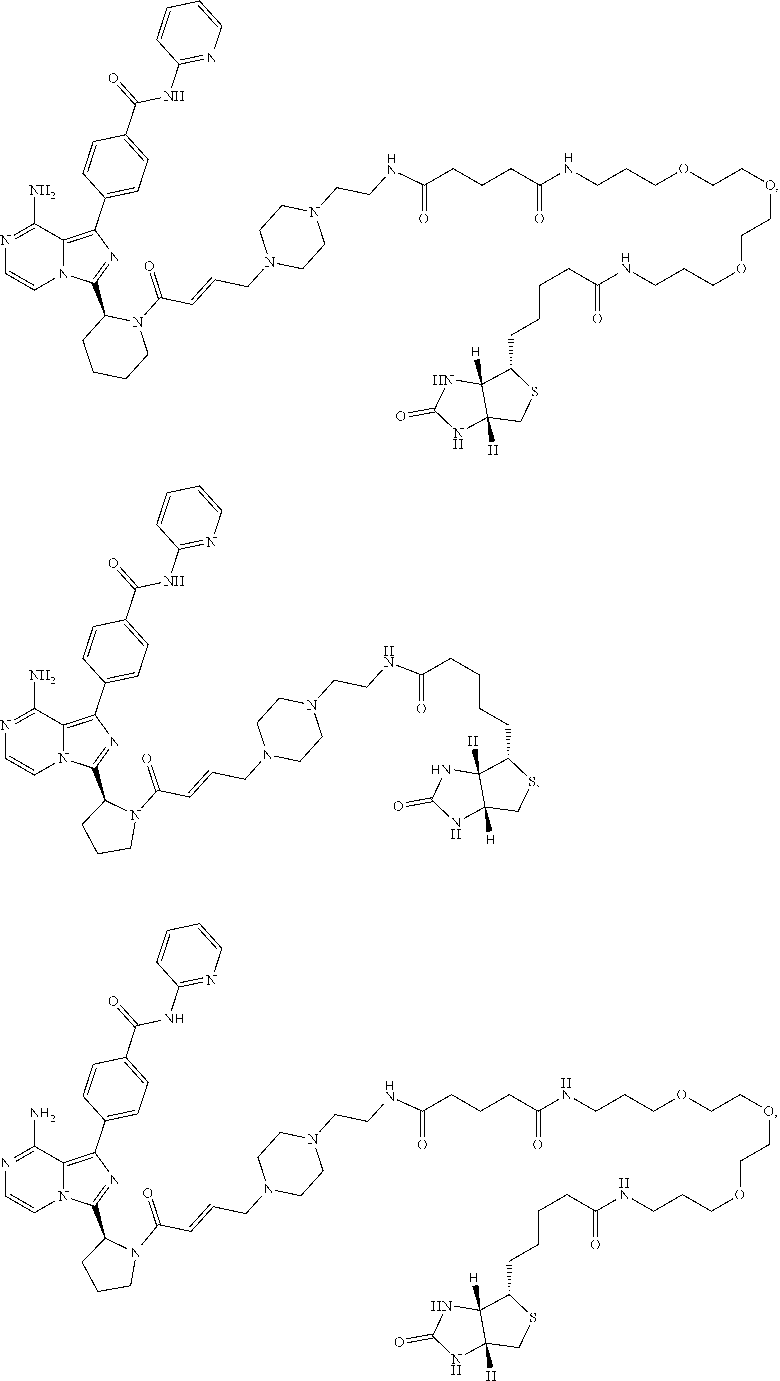 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12]

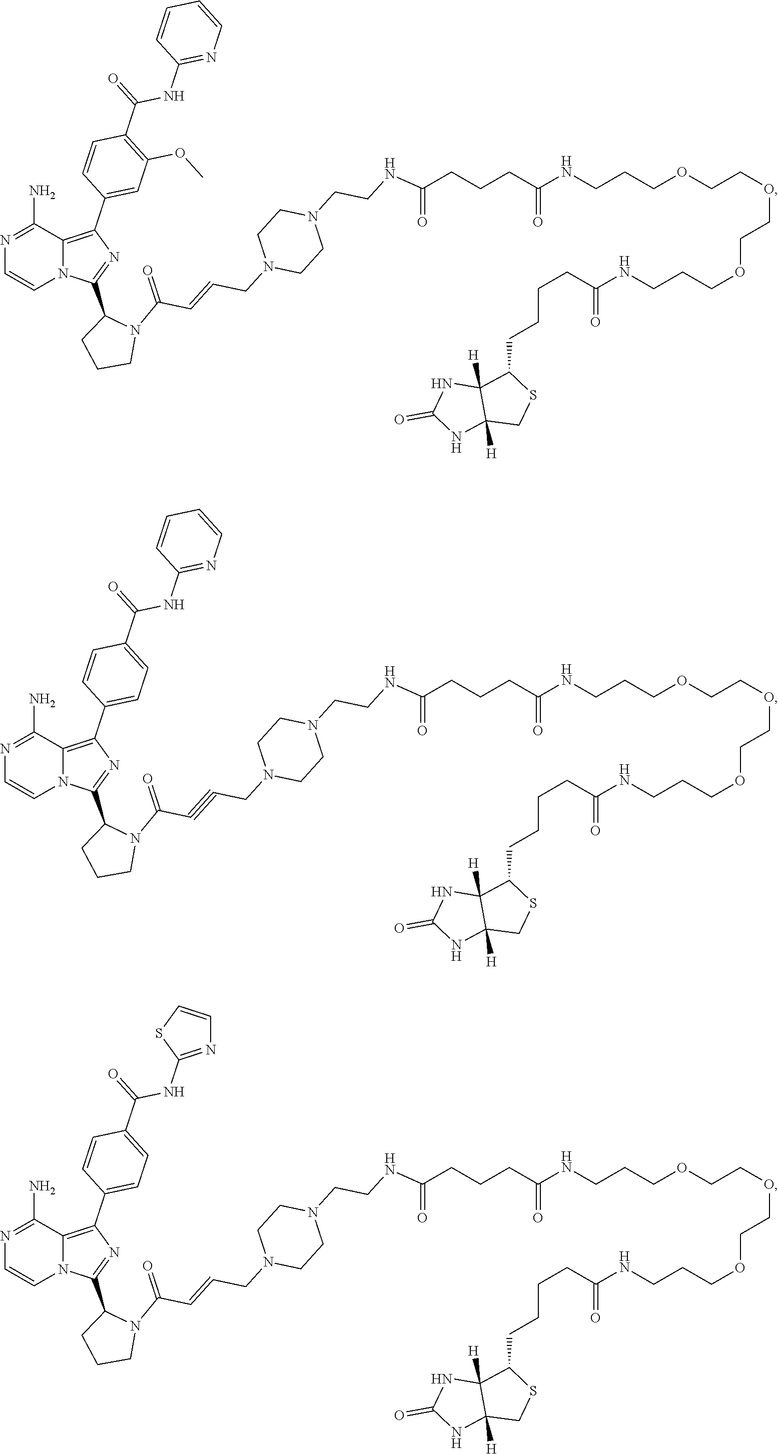 [SMILES: COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1 ; Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]

and salts or complexes thereof.

In an embodiment, the BTK inhibitor in any of the foregoing embodiments is selected from the group consisting of ibrutinib, acalabrutinib, ONO-4059, and pharmaceutically-acceptable salts, esters, prodrugs, cocrystals, solvates, or hydrates thereof.

In an embodiment, the BTK inhibitor in any of the foregoing embodiments is acalabrutinib.

In an embodiment, the methods of in any of the foregoing embodiments further comprise the step of adjusting a therapeutic regimen based on the drug target occupancy of BTK.

In an embodiment, the patient of in any of the foregoing embodiments is suffering from a BTK-mediated disorder. In an embodiment, the BTK-mediated disorder is selected from the group consisting of chronic lymphocytic leukemia, small lymphocytic leukemia, non-Hodgkin's lymphoma, diffuse large B cell lymphoma, follicular lymphoma, mantle cell lymphoma, Hodgkin's lymphoma, B cell acute lymphoblastic leukemia, Burkitt's lymphoma, Waldenström's macroglobulinemia, multiple myeloma, myelofibrosis, bladder cancer, head and neck cancer, pancreatic cancer, colon cancer, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, head and neck cancer, renal cancer, kidney cancer, liver cancer, prostate cancer, esophageal cancer, testicular cancer, gynecological cancer, thyroid cancer, glioblastoma, esophogeal tumors, hematological neoplasms, acquired immune deficiency syndrome (AIDS)-related lymphoma, Kaposi's sarcoma, viral-induced cancer, non-small-cell lung cancer, small-cell lung cancer, chronic myelocytic leukemia, hepatitis C virus infection, hepatocellular carcinoma, metastatic colon cancer, primary central nervous system lymphoma, ovary tumor, tumor angiogenesis, chronic inflammatory disease, rheumatoid arthritis, atherosclerosis, inflammatory bowel disease, psoriasis, eczema, scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcets disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, Crohn's Disease, lupus, and lupus nephritis.

In an embodiment, the invention provides a compound according to:

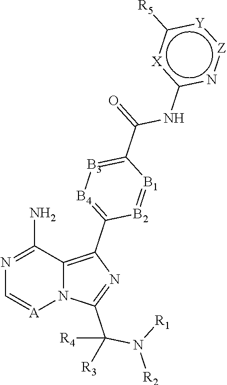

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of heterocycloalkyl and heteroalkyl;
   L₂ is a linear linker group selected from the group consisting of (C₁₋₅)alkylamide, (C₁₋₅)alkoxy, and a bond;
   L₃ is a linear linker group selected from the group consisting of (C₁₋₅)alkylamide, (C₁₋₅)alkoxy, and a bond;
   L₄ is a linear linker group selected from the group consisting of (C₁₋₅)alkylamide, (C₁₋₅)alkoxy, and a bond;
   m is 0 to 5;
   n is 0 to 5; and
   W is:

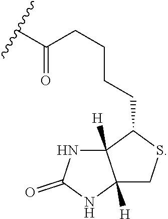 [SMILES: *C(=O)CCCC[C@@H]1SC[C@@H]2NC(=O)N[C@@H]21]

In an embodiment, the invention provides a compound according to:

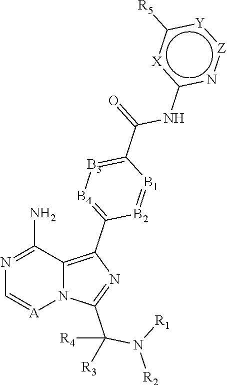

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of:

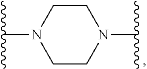 [SMILES: *N1CCN(*)CC1]

—O—, —(C₁₋₅)alkoxy-, and —[(C₁₋₁₀)alkyl]amino-;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

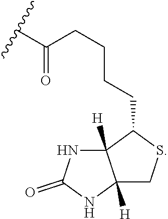 [SMILES: *C(=O)CCCC[C@@H]1SC[C@@H]2NC(=O)N[C@@H]21]

In an embodiment, the invention provides a compound according to:

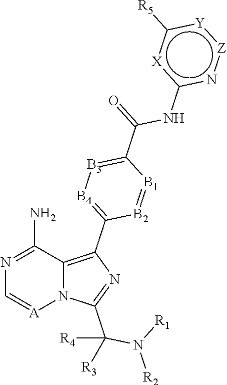

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is:

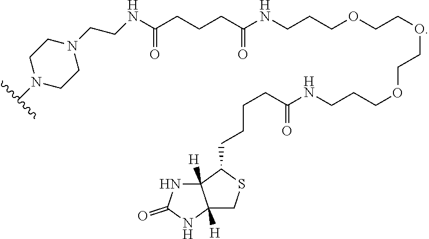 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

In an embodiment, the invention provides a compound selected from the group consisting of:

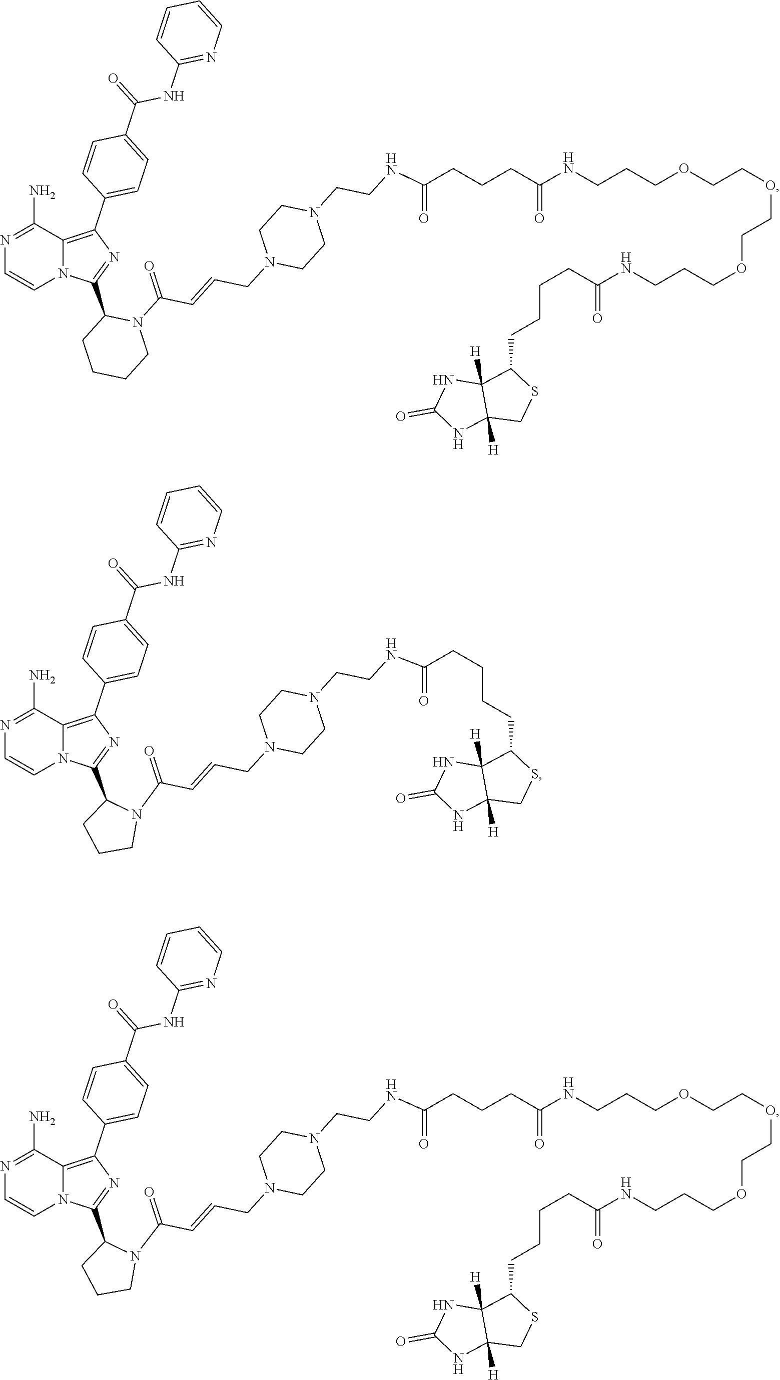 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12]

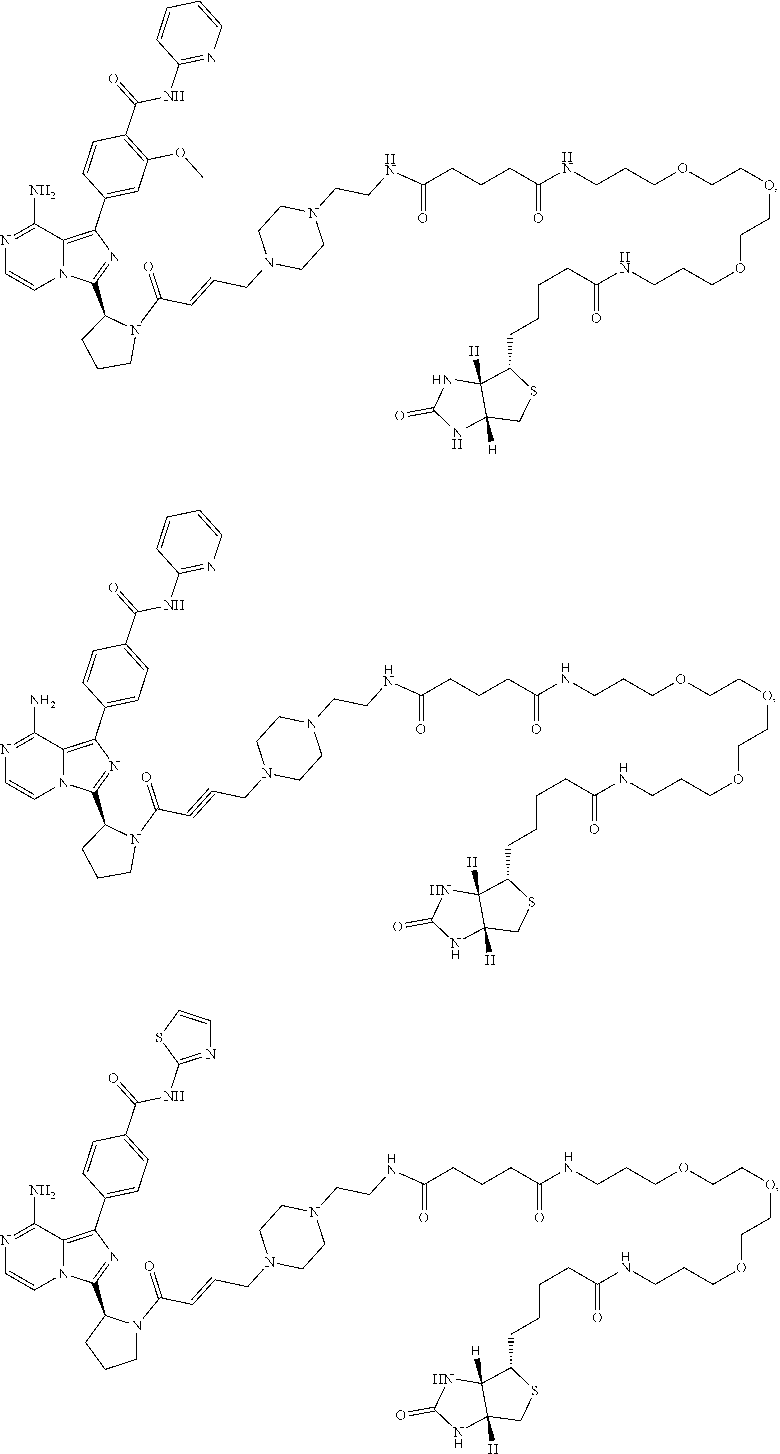 [SMILES: COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1 ; Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]

and salts or complexes thereof.

In an embodiment, the invention provides a kit comprising any of the foregoing compounds as a BTK probe. In an embodiment, the kit further comprises an enzyme-linked immunosorbent assay (ELISA).

### BRIEF DESCRIPTION OF THE DRAWINGS

The foregoing summary, as well as the following detailed description of the invention, will be better understood when read in conjunction with the appended drawings.

FIG. 1 illustrates the details for the different BTK target occupancy probes tested for the establishment of a BTK target occupancy assay.

FIG. 2 illustrates binding of BODIPY probes using different lysis buffers. Recombinant BTK was incubated with BTK target occupancy probes in different lysis buffers and after SDS-PAGE gel electrophoresis was measured for the fluorescence signal. Buffers used are PBS, lysis buffer 1 (50 mM Tris-HCl pH 7.5, 250 mM Sucrose, 5 mM MgCl₂, 1 mM DTT, 0.025% digitonin) and lysis buffer 2 (50 mM Tris-HCl pH 7.4, 150 mM NaCl, 1 mM EDTA, 1% Triton X100). BTK was incubated with the four different probes, denoted as 1 (Formula (5)), 2 (Formula (6)), 3 (Formula (3)), and 4 (Formula (4)).

FIG. 3 illustrates binding of biotin probes using different lysis buffers. Recombinant BTK was incubated with BTK target occupancy probes in different lysis buffers, run on a SDS-PAGE gel and transferred to PVDF membrane for Western blotting. The blot was probed with Streptavadin-horseradish peroxidase (HRP) for the detection of the biotin tagged probes bound to BTK. Buffers used are PBS, lysis buffer 1 (50 mM Tris-HCl pH 7.5, 250 mM Sucrose, 5 mM MgCl₂, 1 mM DTT, 0.025% digitonin) and lysis buffer 2 (50 mM Tris-HCl pH 7.4, 150 mM NaCl, 1 mM EDTA, 1% Triton X100). BTK was incubated with the four different probes, denoted as 1 (Formula (5)), 2 (Formula (6)), 3 (Formula (3)), and 4 (Formula (4)).

FIG. 4A, FIG. 4B, and FIG. 4C illustrate BTK target occupancy and target engagement in Ramos B cells treated with acalabrutinib. Ramos B cells are incubated for 2 hours in the presence or absence of a concentration range of acalabrutinib. Afterwards, cell pellets are lysed and used in the BTK target occupancy ELISA. Effects are shown in a bar graph (FIG. 4A) and a dose response curve using curve fitting (FIG. 4B). In FIG. 4A, the “0/+acalabrutinib” value indicates Ramos B cells not treated with acalabrutinib but where the cell lysate is incubated with exogenous acalabrutinib (1 μM). The value denoted with LB is obtained with lysis buffer only, without Ramos cell lysate. For the PLCγ2 phosphorylation, Ramos B cells were incubated for 2 hours with a concentration range of acalabrutinib, followed by a 10 minute stimulation with 100 mM H₂O₂. Cell lysates were run on SDS-PAGE gel and Western blotted. The blot is probed with anti-pPLCγ2. In FIG. 4C, the actual result of the Western blot together with the dose response curve based on the quantification of the signal observed on the blot is shown.

FIG. 5 illustrates BTK target occupancy for canine peripheral B cells. Cell lysates of CD21+ cells from a dog prior to dosing with acalabrutinib (predose), 3 hours after dosing, and on day 7 prior to repeat dosing were used in the BTK target occupancy ELISA. In addition, predose cell lysates of CD21− cells were profiled. Cell lysates were incubated in the presence or absence of exogenous acalabrutinib (1 μM) prior to incubation with the BTK probe of Formula (3).

FIG. 6 illustrates human PBMC cell numbers for BTK target occupancy by ELISA. Cell lysates from the indicated number of human PBMCs were incubated in the presence or absence of exogenous acalabrutinib (1 μM) prior to incubation with the BTK probe of Formula (3). Analysis of free BTK signal was performed using the BTK target occupancy ELISA procedure.

FIG. 7A and FIG. 7B illustrates the dose response with acalabrutinib in human PBMCs on BTK target occupancy and target engagement. Human PBMCs are incubated for 2 hours in the presence or absence of a concentration range of acalabrutinib. Following this incubation, either cell lysates were prepared for target occupancy (FIG. 7A) or PBMCs were stimulated for 10 minutes with anti-IgM [10 μg/mL]+H₂O₂[3.3 mM] for PLCγ2 phosphorylation in gated CD20+ B cells (FIG. 7B).

FIG. 8A, FIG. 8B, FIG. 8C, and FIG. 8D illustrate the BTK occupancy when Lysate from Ramos cells treated with 100 nM acalabrutinib (fully occupied BTK) or DMSO control (unoccupied BTK) was mixed at different ratios to model expected assay occupancy. Expected occupancy is represented on the x-axis, while measured occupancy is shown on the y-axis. Each point represents a single Ramos dilution, with error bars representing SD of replicate values. Dotted lines represent the expected calibration curve. Data for 400K Ramos are shown in FIG. 8A, with the occupancy values and % of expected shown in FIG. 8 B. Data using 40K Ramos, generated from the same lysates diluted 1:10 before mixing, are shown in FIG. 8 C, with the occupancy and % of expected shown in FIG. 8D.

FIG. 9A, FIG. 9B, and FIG. 9C illustrate the BTK occupancy when Ramos cells were treated with varying doses of acalabrutinib, made into pellets, and stored at −80° C. BTK TO assay was performed on three separate days, with three replicates per plate. Corrected signal (signal—background) for each dose is shown in FIG. 9A. Percent occupied BTK was calculated by normalizing against signal from untreated Ramos cells FIG. 9B. A summary of the data is shown in FIG. 9C.

FIG. 10A, FIG. 10B, FIG. 10C, FIG. 10D, and FIG. 10E, illustrate dilution linearity of BTK target occupancy by ELISA. Serial dilutions of Ramos lysate were performed to test signal linearity. Corrected luminescence signal, calculated by subtracting background from signal, is shown in FIG. 10A, with linear regression for signal values representing 1.25×105 or more cells. The lower end of the signal, from 7.8×103 to 1.25×105 cells, is magnified in FIG. 10B, with linear regression encompassing those values. Each point represents a single Ramos concentration, with error bars representing SD of replicate values. Signal-to-background (S/N) ratio at each dilution from two independent runs is shown in FIG. 10C, with the lower end magnified in FIG. 10D. Data used to create plots FIGS. 10A-D are shown in FIG. 10E

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs. All patents and publications referred to herein are incorporated by reference in their entireties.

### Definitions

The term “BTK inhibitor” refers to any molecule capable of inhibiting BTK. BTK inhibitors may inhibit BTK through mechanisms that include both covalent and non-covalent binding. For example, ibrutinib, ONO-4059, acalabrutinib, and CC-292 are BTK inhibitors, with the following chemical structures:

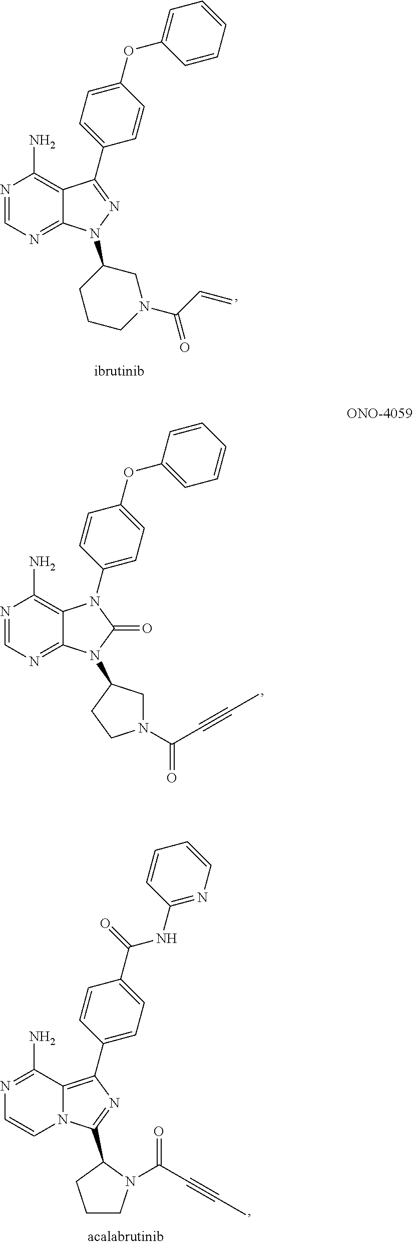 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; CC#CC(=O)N1CCC[C@H]1c1nc(-c2ccc(C(=O)Nc3ccccn3)cc2)c2c(N)nccn12]

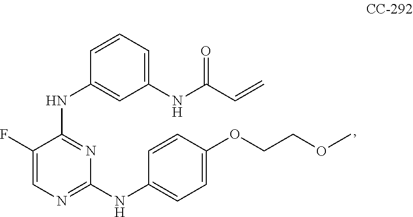 [SMILES: C=CC(=O)Nc1cccc(Nc2nc(Nc3ccc(OCCOC)cc3)ncc2F)c1]

and pharmaceutically-acceptable salts, cocrystals, hydrates, solvates, and prodrugs thereof. BTK inhibitors also include compounds according to the following chemical structures:

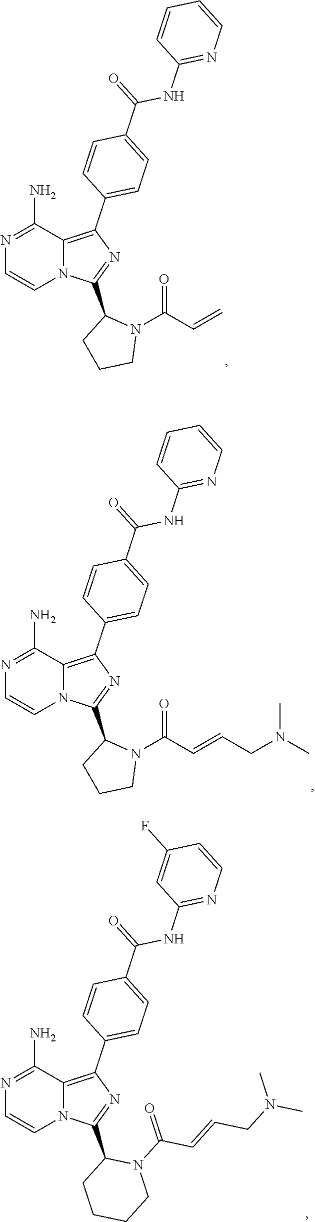

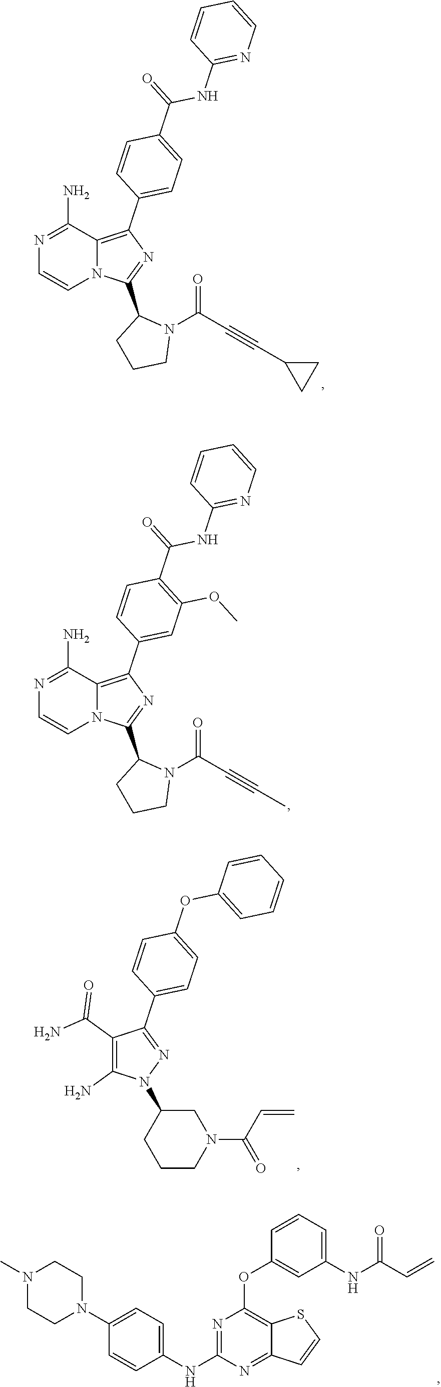 [SMILES: Nc1nccn2c([C@@H]3CCCN3C(=O)C#CC3CC3)nc(-c3ccc(C(=O)Nc4ccccn4)cc3)c12 ; CC#CC(=O)N1CCC[C@H]1c1nc(-c2ccc(C(=O)Nc3ccccn3)c(OC)c2)c2c(N)nccn12 ; C=CC(=O)N1CCC[C@@H](n2nc(-c3ccc(Oc4ccccc4)cc3)c(C(N)=O)c2N)C1 ; C=CC(=O)Nc1cccc(Oc2nc(Nc3ccc(N4CCN(C)CC4)cc3)nc3ccsc23)c1]

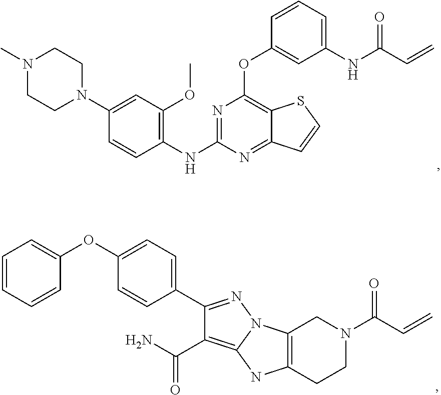 [SMILES: C=CC(=O)Nc1cccc(Oc2nc(Nc3ccc(N4CCN(C)CC4)cc3OC)nc3ccsc23)c1 ; C=CC(=O)N1CCc2[nH]c3c(C(N)=O)c(-c4ccc(Oc5ccccc5)cc4)nn3c2C1]

and pharmaceutically-acceptable salts, cocrystals, hydrates, solvates, and prodrugs thereof. BTK inhibitors include compounds described in International Patent Application Publication No. WO 2013/010868 and U.S. Patent Application Publication No. US 2014/0155385 A1; International Patent Application Publication No. WO 2013/010869 and U.S. Patent Application Publication No. US 2014/0155406 A1; U.S. Pat. Nos. 8,957,065; 8,450,335 and 8,609,679 and U.S. Patent Application Publication Nos. US 2010/0029610 A1, US 2012/0077832 A1, US 2013/0065879 A1, US 2013/0072469 A1, and US 2013/0165462 A1; and International Patent Application Publication No. WO 2013/081016 A1 and U.S. Patent Application Publication No. US 2014/0330015 A1, the disclosures of each of which are incorporated herein by reference.

The term “BTK probe,” as used herein, refers to molecules capable of assessing BTK target occupancy.

The terms “co-administration,” “co-administering,” “administered in combination with,” and “administering in combination with” as used herein, encompass administration of two or more agents to a subject so that both agents and/or their metabolites are present in the subject at the same time. Co-administration includes simultaneous administration in separate compositions, administration at different times in separate compositions, or administration in a composition in which two or more agents are present.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the described compositions.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, Design of Prodrugs, Elsevier, Amsterdam, 1985). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylamide” refers to an -(alkyl)amide radical, where aryl and alkyl are as disclosed herein and which are optionally substituted by one or more of the substituents described as suitable substituents for aryl and alkyl respectively. Alkylamide includes all arrangements of the amide group, including —C(O)NH-alkyl-, -alkyl-C(O)NH—, —NHC(O)-alkyl-, -alkyl-NHC(O)—, -alkyl-NHC(O)-alkyl-, and -alkyl-C(O)NH-alkyl.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein “R” is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” is intended to mean fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl”, “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolin yl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 3-oxopiperazinyl, 2-oxomorpholinyl, 3-oxomorpholinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 2-oxothiomorpholinyl, 3-oxothiomorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York, 1981; Eliel, Stereochemistry of Carbon Compounds, McGraw-Hill, N Y, 1962; and Eliel and Wilen, Stereochemistry of Organic Compounds, Wiley, New York, 1994.

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed after the selective reaction is complete. A variety of protecting groups are disclosed, for example, in T. H. Greene and P. G. M. Wuts, Protective Groups in Organic Synthesis, Third Edition, John Wiley & Sons, New York, 1999.

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Wavy lines ( [symbol] ) signify an attachment point for a functional group, including the foregoing functional groups.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### BTK Probes

BTK probes of the present invention include BTK probes that bind covalently to the target (in an irreversible manner) and BTK probes that bind non-covalently to the target (in a reversible manner). In an embodiment, the BTK probe binds covalently to the cysteine residue at position 481 of BTK.

In an embodiment, the BTK probe is a compound according to Formula (1):

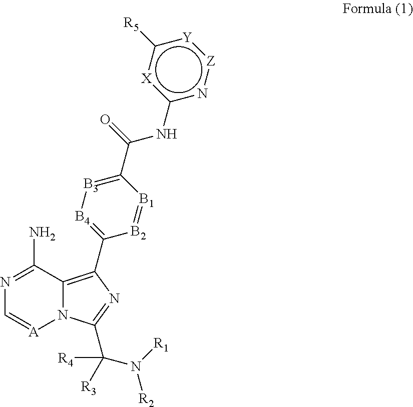

or a salt or complex thereof, wherein:
X is CH or S;
Y is C(R₆);
Z is CH or bond;
A is CH or N;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of heterocycloalkyl and heteroalkyl;
   L₂ is a linear linker group selected from the group consisting of (C₁₋₅)alkylamide, (C₁₋₅)alkoxy, and a bond;
   L₃ is a linear linker group selected from the group consisting of (C₁₋₅)alkylamide, (C₁₋₅)alkoxy, and a bond;
   L₄ is a linear linker group selected from the group consisting of (C₁₋₅)alkylamide, (C₁₋₅)alkoxy, and a bond;
   m is 0 to 5;
   n is 0 to 5; and
   W is:

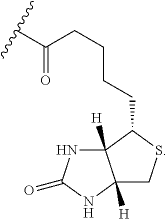 [SMILES: *C(=O)CCCC[C@@H]1SC[C@@H]2NC(=O)N[C@@H]21]

In an embodiment, the BTK probe is a compound according to Formula (1) or a salt or complex thereof, wherein:
X is CH or S;
Y is C(R₆);
Z is CH or bond;
A is CH or N;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂₋(L₃)ₘ-(L₄-)ₙ-W, wherein:
   L₁ is selected from the group consisting of consisting of

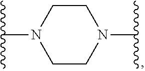 [SMILES: *N1CCN(*)CC1]

—O—, —(C₁₋₅)alkoxy-, and —[(C₁₋₁₀)alkyl]amino-;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

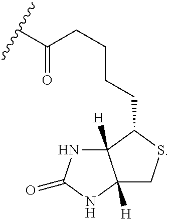 [SMILES: *C(=O)CCCC[C@@H]1SC[C@@H]2NC(=O)N[C@@H]21]

In an embodiment, the BTK probe is a compound according to Formula (1) or a salt or complex thereof, wherein:
X is CH or S;
Y is C(R₆);
Z is CH or bond;
A is CH or N;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is:

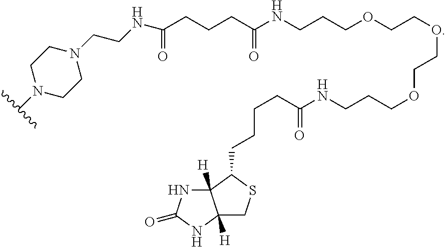 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

In an embodiment, the BTK probe is a compound according to Formula (2):

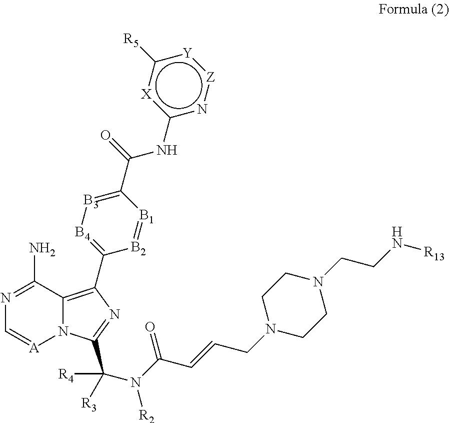

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N, and wherein R₁₃ is a tag group.

In an embodiment, the tag group is selected from the group consisting of a fluorophore, a chemiluminophore, and an electrochemiluminophore. In an embodiment, the tag group comprises a BODIPY (boron-dipyrromethene) tag. In an embodiment, the tag group comprises a biotin tag. In an embodiment, the tag group comprises a Texas Red sulfonyl chloride tag. In an embodiment, the tag group comprises a BODIPY-Texas Red tag. In an embodiment, the tag group comprises a 5-carboxyrhodamine 6G hydrochloride tag. In an embodiment, the tag group comprises a lissamine rhodamine B sulfonyl chloride tag. In an embodiment, the tag group comprises a carboxytetramethylrhodamine (TAMRA) tag. In an embodiment, the tag group comprises a 7-nitrobenz-2-oxa-1,3-diazole (NBD) tag. In an embodiment, the tag group comprises tris(bipyridine)ruthenium(II) dichloride. In an embodiment, the tag group comprises ruthenium (II) tris-bipyridine, N-hydroxysuccinimide.

In an embodiment, the tag group is selected from the group consisting of chemical labels, biochemical labels, biological labels, colorimetric labels, enzymatic labels, fluorescent labels, luminescent labels, chemiluminescent labels, and electrochemiluminescent labels. In an embodiment, the tag group is selected from the group consisting of a dye, a photocrosslinker, a cytotoxic compound, a drug, an affinity label, a photoaffinity label, a reactive compound, an antibody or antibody fragment, a biomaterial, a nanoparticle, a quantum dot, a spin label, a fluorophore, a metal-containing moiety, a radioactive moiety, a group that covalently or noncovalently interacts with other molecules, a photocaged moiety, an actinic radiation excitable moiety, a ligand, a photoisomerizable moiety, biotin, a biotin analogue, a moiety incorporating a heavy atom, a chemically cleavable group, a photocleavable group, a redox-active agent, an isotopically labeled moiety, a biophysical probe, a phosphorescent group, a chemiluminescent group, a magnetic group, an intercalating group, a chromophore, an energy transfer agent, a biologically active agent, a detectable label, or a combination thereof.

In an embodiment, the BTK probe is a compound of Formula (2), wherein R₁₃ is selected from the group consisting of:

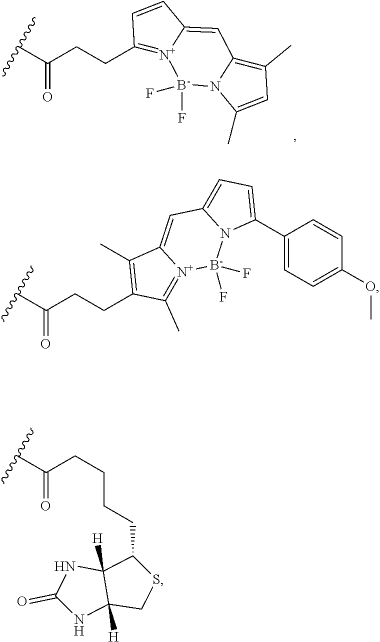

and pharmaceutically acceptable salts, solvates, hydrates, and cocrystals thereof.

In an embodiment, the BTK probe is a compound selected from the group consisting of:

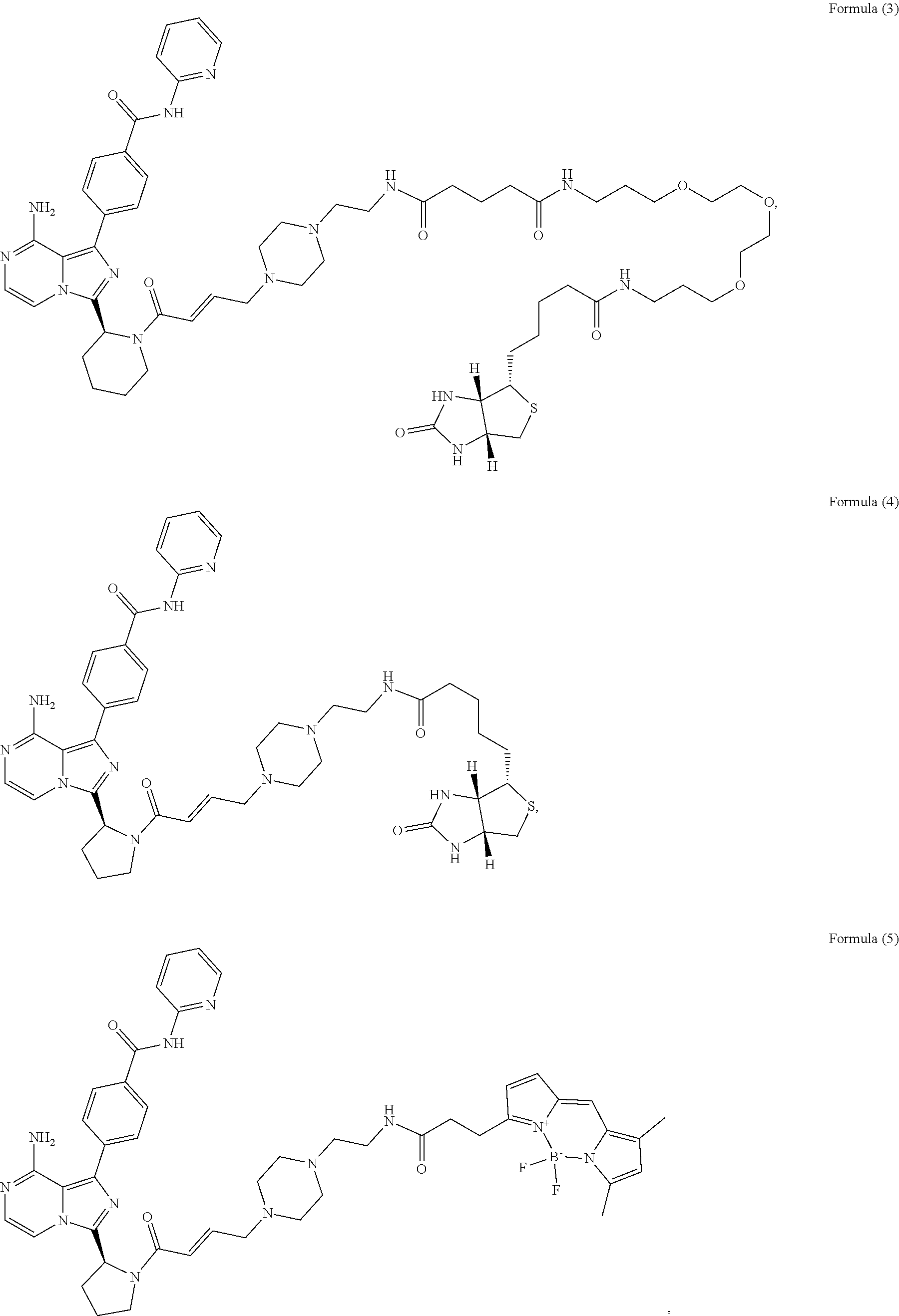 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12 ; Cc1cc(C)n2c1C=C1C=CC(CCC(=O)NCCN3CCN(C/C=C/C(=O)N4CCC[C@H]4c4nc(-c5ccc(C(=O)Nc6ccccn6)cc5)c5c(N)nccn45)CC3)=[N+]1[B-]2(F)F]

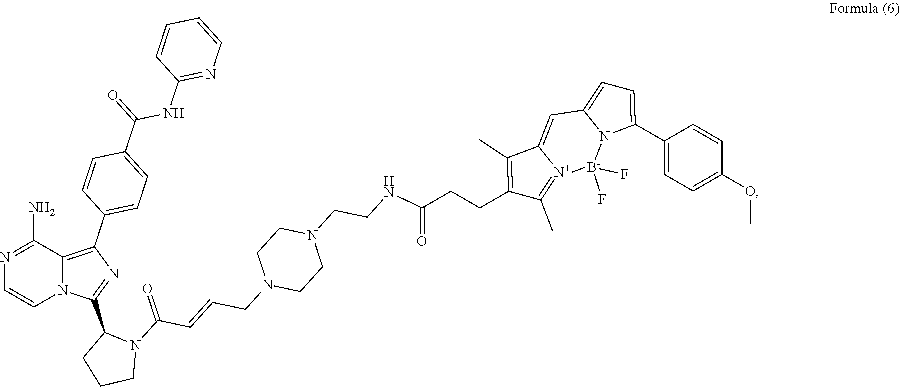 [SMILES: COc1ccc(-c2ccc3n2[B-](F)(F)[N+]2=C(C)C(CCC(=O)NCCN4CCN(C/C=C/C(=O)N5CCC[C@H]5c5nc(-c6ccc(C(=O)Nc7ccccn7)cc6)c6c(N)nccn56)CC4)=C(C)C2=C3)cc1]

and pharmaceutically acceptable salts, solvates, hydrates, and cocrystals thereof.

In an embodiment, the BTK probe is a compound selected from the group consisting of:

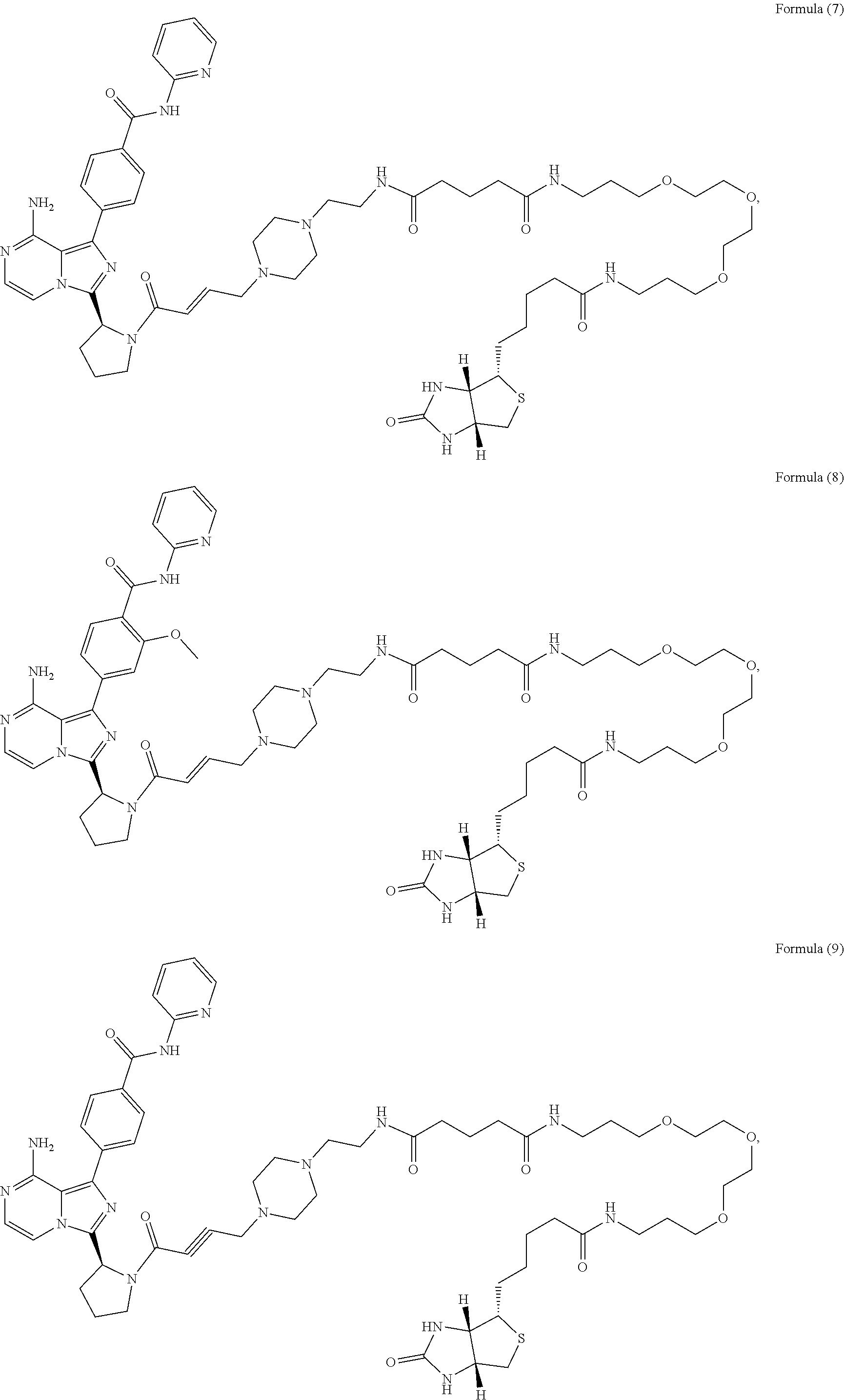 [SMILES: Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1 ; Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12]

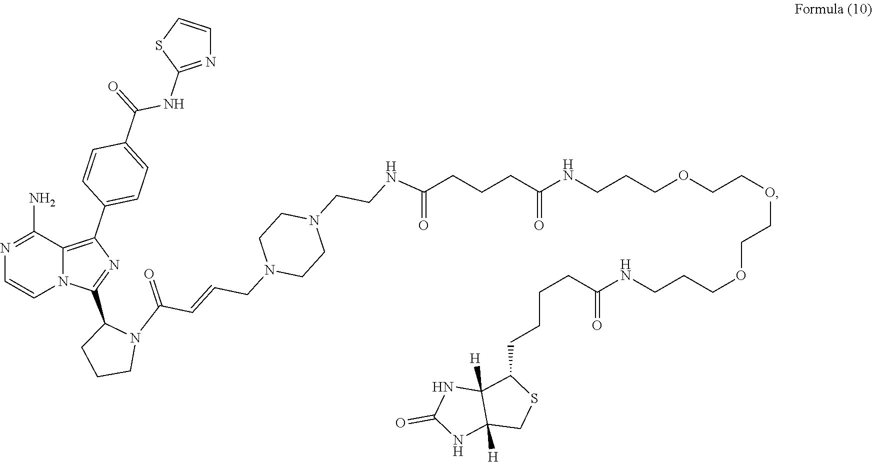 [SMILES: Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]

and pharmaceutically acceptable salts, solvates, hydrates, and cocrystals thereof.

In an embodiment, the invention provides a compound according to:

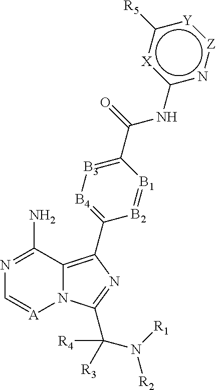

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of heterocycloalkyl and heteroalkyl;
   L₂ is a linear linker group selected from the group consisting of (C₁₋₅)alkylamide, (C₁₋₅)alkoxy, and a bond;
   L₃ is a linear linker group selected from the group consisting of (C₁₋₅)alkylamide, (C₁₋₅)alkoxy, and a bond;
   L₄ is a linear linker group selected from the group consisting of (C₁₋₅)alkylamide, (C₁₋₅)alkoxy, and a bond;
   m is 0 to 5;
   n is 0 to 5; and
   W is:

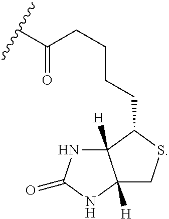 [SMILES: *C(=O)CCCC[C@@H]1SC[C@@H]2NC(=O)N[C@@H]21]

In an embodiment, the invention provides a compound according to:

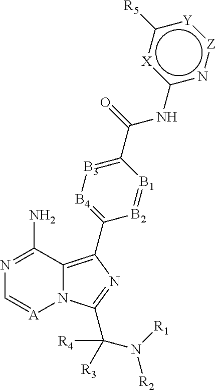

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
   L₁ is selected from the group consisting of:

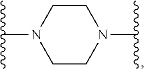 [SMILES: *N1CCN(*)CC1]

—O—, —(C₁₋₅)alkoxy-, and —[(C₁₋₁₀)alkyl]amino-;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

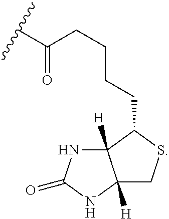 [SMILES: *C(=O)CCCC[C@@H]1SC[C@@H]2NC(=O)N[C@@H]21]

In an embodiment, the invention provides a compound according to:

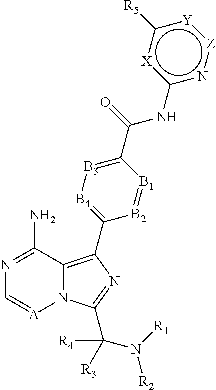

or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is:

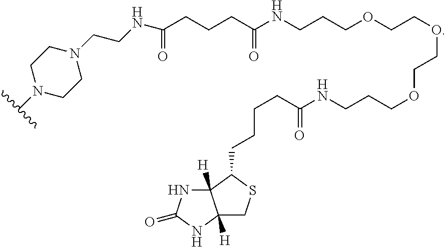 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

In an embodiment, the invention provides a compound selected from the group consisting of:

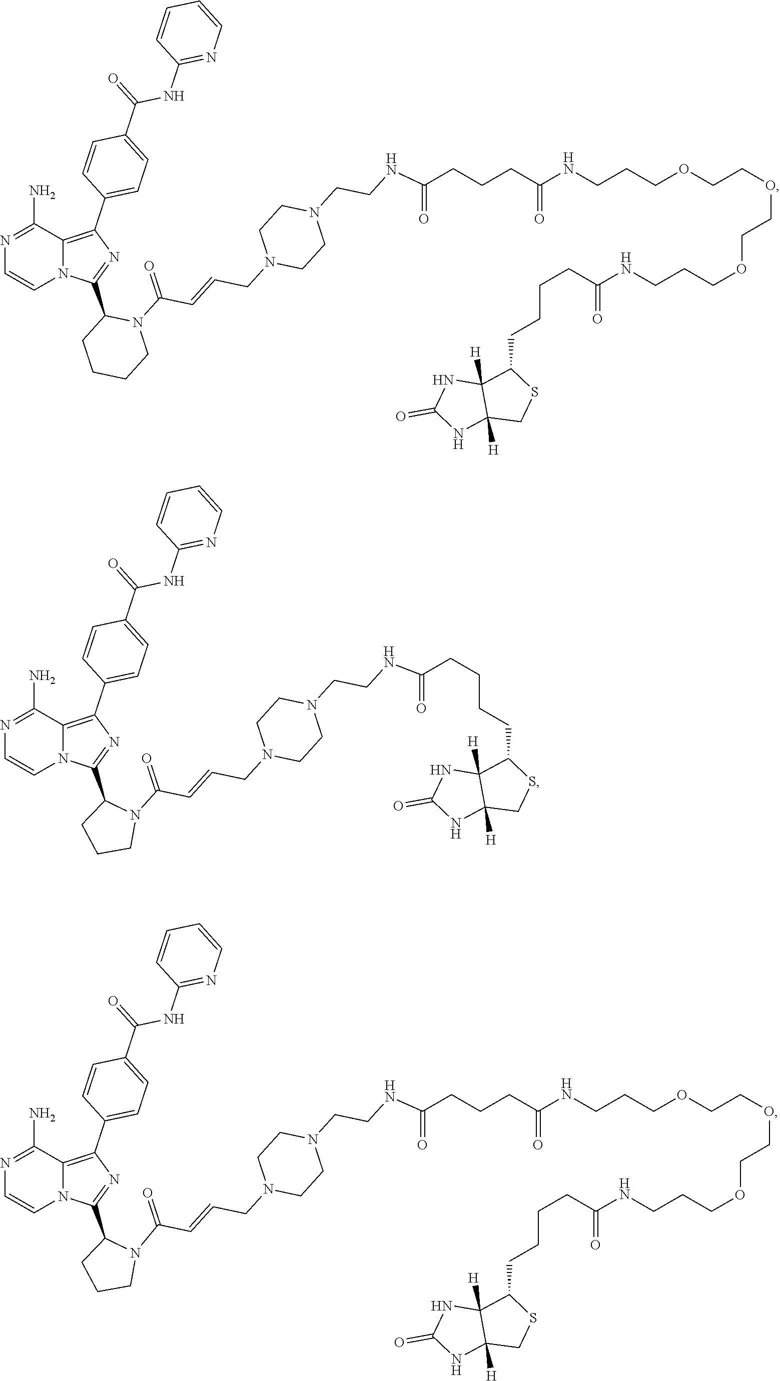 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12]

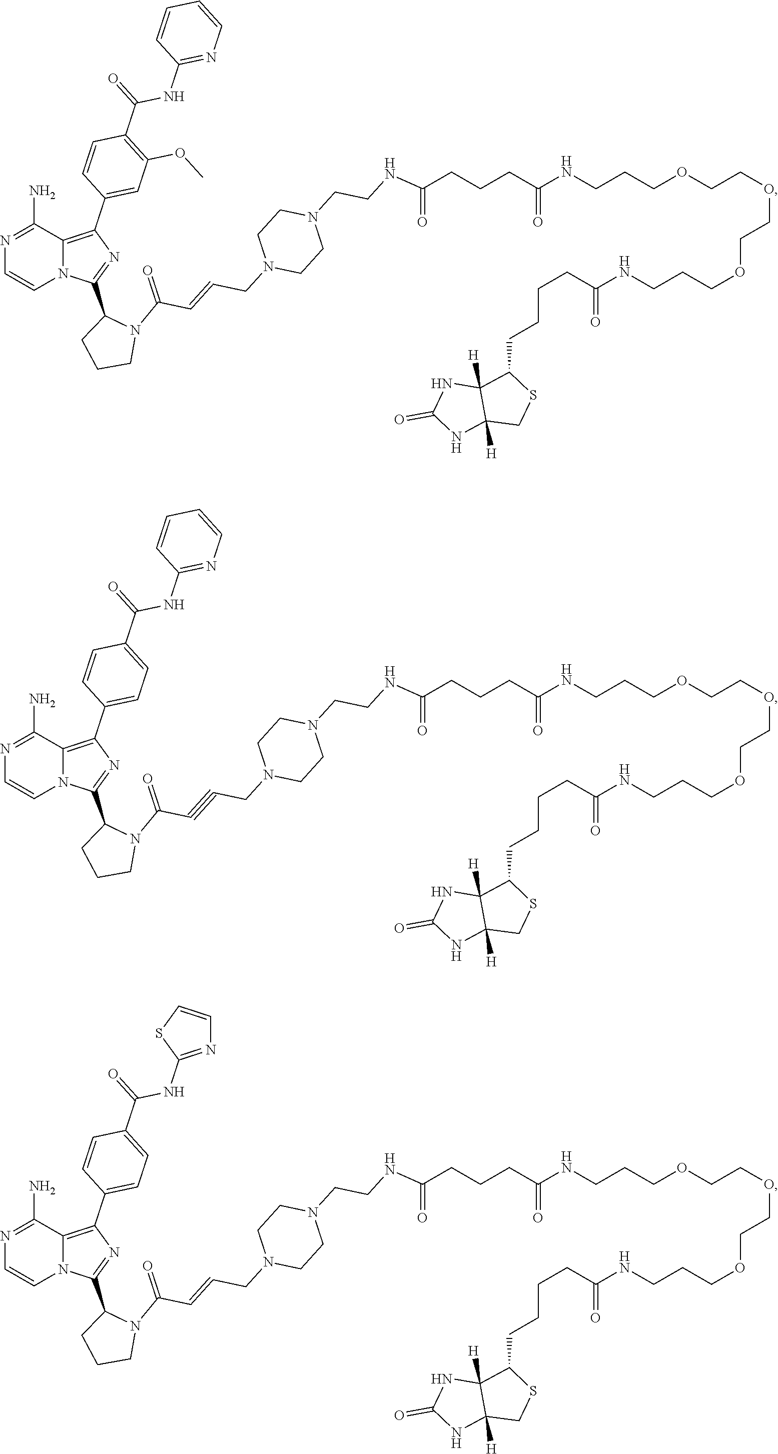 [SMILES: COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1 ; Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]

and salts or complexes thereof.

In an embodiment, the invention provides a kit comprising any of the foregoing compounds as a BTK probe. In an embodiment, the kit further comprises an enzyme-linked immunosorbent assay (ELISA). In an embodiment, the kit further comprises an assay for PLCγ2 phosphorylation.

In some embodiments, the concentration of each of the BTK probes provided in the kits or compositions of the invention is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v, relative to the total mass or volume of the pharmaceutical composition.

In some embodiments, the concentration of each of the BTK probes provided in the kits or compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v, relative to the total mass or volume of the pharmaceutical composition.

In some embodiments, the concentration of each of the BTK probes of the kits and compositions of the invention is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v, relative to the total mass or volume of the pharmaceutical composition.

In some embodiments, the concentration of each of the BTK probes of the kits and compositions of the invention is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v, relative to the total mass or volume of the pharmaceutical composition.

In some embodiments, the amount of each of the BTK probes in the kits and compositions of the invention is independently equal to or less than 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In some embodiments, the amount of each of the BTK probes in the kits and compositions of the invention is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, or 3 g.

### BTK-Mediated Diseases Affecting Patients Undergoing Assessment for BTK Drug Target Occupancy

In some embodiments, the invention relates to methods for determining a drug target occupancy of Bruton's tyrosine kinase (BTK) in a patient after treatment of the patient with a BTK inhibitor, comprising the steps of: (a) obtaining a tissue sample from the patient; (b) separating a population of cells from the tissue sample; (c) contacting a BTK probe with the population of cells; (d) detecting the amount of BTK bound to the BTK probe using an assay; and (e) determining the drug target occupancy of BTK in the population of cells based on the amount of BTK bound to the BTK probe, wherein the patient is suffering from a BTK-mediated disease. In some embodiments, the patient is suffering from a BTK-mediated disease selected from the group consisting of a hyperproliferative disorder, an inflammatory disorder, an immune disorder, and an autoimmune disorder in a mammal.

In some embodiments, the patient is suffering from a hyperproliferative disorder selected from the group consisting of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, head, neck, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, primary central nervous system lymphoma, non-small-cell lung cancer (NSCLC), chronic myelocytic leukemia, diffuse large B-cell lymphoma (DLBCL), esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, ovary tumor, pancreas tumor, renal cell carcinoma, small-cell lung cancer, stage IV melanoma and marginal zone lymphoma (MZL).

In some embodiments, the patient is suffering from a hyperproliferative disorder, including but not limited to cancer such as acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS-related (e.g., lymphoma and Kaposi's sarcoma) or viral-induced cancer.

In some embodiments, the patient is suffering from a hyperproliferative disorder that is a solid tumor cancer selected from the group consisting of bladder cancer, squamous cell carcinoma, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancers such as cervical carcinoma (human papillomavirus), B-cell lymphoproliferative disease, nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), glioblastoma, esophogeal tumors, head and neck tumor, metastatic colon cancer, head and neck squamous cell carcinoma, ovary tumor, pancreas tumor, renal cell carcinoma, hematological neoplasms, small-cell lung cancer, non-small-cell lung cancer, stage IV melanoma, and glioma.

In some embodiments, the patient is suffering from a hyperproliferative disorder that is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, myelodysplatic syndromes, myelofibrosis, and marginal zone lymphoma (MZL). In some embodiments, the patient is suffering from a cancer, wherein the cancer is chronic myelocytic leukemia, acute myeloid leukemia, DLBCL (including activated B-cell (ABC) and germinal center B-cell (GCB) subtypes), follicle center lymphoma, Hodgkin's disease, multiple myeloma, indolent non-Hodgkin's lymphoma, marginal zone lymphoma (MZL), and mature B-cell ALL.

In some embodiments, the patient is suffering from a hyperproliferative disorder that is a subtype of CLL. A number of subtypes of CLL have been characterized. CLL is often classified for immunoglobulin heavy-chain variable-region (IgV_{H}) mutational status in leukemic cells. Damle, et al., Blood 1999, 94, 1840-47; Hamblin, et al., Blood 1999, 94, 1848-54. Patients with IgV_{H} mutations generally survive longer than patients without IgV_{H} mutations. ZAP70 expression (positive or negative) is also used to characterize CLL. Rassenti, et al., N. Engl. J. Med. 2004, 351, 893-901. The methylation of ZAP-70 at CpG3 is also used to characterize CLL, for example by pyrosequencing. Claus, et al., J. Clin. Oncol. 2012, 30, 2483-91; Woyach, et al., Blood 2014, 123, 1810-17. CLL is also classified by stage of disease under the Binet or Rai criteria. Binet, et al., Cancer 1977, 40, 855-64; Rai and Han, Hematol. Oncol. Clin. North Am. 1990, 4, 447-56. Other common mutations, such as 11q deletion, 13q deletion, and 17p deletion can be assessed using well-known techniques such as fluorescence in situ hybridization (FISH). In an embodiment, the invention relates to a method of treating a CLL in a human, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In some embodiments, the patient is suffering from a hyperproliferative disorder, wherein the hyperproliferative disorder is CLL that has undergone a Richter's transformation. Methods of assessing Richter's transformation, which is also known as Richter's syndrome, are described in Jain and O'Brien, Oncology, 2012, 26, 1146-52. Richter's transformation is a subtype of CLL that is observed in 5-10% of patients. It involves the development of aggressive lymphoma from CLL and has a generally poor prognosis.

In some embodiments, the patient is suffering from a hyperproliferative disorder selected from the group consisting of CLL and SLL, wherein the patient is sensitive to lymphocytosis. In some embodiments, the patient is suffering from CLL or SLL, wherein the patient exhibits lymphocytosis caused by a disorder selected from the group consisting of a viral infection, a bacterial infection, a protozoal infection, or a post-splenectomy state. In an embodiment, the viral infection in any of the foregoing embodiments is selected from the group consisting of infectious mononucleosis, hepatitis, and cytomegalovirus. In an embodiment, the bacterial infection in any of the foregoing embodiments is selected from the group consisting of pertussis, tuberculosis, and brucellosis.

In some embodiments, the patient is suffering from a hyperproliferative disorder selected from the group consisting of myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis.

In some embodiments, the patient is suffering from a non-cancerous hyperproliferative disorder selected from the group consisting of benign hyperplasia of the skin, restenosis, and benign prostatic hypertrophy (BPH).

In some embodiments, the patient is suffering from an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's disease, lupus, and lupus nephritis.

In some embodiments, the patient is suffering from an inflammatory, immune, or autoimmune disorder that is a disease related to vasculogenesis or angiogenesis, including tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In some embodiments, the patient is suffering from an inflammatory, immune, or autoimmune disorder selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcets disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, Crohn's Disease, lupus, and lupus nephritis.

In some embodiments, the patient is suffering from a neurodegenerative disorder selected from the group consisting of Parkinson's disease, sporadic and familial Alzheimer's disease, neurodegenerative tauopathies, mild cognitive impairment, vascular dementia (VD), Down's syndrome, Lewy body variant of Alzheimer's disease, Guillain-Barré syndrome, chronic inflammatory demyelinating polyneuropathy, chronic encephalomyelitis, Pick's disease, corticobasal degeneration, progressive supranuclear palsy, frontotemporal dementia with Parkinsonism linked to chromosome 17 or FTDP-17, amyotrophic lateral sclerosis (ALS or Lou Gehrig's disease), sporadic or hereditary amyotrophic lateral sclerosis, polyglutamine or trinucleotide repeat diseases, Huntington's disease, sporadic and familial synucleinopathies, dementia with Lewy bodies, multiple system atrophy, neurodegeneration with brain iron accumulation, neuronal intranuclear inclusion disease, hereditary spastic paraplegias, Ménière's disease, chronic fatigue syndrome, Charcot-Marie-Tooth disease, and sporadic or hereditary prion disease, Gaucher disease, Tay Sachs disease, Farber's disease, Niemann-Pick disease (including Types A, B & C), GM1 gangliosidosis, GM2 gangliosidosis, mucopolysaccharidosis type I (including Hurler, Hurler-Scheie, and Scheie syndromes), multiple sclerosis, clinically isolated syndrome, relapsing-remitting multiple sclerosis, malignant multiple sclerosis, primary progressive multiple sclerosis, secondary progressive multiple sclerosis, neuromyelitis optica spectrum diseases, Devic's syndrome, Balo concentric sclerosis, Marburg multiple sclerosis, diffuse myelinoclastic sclerosis, chronic focal encephalitis, Rasmussen's encephalitis, acute disseminated encephalomyelitis, Lyme encephalopathy, stiff person syndrome, mild cognitive impairment, cerebral amyloid angiopathy, Lewy body disease, frontotemporal dementia (FTD), multiple system atrophy (MSA), progressive supranuclear palsy, movement disorders (including ataxia, cerebral palsy, choreoathetosis, dystonia, Tourette's syndrome, kernicterus), tremor disorders, leukodystrophies (including adrenoleukodystrophy, metachromatic leukodystrophy, Canavan disease, Alexander disease, Pelizaeus-Merzbacher disease), neuronal ceroid lipofucsinoses, ataxia telangectasia, Rett syndrome, Hallervorden-Spatz disease, progressive familial myoclonic epilepsy, striatonigral degeneration, progressive supranuclear palsy, torsion dystonia (torsion spasm; dystonia musculorum deformans), spasmodic torticollis, familial tremor, Gilles de la Tourette syndrome, syndromes of progressive ataxia, cerebellar degenerations, spinocerebellar degenerations, cerebellar cortical degeneration, olivopontocerebellar atrophy (OPCA), spinocerebellar degenerations, Friedreich's ataxia, spinal muscular atrophy, infantile spinal muscular atrophy (Werdnig-Hoffmann disease), juvenile spinal muscular atrophy (Wohlfart-Kugelberg-Welander disease), primary lateral sclerosis, hereditary spastic paraplegia, progressive neural muscular atrophy, progressive inflammatory neuropathy, polyneuropathies, mononeuritis multiplex, chronic familial polyneuropathies, hypertrophic interstitial polyneuropathy (Dejerine-Sottas disease), chronic inflammatory demyelinating polyradiculoneuropathy, polyneuropathy associated with anti-MAG IgM monoclonal gammopathy, post-herpetic neuralgia, Bannwarth syndrome, motor-predominant peripheral neuropathies, vestibular neuritis, olivopontocerebellar atrophy, Azorean (Machado-Joseph) disease, arthrogryposis multiplex congenita, progressive juvenile bulbar palsy, HTLV-1 associated myelopathy, progressive multifocal leukoencephalopathy, Creutzfeldt-Jakob disease, Gerstmann-Straussler-Scheinker disease, Korsakoff's disease, kuru, fatal familial insomnia, and Alper's disease.

In some embodiments, the patient is suffering from a neurodegenerative disease that involves the activation of microglia, recruitment and activation of macrophages, infiltration of inflammatory cells including myeloid cells that require BTK signaling to transmit activation signals, recognize integrins on activated endothelial cells, extravasate, or develop into cytokine and/or chemokine producing cells in situ. The inhibition of BTK further inhibits disease activity or disease progression by inhibiting neurodegenerative diseases associated with the toxic aggregation of protein, such as accumulation of beta amyloid deposits (amyloid plaque), neurofibrillary tangles, tau aggregation and hyper-phosphorylation, intracytoplasmic inclusion bodies, intracytoplasmic paired helical filaments, polyglucosan inclusions, Papp-Lantos bodies, ubiquitin-containing inclusions, and disorders where inadequate control of protein degradation and/or inability to dispose of mis-folded proteins leads to neurodegeneration. In some embodiments, the patient is suffering from a disease selected from the group consisting of sporadic and familial Alzheimer's disease, mild cognitive impairment, cerebral amyloid angiopathy, Lewy body dementia, Lewy body variant of Alzheimer's disease, Down's syndrome, Huntington's disease, striatonigral degeneration, multiple system atrophy (MSA-P, MSA-C, Shy-Drager syndrome), sporadic or hereditary amyotrophic lateral sclerosis (ALS or Lou Gehrig disease), primary lateral sclerosis, juvenile primary lateral sclerosis, neurodegenerative tauopathies, sporadic or hereditary synucleinopathies, neuronal intranuclear inclusion disease, Parkinson's disease, and frontotemporal dementia with Parkinsonism linked to chromosome 17 (FTDP-17).

In some embodiments, the patient is suffering from a neurodegenerative disorder wherein the inhibition of inflammatory processes in glial cells, myeloid cells, Schwann cells, oligodendrocytes and other myeloid-derived cell types resident in the CNS is accomplished through inhibition of signaling through the BTK pathway. In some embodiments, the patient is suffering from a disease selected from the group consisting of trinucleotide repeat disorders (polyglutamine diseases), Huntington's disease, spinocerebellar ataxia Types 1, 2, 3 (Machado-Joseph disease), 6, 7, and 17; spinal and bulbar muscular atrophy, Dentatorubral-pallidoluysian atrophy, neuronal ceroid lipofucsinoses, frontotemporal dementia (Pick's disease, primary progressive aphasia, and semantic dementia), corticobasal degeneration, and progressive supranuclear palsy.

In some embodiments, the patient is suffering from a disease selected from the group consisting of sporadic or hereditary prion disease, prion-disorders such as Creutzfeldt-Jakob disease, kuru, Gerstmann-Straussler-Scheinker syndrome, and disorders leading to olivopontocerebellar atrophy, sporadic fatal insomnia, and fatal familial insomnia.

In some embodiments, the patient is suffering from a neuroinflammatory disorder which results from CNS ischemia. In some embodiments, the patient is suffering from an ischemic event, or neuroinflammatory and neurodegenerative disorders associated with ischemic brain injury, including vascular dementia, mild cognitive impairment, cerebrovascular accident, stroke, transient ischemic attack (mini-stroke), focal brain ischemia, multifocal brain ischemia, thrombotic stroke, embolic stroke, and the development of an infarct or penumbra around an area of restricted or constrained blood flow.

In some embodiments, the patient is suffering from an autoimmune mediated neurodegenerative disorder in the central and/or peripheral nervous system. In some embodiments, the patient is suffering from a disease selected from the group consisting of neuromyelitis optica (Devic's syndrome), Guillain-Barre syndrome, multiple sclerosis, clinically isolated syndrome, relapsing-remitting multiple sclerosis, malignant multiple sclerosis, primary progressive multiple sclerosis, neuromyelitis optica spectrum diseases, Balo concentric sclerosis, Marburg multiple sclerosis, diffuse myelinoclastic sclerosis, chronic focal encephalitis, Rasmussen's encephalitis, stiff person syndrome, myasthenia gravis, polyneuropathy associated with anti-MAG IgM monoclonal gammopathy.

In some embodiments, the patient is suffering from polyneuropathies resulting from infection or post-infection neuroinflammation, including Bannworth syndrome (Lyme disease), chronic encephalomyelitis (Lyme disease), post-herpetic neuralgia, HTLV-1 associated myelopathy; progressive multifocal leukoencephalopathy; chronic fatigue syndrome (CFS), systemic exertion intolerance disease (SEID), myalgic encephalomyelitis (ME), post-viral fatigue syndrome (PVFS), chronic fatigue immune dysfunction syndrome (CFIDS), Meniere's disease (vertigo-inner ear endolymph fluid regulation), Guillain-Barre syndrome, amyotrophic lateral sclerosis, progressive bulbar palsy, infantile progressive bulbar palsy (or juvenile progressive bulbar palsy), Bell's palsy, vestibular neuritis, acute disseminated encephalomyelitis, recurrent or multiphasic disseminated encephalomyelitis, and chronic encephalomyelitis.

In some embodiments, the patient is suffering from a heritable neurodegenerative disorder wherein a genetic mutation results in degeneration in peripheral or central nerves, spinal nerves, dorsal root ganglia or particularly in the myelin sheath protecting these structures; and/or causes inflammatory responses secondary to defects of the neurons, Schwann cells, glial cells or astrocytes. In some embodiments, the patient is suffering from a disease selected from the group consisting of Charcot-Marie-Tooth disease, Dejerine-Sottas disease, hypertrophic interstitial neuropathy, Rett syndrome, lysosomal storage diseases and/or lipid storage disorders (Gaucher disease, Tay-Sachs disease, Neimann-Pick disease Types A, B and C, Farber's disease, GM1 gangliosidosis, GM2 gangliosidosis, mucopolysaccharidoses type I (including Hurler, Hurler-Scheie, and Scheie syndromes), neuronal ceroid lipofucsinoses (Santavuori-Haltia disease, Jansky-Bielschowsky disease, Batten disease, Kufs disease, and other childhood/juvenile neuronal ceroid lipofucsinoses), leukodystrophies (including adrenoleukodystrophy, metachromatic leukodystrophy, Canavan disease, Alexander disease, Pelizaeus-Merzbacher disease); and mitochrondrial dysfunctions such as Friedreich's ataxia chronic progressive external ophthalmoplegia, Alper's disease, spinal muscular atrophy (inherited SMN1 or SMN2 mutation), infantile spinal muscular atrophy (Werdnig-Hoffman disease), juvenile spinal muscular atrophy (Wohlfart-Kugelberg-Welander disease), arthrogryposis multiplex congenita, and diseases in which inflammation may lead to loss of motor nerves (especially long nerves) such as hereditary spastic paraplegia.

In some embodiments, the patient is suffering from asthma. As used herein, “asthma” encompasses airway constriction regardless of the cause, including reactive airway disease. Common triggers of asthma include, but are not limited to, exposure to an environmental stimulants (e.g., allergens), cold air, warm air, perfume, moist air, exercise or exertion, and emotional stress. Also provided herein is a method of treating, preventing and/or managing one or more symptoms associated with asthma. Examples of the symptoms include, but are not limited to, severe coughing, airway constriction, and mucus production.

In some embodiments, the patient is suffering from a solid tumor cancer wherein the dose of the BTK inhibitor administered is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In selected embodiments, the invention relates to a method of treating pancreatic cancer, breast cancer, ovarian cancer, melanoma, lung cancer, head and neck cancer, and colorectal cancer using a BTK inhibitor, wherein the dose is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts.

The amounts of the BTK inhibitors administered to a patient suffering from a BTK mediated disorder will be dependent on the mammal being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In selected embodiments, the BTK inhibitor is administered in a single dose. Typically, such administration will be by injection, for example by intravenous injection, in order to introduce the agents quickly. However, other routes may be used as appropriate. A single dose of the BTK inhibitor may also be used for treatment of an acute condition.

In selected embodiments, the BTK inhibitor is administered in multiple doses. Dosing may be about once, twice, three times, four times, five times, six times, or more than six times per day. Dosing may be about once a month, once every two weeks, once a week, or once every other day. In other embodiments, the BTK inhibitor is administered about once per day to about 6 times per day. In another embodiment the administration of the BTK inhibitor continues for less than about 7 days. In yet another embodiment the administration continues for more than about 6, 10, 14, 28 days, two months, six months, or one year. In some cases, continuous dosing is achieved and maintained as long as necessary.

Administration of the agents of the invention may continue as long as necessary. In selected embodiments, the BTK inhibitor is administered for more than 1, 2, 3, 4, 5, 6, 7, 14, or 28 days. In some embodiments, the BTK inhibitor is administered for less than 28, 14, 7, 6, 5, 4, 3, 2, or 1 day. In selected embodiments, the BTK inhibitor is administered chronically on an ongoing basis—e.g., for the treatment of chronic effects.

An effective amount of the combination of the BTK inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein. Reagents described in the examples are commercially available or may be prepared according to procedures described in the literature.

### Example 1. General Synthesis of BTK Probes

The BTK probes of the present invention can be prepared by methods well known in the art of organic chemistry. See, for example, March, Advanced Organic Chemistry, 4th Edition, John Wiley & Sons, 2001. During synthetic processes it may be necessary and/or desirable to protect sensitive or reactive groups on any of the molecules concerned. This is achieved by means of conventional protecting groups, such as those described in Greene and Wutts, Protective Groups in Organic Synthesis, 3rd Edition, John Wiley & Sons, 1999. The protective groups are optionally removed at a convenient subsequent stage using methods well known in the art.

The products of the reactions are optionally isolated and purified, if desired, using conventional techniques, but not limited to, filtration, distillation, crystallization, chromatography and the like. Such materials are optionally characterized using conventional means, including the measurement of physical constants and spectral data.

BTK probes included in the present invention may be synthesized by the following routes. Boronic acid pinacol esters may be prepared as follows:

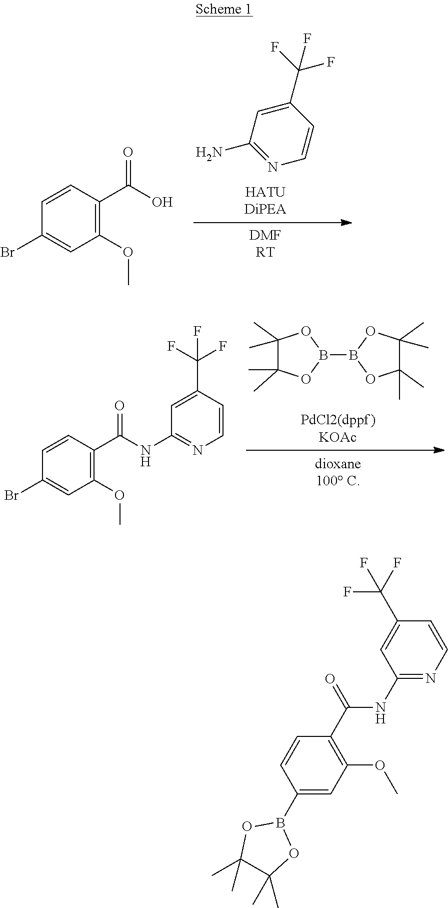

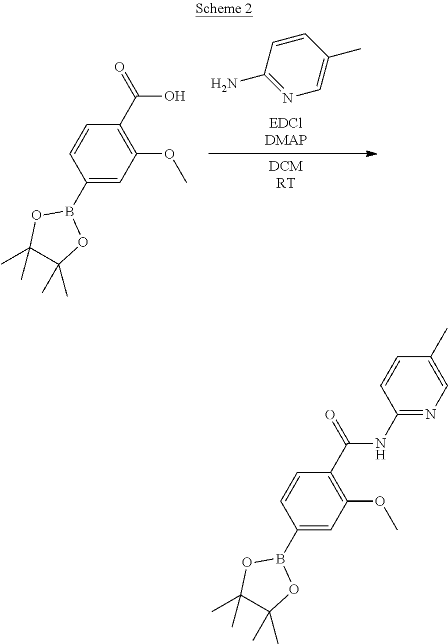

The following compound may be prepared in an analogous manner to the preparations shown in Scheme 1 and Scheme 2:

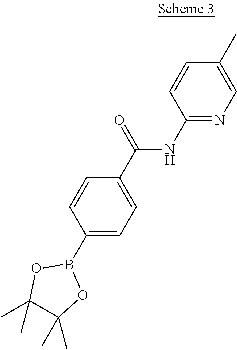

Additional boronic acid pinacol esters may be prepared as follows:

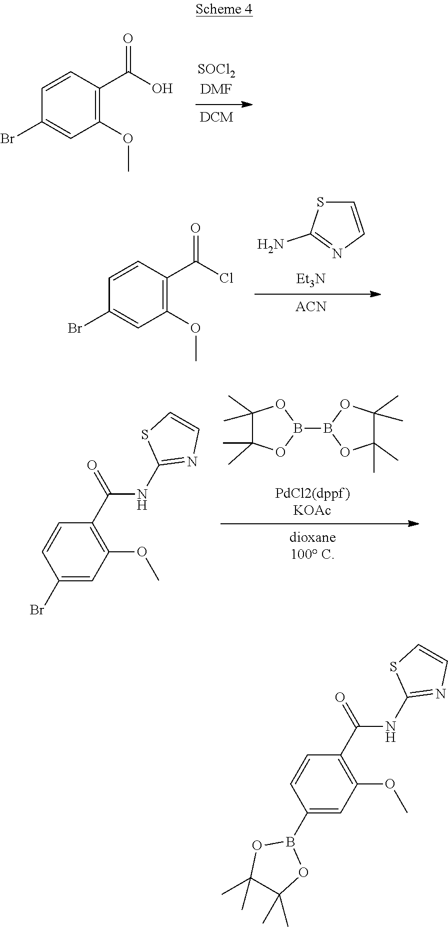

Boronic acids may be prepared as follows:

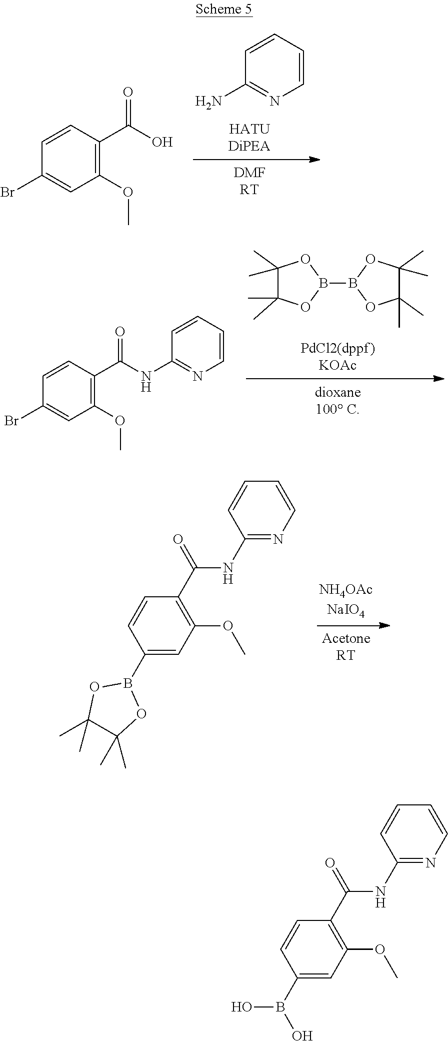

Pyrollidine derivatives may be prepared as follows (wherein CBz refers to carboxybenzyl):

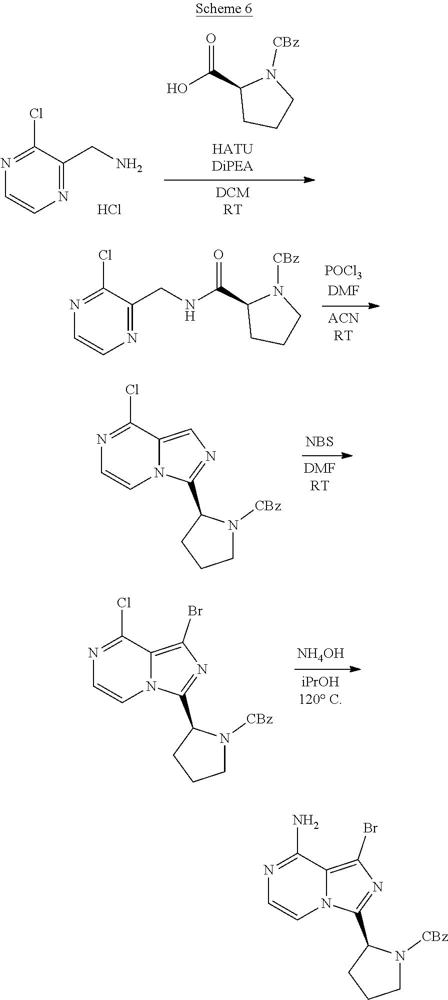

CBz-protected alanine and N-methylalanine derivatives are prepared in an analogous manner.

Pyrollidine derivatives may be also prepared as follows:

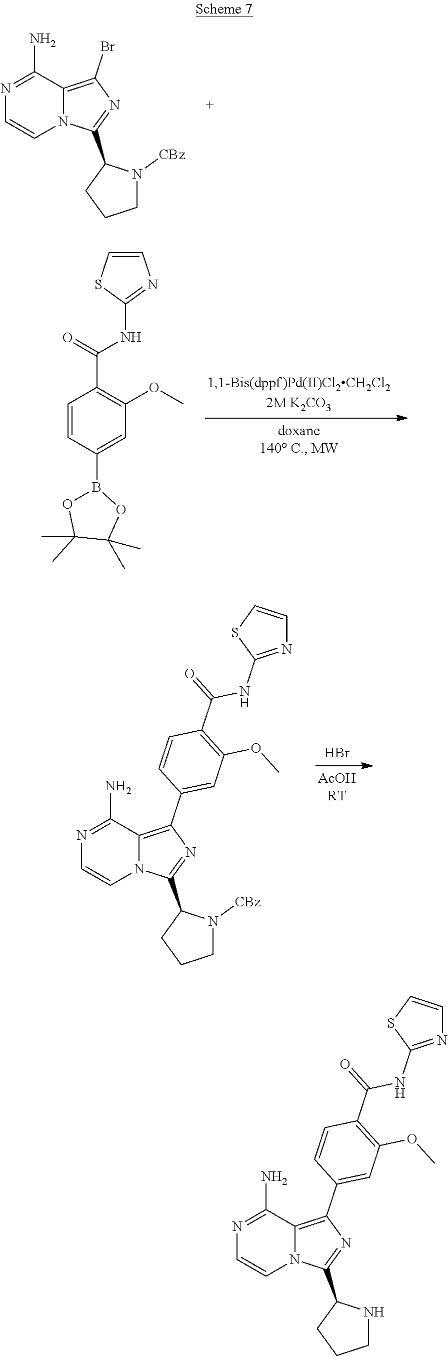

In the aforementioned syntheses, boronic acids and boronic acid pinacol esters perform equally well in the Suzuki coupling step. Alternative synthetic schemes can be used, such as those described in U.S. Patent Application Publication No. 2014/0155385 A1, which are incorporated by reference herein.

The final compound of Scheme 7 may be functionalized at the amino position of the pyrrolidine ring to attach functional groups capable of covalent binding to BTK and which also provide tags suitable for detection using fluorescence, chemiluminescence, and/or electrochemiluminescence based methods. Example tags include:

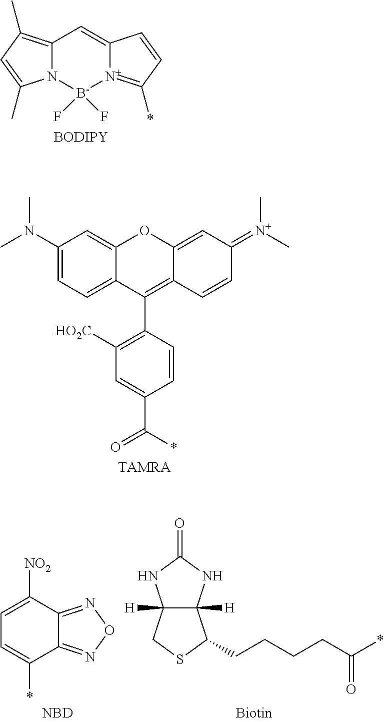

In one embodiment, the tag is a member of Alexa Fluor family of fluorescent dyes, including AF647, AF350, AF405, AF430, AF488, AF514, AF532, AF546, AF568, AF594, and AF610. Some of the chemical structures of the Alexa Fluor (AF) dyes are shown below.

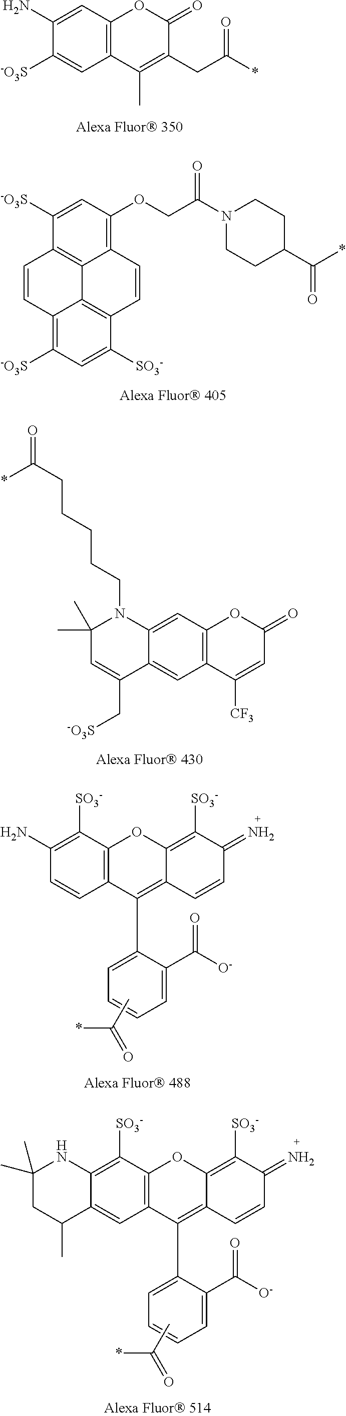

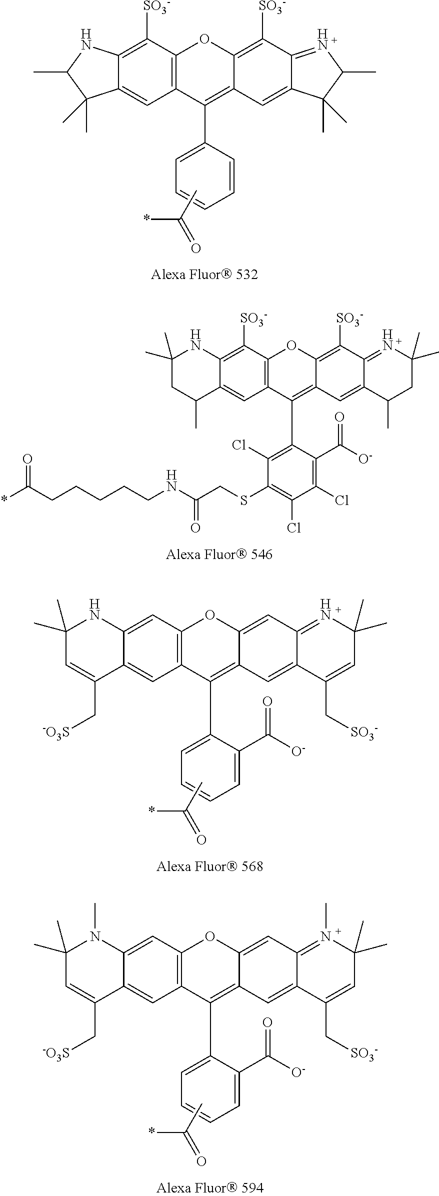

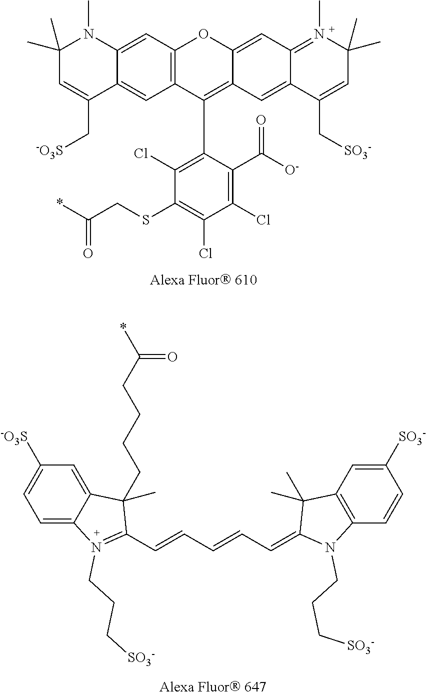

Other suitable tags are known to those of ordinary skill in the art, and include those tags described in Cravatt, et al., Annu. Rev. Biochem. 2008, 77, 383-414. Groups may be attached by amine or amide linkers using coupling methods known to those of ordinary skill in the art.

The present invention also includes within its scope all stereoisomeric forms of the BTK probes according to the present invention resulting, for example, because of configurational or geometrical isomerism. Such stereoisomeric forms include enantiomers, diastereoisomers, cis and trans isomers, etc. In the case of the individual stereoisomers of compounds described herein, the present invention also includes the aforementioned stereoisomers substantially free, i.e., associated with less than 5%, preferably less than 2% and in particular less than 1% of the other stereoisomer. Mixtures of stereoisomers in any proportion, for example a racemic mixture comprising substantially equal amounts of two enantiomers are also included within the scope of the present invention.

For chiral compounds, methods for asymmetric synthesis whereby the pure stereoisomers are obtained are well known in the art, e.g. synthesis with chiral induction, synthesis starting from chiral intermediates, enantioselective enzymatic conversions, separation of stereoisomers using chromatography on chiral media. Such methods are described in Collins, et al., eds., Chirality in Industry, John Wiley & Sons, 1992. Likewise, methods for synthesis of geometrical isomers are also well known in the art.

The compounds of the present invention, which can be in the form of a free base, may be isolated from the reaction mixture in the form of a pharmaceutically acceptable salt. The pharmaceutically acceptable salts may also be obtained by treating the free base of the BTK inhibitors disclosed herein with an organic or inorganic acid such as hydrogen chloride, hydrogen bromide, hydrogen iodide, sulfuric acid, phosphoric acid, acetic acid, propionic acid, glycolic acid, maleic acid, malonic acid, methanesulphonic acid, fumaric acid, succinic acid, tartaric acid, citric acid, benzoic acid, and ascorbic acid.

The compounds of the present invention disclosed herein may also exist as amorphous forms or as multiple crystalline forms, also known as polymorphic forms, and as salts, solvates (including hydrates), and cocrystals. All physical forms, including all crystalline and amorphous phases, are included within the scope of the present invention. A typical, non-limiting, process for the preparation of a crystalline form or solvate involves dissolving the inventive compound in desired amounts of the desired solvent (organic or water or mixtures thereof) at a higher than ambient temperature, and cooling the solution at a rate sufficient to form crystals which are then isolated by standard methods.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁷O, ¹⁸O, ¹⁸F, ³²P, ³⁵S, and ³⁶Cl, respectively.

Radioisotopically-labelled forms of the compounds disclosed herein (e.g., those labeled with ³H and ¹⁴C) are useful in compound and/or substrate tissue distribution assays. Tritium (³H) and carbon-14 (¹⁴C) isotopes are particularly preferred for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Isotopically-labelled forms of the compounds disclosed herein can generally be prepared by following procedures analogous to those disclosed in the Schemes and/or in the Examples described below, by substituting an appropriate isotopically labeled reagent for a non-isotoplically labeled reagent.

### Example 2. Analytical Methods

The following high performance liquid chromatography (HPLC) and LC mass spectrometry (LCMS) methods may be used to characterize compounds included in the present invention.

### LC-MS Method

### Agilent Mass Spectrometer

### Detector: DAD (210, 254 and 280 nm)

Mass detector: atmospheric pressure ionization-electrospray (API-ES) (10-2000 amu, pos./neg. ion mode)
Eluents (mobile phase): A: 0.1% formic acid in MilliQ-water, B: acetonitrile

### Column: Waters XTerra C18 MS, 50×4.6 mm ID, 2.5 μm

Flow rate: 0.5 mL/min

### Gradient Elution Program:

| Time (min) | A (%) | B (%) |
|---|---|---|
| 0.0 | 90 | 10 |
| 7.0 | 10 | 90 |
| 7.1 | 0 | 100 |
| 10.0 | 90 | 10 |

### HPLC Method

### Gilson Analytical HPLC System

### Column: Phenomenex Luna C18(2) (100×2.00 mm, 5 μm)

### Detector: UV/Vis (210/240 nm)

Flow rate: 1 mL/min
Eluents (mobile phase): A: acetonitrile, B: acetonitrile/MilliQ-water=1/9 (v/v), C: 0.1% TFA in MilliQ-water.

### Gradient Elution Program:

| Time (min) | A (%) | B (%) | C (%) |
|---|---|---|---|
| 0.00 | 0 | 97 | 3 |
| 11.90 | 97 | 0 | 3 |
| 14.40 | 97 | 0 | 3 |
| 15.40 | 0 | 97 | 3 |

Retention times are reported as “Rt” in the following examples.

### Example 3. Preparation of BTK Probe N-[3-[2-[2-[3-[5-[(3aS,4S,6aR)-2-oxo-1,3,3a,4,6,6a-hexahydrothieno[3,4-d]imidazol-4-yl]pentanoylamino]propoxy]ethoxy]ethoxy]propyl]-N′-[2-[4-[(E)-4-[(2S)-2-[8-amino-1-[4-(2-pyridylcarbamoyl)phenyl]imidazo[1,5-a]pyrazin-3-yl]-1-piperidyl]-4-oxo-but-2-enyl]piperazin-1-yl]ethyl]pentanediamide) (Formula (3))

A stepwise approach described in this example was used to prepare the title compound. Other methods of preparation will be apparent to the ordinarily skilled artisan.

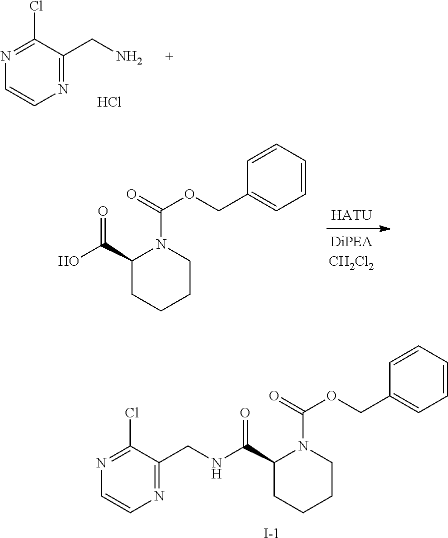

2-Chloro-3-aminomethylpyrazine HCl (2 g; 11.1 mmol), (S)-(−)-1-(carbobenzyloxy)-2-piperidine carboxylic acid (3.2 g; 12.2 mmol) and N,N-diisopropylethylamine (7.7 mL; 44.4 mmol) were dissolved in dichloromethane (100 mL). N-[(dimethylamino)-1H-1,2,3-triazolo-[4,5-b]pyridin-1-ylmethylene]-N-methylmethanaminium hexafluorophosphate N-oxide (HATU) (6.3 g; 16.7 mmol) was added and the resulting mixture was stirred overnight at room temperature. The mixture was washed with aqueous sodium bicarbonate solution and water. The organic layer was then dried over sodium sulfate, filtered and evaporated to dryness. The crude material was chromatographed over SiO₂ using a gradient of 20-80% ethyl acetate in heptane to give 4.4 g; 11.3 mmol of benzyl (2S)-2-[(3-chloropyrazin-2-yl)methylcarbamoyl]piperidine-1-carboxylate (I-1) as a colorless oil (73%). Data: LCMS Rt=5.85 min; m/z 389.2 (M+H)⁺; HPLC Rt=8.30 min; ¹H NMR (400 MHz, CDCl₃, 300 K): δ=8.35 (1H, d), 8.29 (1H, d), 7.35 (5H, bs), 5.20 (2H, s), 4.95 (1H, s), 4.76 (1H, d), 4.60 (1H, dd), 4.18 (1H, bs), 3.03 (1H, bs), 2.36 (1H, bd), 1.88-1.47 (6H, m).

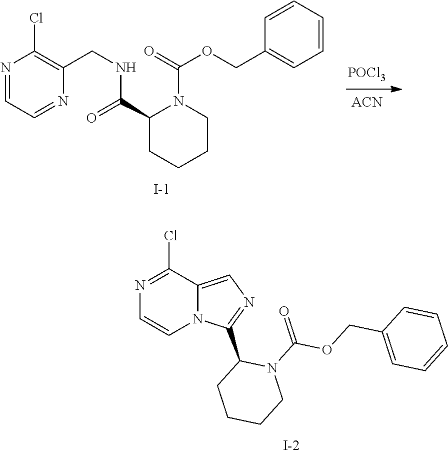

Benzyl (2S)-2-[(3-chloropyrazin-2-yl)methylcarbamoyl]piperidine-1-carboxylate (4.4 g; 11.3 mmol) was dissolved in acetonitrile (100 mL). Phosphorus oxychloride (3.2 mL; 34.0 mmol) was added dropwise, followed by DMF (88 μL; 1.1 mmol). The mixture was stirred at 20° C. overnight, and then diluted with DCM (250 mL). Aq. sodium bicarbonate solution (100 mL) was added slowly and the mixture was stirred until gas evolution subsided. The organic layer was dried on sodium sulfate, filtered and evaporated to dryness. Flash SiO₂ chromatography using a gradient of 0-50% of EtOAc in heptane yielded 2.3 g; 6.2 mmol of benzyl (2S)-2-(8-chloroimidazo[1,5-a]pyrazin-3-yl)piperidine-1-carboxylate (I-2) as a light yellow oil (55%). Data: LCMS Rt=6.94 min; m/z 371.1 (M+H)⁺; HPLC Rt=9.92 min; ¹H NMR (400 MHz, CDCl₃, 300 K): δ=7.95 (1H, bs), 7.79 (1H, s), 7.36 (5H, m), 7.19 (1H, s), 5.82 (1H, s), 5.19 (2H, m), 4.01 (1H, d, J=13.1), 2.70 (1H, dt, J1=13.1, J2=2.8), 2.42 (2H, m), 1.99 (1H, m), 1.83 (1H, d, J=15.3), 1.71 (1H, d, J=15.3), 1.56 (1H, m).

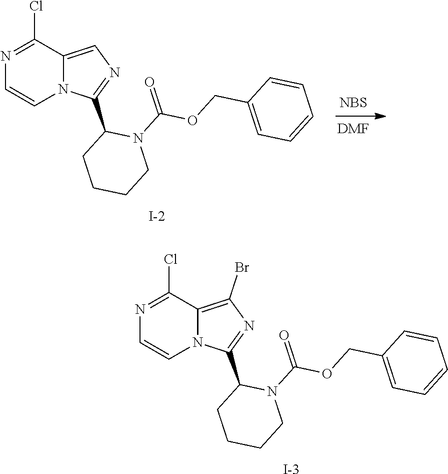

Benzyl-(2S)-2-(8-chloroimidazo[1,5-a]pyrazin-3-yl)piperidine-1-carboxylate (2.3 g; 6.2 mmol) was dissolved in DMF (25 mL). N-Bromosuccinimide (1.2 g; 6.8 mmol) was added under stirring. The mixture was stirred at room temperature for 4 hours. DCM (200 mL) and aqueous sodium bicarbonate solution (100 mL) were added to the mixture, and the layers were separated. The organic layer was dried over sodium sulfate, filtered and evaporated to dryness. Flash SiO₂ chromatography using a gradient of 0-100% of EtOAc in heptane yielded 2.7 g; 6.0 mmol of benzyl-(2S)-2-(1-bromo-8-chloro-imidazo[1,5-a]pyrazin-3-yl)piperidine-1-carboxylate (I-3) as a beige solid (96%). Data: LCMS Rt=7.69 min; m/z 450.1 (M+H)⁺; HPLC Rt=11.18 min; ¹H NMR (400 MHz, CDCl₃, 300 K): δ=7.94 (1H, bs), 7.36 (5H, m), 7.17 (1H, s), 5.76 (1H, s), 5.19 (2H, m), 4.00 (1H, d, J=13.6), 2.73 (1H, dt, J1=13.2, J2=2.8), 2.34 (2H, m), 1.96 (1H, m), 1.80 (1H, d, J=13.2), 1.72 (1H, d, J=13.2), 1.54 (1H, m).

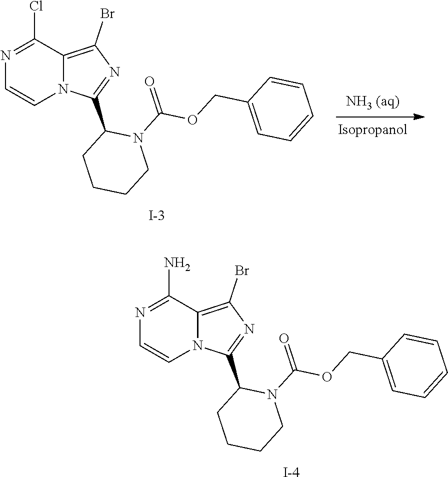

Benzyl-(2S)-2-(1-bromo-8-chloro-imidazo[1,5-a]pyrazin-3-yl)piperidine-1-carboxylate (2.7 g; 6.0 mmol) was suspended in isopropanol (20 mL) and aqueous ammonia (20 mL), and transferred into two microwave vials (20 mL max. capacity). The vials were capped and heated to 125° C. for a total of 2.5 hours each. The mixture was evaporated to dryness. The crude product was purified by flash SiO₂ chromatography using a gradient of 0-5% of MeOH in DCM to give 2.2 g; 5.0 mmol of benzyl-(2S)-2-(8-amino-1-bromo-imidazo[1,5-a]pyrazin-3-yl)piperidine-1-carboxylate (I-4) as a yellow solid (83%). Data: LCMS Rt=4.70 min; m/z 431.1 (M+H)⁺; HPLC Rt=6.27 min; ¹H NMR (400 MHz, dimethylsulfoxide-d₆ (DMSO-d₆), 300K): δ=7.47 (1H, bd), 7.32 (5H, m), 6.95 (1H, s), 6.71 (2H, s), 5.68 (1H, m), 5.13 (2H, m), 3.90 (1H, m), 3.04 (1H, bt), 1.94 (3H, m), 1.65 (2H, m), 1.44 (1H, m).

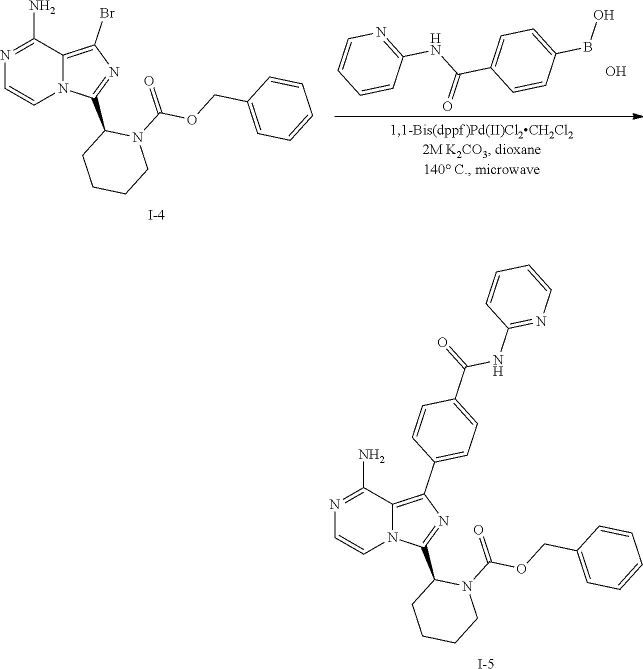

Benzyl-(2S)-2-(8-amino-1-bromo-imidazo[1,5-a]pyrazin-3-yl)piperidine-1-carboxylate (1 g; 2.3 mmol) and 4-(pyridine-2-yl)aminocarbonylphenylboronic acid (562 mg; 2.3 mmol) were dissolved in dioxane (16 mL). 2 M potassium carbonate solution in water (4 mL) was added. The mixture was purged with N₂ for 5 minutes, after which 1,1′-bis(diphenylphosphino)ferrocene]dichloropalladium(II), complex with dichloromethane (95 mg; 0.12 mmol) was added. The resulting mixture was heated to 140° C. for 25 minutes. The mixture was diluted with DCM (50 mL) and washed with water (25 mL). The organic layer was dried over sodium sulfate, filtered and evaporated to dryness to give a light brown oil. The crude product was purified by flash SiO₂ chromatography using a gradient of 0-5% of MeOH in DCM to give 1.2 g; 2.1 mmol of benzyl (2S)-2-[8-amino-1-[4-(2-pyridylcarbamoyl)phenyl]imidazo[1,5-a]pyrazin-3-yl]piperidine-1-carboxylate (I-5) as a yellow oil (91%). Data: LCMS Rt=5.04 min; m/z 548.2 (M+H)⁺; HPLC Rt=6.86 min; ¹H NMR (400 MHz, DMSO-d₆, 300 K): δ=10.86 (1H, s), 8.41 (1H, m), 8.24 (1H, d, J=8.3), 8.17 (2H, d, J=8.5), 7.86 (1H, m), 7.76 (2H, d, J=8.4), 7.54 (1H, ds), 7.31 (4H, bs), 7.19 (1H, m), 7.05 (1H, bs), 6.21 (2H, s), 5.79 (1H, d, J=5.0), 5.15 (2H, m), 3.95 (1H, d, J=13.6), 3.18 (1H, bs), 2.14 (2H, bs), 1.90 (1H, m), 1.70 (2H, m), 1.49 (1H, m).

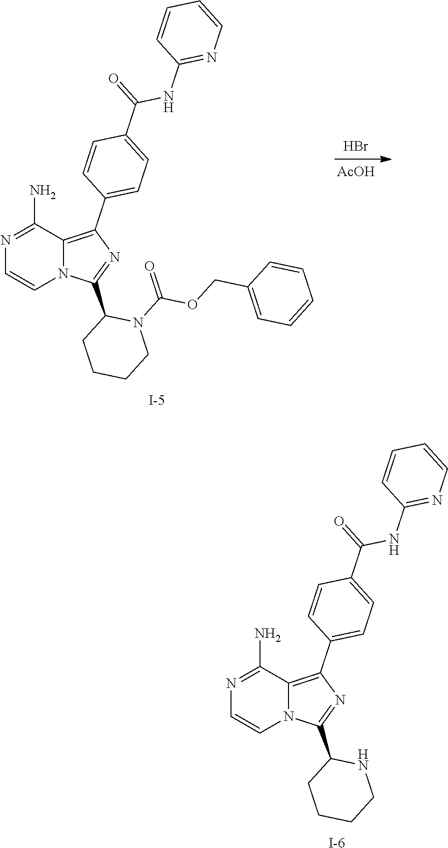

Benzyl (2S)-2-[8-amino-1-[4-(2-pyridylcarbamoyl)phenyl]imidazo[1,5-a]pyrazin-3-yl]piperidine-1-carboxylate (1.2 g; 2.1 mmol) was dissolved in 33% HBr in acetic acid (20 mL) and held at room temperature overnight. The mixture was diluted with water (150 mL) and washed with dichloromethane (100 mL). The aqueous layer was made basic using 2 N aqueous sodium hydroxide solution, and then extracted with dichloromethane (200 mL). The organic layer was dried over sodium sulfate, filtered and evaporated to dryness to give 715 mg; 1.7 mmol of 4-[8-amino-3-[(2S)-2-piperidyl]imidazo[1,5-a]pyrazin-1-yl]-N-(2-pyridyl)benzamide (I-6) as a yellow solid (79%). Data: LCMS Rt=1.70 min; m/z 414.2 (M+H)⁺; HPLC Rt=0.47 min; ¹H NMR (400 MHz, DMSO-d₆, 300 K): δ=10.84 (1H, s), 8.42 (1H, m), 8.23 (1H, d, J=8.3), 8.16 (2H, d, J=8.5), 7.93 (1H, d, J=4.9), 7.85 (1H, m), 7.75 (2H, d, J=8.5), 7.18 (1H, m), 7.07 (1H, d, J=4.9), 6.12 (2H, bs), 4.14 (1H, m), 3.00 (1H, d, J=11.7), 2.69 (2H, t, J=11.0), 1.88 (3H, m), 1.54 (3H, m).

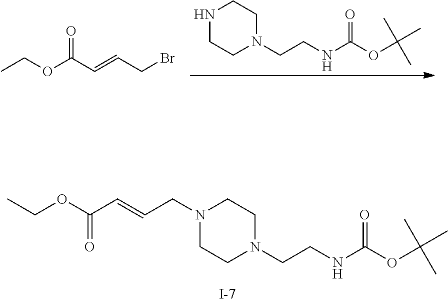

A mixture of tert-butyl-N-(2-piperazin-1-ylethyl)carbamate (1 g; 4.4 mmol), potassium carbonate (1.2 g, 8.7 mmol) and ethyl (E)-4-bromobut-2-enoate (674 μL, 4.9 mmol) in ethanol (15 mL) was stirred at room temperature for 3 hours. The mixture was diluted with ethyl acetate (20 mL) and washed with water (20 mL). The organic layer was dried over sodium sulfate, filtered and evaporated to dryness to give a dark brown oil. The crude product was purified by silica column chromatography (0 to 10% methanol in dichloromethane) to give 895 mg; 2.6 mmol of ethyl (E)-4-[4-[2-(tert-butoxycarbonylamino)ethyl]piperazin-1-yl]but-2-enoate (I-7) as a yellow oil (60%). Data: LCMS Rt=3.67 min; m/z 342.3 (M+H)⁺. ¹H NMR (400 MHz, DMSO-d₆, 300K): δ=6.78 (1H, dt, J1=15.7, J2=5.9), 6.62 (1H, t, J=5.3), 5.99 (1H, dt, J1=15.7, J2=1.6), 4.11 (2H, q, J=7.1), 3.09 (2H, dd, J1=6.0, J=1.6), 3.01 (2H, m), 2.31 (9H, m), 1.37 (9H, s), 1.21 (3H, t, J=7.1).

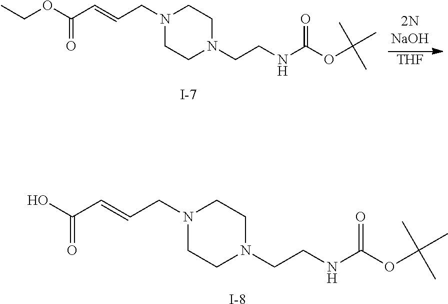

A mixture of potassium hydroxide (2 mL, 4 mmol) ethyl (E)-4-[4-[2-(tert-butoxycarbonylamino)ethyl]piperazin-1-yl]but-2-enoate (I-7, 673 μL, 2.6 mmol) in tetrahydrofuran (20 mL) was stirred at room temperature for 3 hours. Mainly starting material, some desired product was observed. Potassium hydroxide (2 mL, 4 mmol) was added and the reaction mixture was stirred overnight at room temperature. Still some starting material left. 6N HCl (1.3 mL) was added and the reaction mixture was concentrated under reduced pressure. Then, 10 mL of methanol was added. The white precipitate was filtered off and the filtrate was concentrated under reduced pressure to give 910 mg; 2.4 mmol of (E)-4-[4-[2-(tert-butoxycarbonylamino)ethyl]piperazin-1-yl]but-2-enoic acid (I-8) as a light brown solid (93%). Data: LCMS Rt=2.60 min; m/z 314.3 (M+H)⁺. ¹H NMR (400 MHz, DMSO-d₆, 300 K): δ=6.73 (1H, dt, J1=15.7, J2=6.1), 6.65 (1H, t, J=5.3), 5.3 (1H, d, J=15.7), 3.08 (2H, dd, J=6.1), 3.02 (2H, m), 2.315 (9H, m), 1.37 (9H, s).

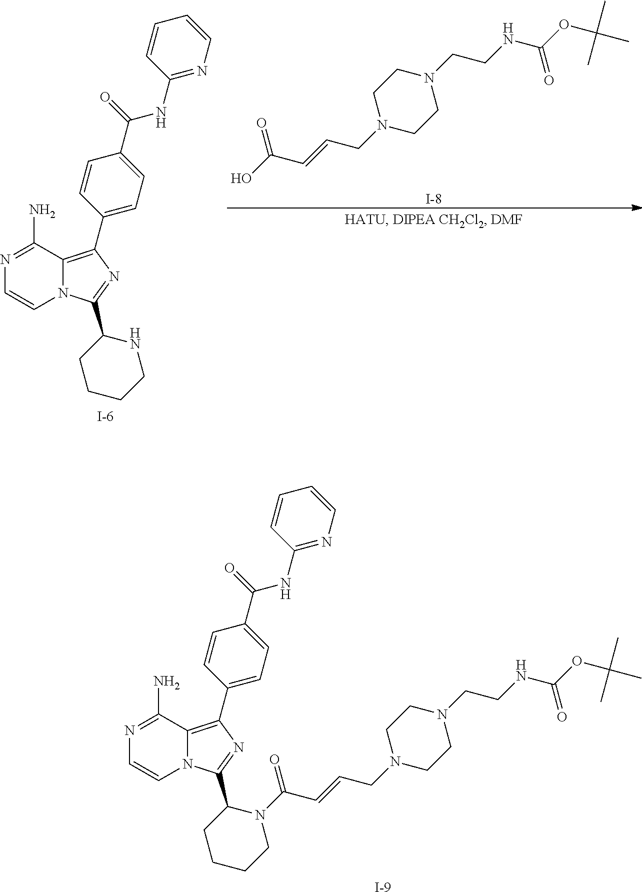

A solution of 4-[8-amino-3-[(2S)-2-piperidyl]imidazo[1,5-a]pyrazin-1-yl]-N-(2-pyridyl)benzamide I-6 (250 mg; 0.60 mmol), (E)-4-[4-[2-(tert-butoxycarbonylamino)ethyl]piperazin-1-yl]but-2-enoic acid I-8 (271 mg; 0.73 mmol), HATU (345 mg; 0.91 mmol) and N,N-diisopropylethylamine (400 μL; 2.41 mmol) in dichloromethane (10 mL) was stirred at room temperature for 2 hours. Water (10 mL) was added to the mixture and stirred for 10 minutes. The organic layer was dried over sodium sulfate, filtered and evaporated to dryness to give a yellow oil. The crude product was purified by silica column chromatography (0 to 10% methanol in dichloromethane) to give 262 mg; 0.37 mmol of tert-butyl-N-[2-[4-[(E)-4-[(2S)-2-[8-amino-1-[4-(2-pyridylcarbamoyl)phenyl]imidazo[1,5-a]pyrazin-3-yl]-1-piperidyl]-4-oxo-but-2-enyl]piperazin-1-yl]ethyl]carbamate (I-9) as a brown oil (61%). Data: LCMS Rt=3.92 min; m/z 709.4 (M+H)⁺; 707.3 (M+H)⁻. ¹H NMR (400 MHz, DMSO-d₆, 300 K): δ=10.85 (1H, s), 8.41 (1H, m), 8.23 (1H, m), 8.16 (2H, m), 7.86 (1H, m), 7.77 (2H, d, J=8.2), 7.55 (1H, d, J=4.9), 7.19 (1H, m), 7.12 (1H, d, J=4.9), 6.70-6.59 (2H, m), 6.21 (2H, s), 3.90 (1H, s, br), 3.49 (1H, s, br), 3.20-2.94 (4H, m), 2.47-2.17 (10H, m), 1.91-1.64 (3H, m), 1.49 (1H, m), 1.37 (9H, s).

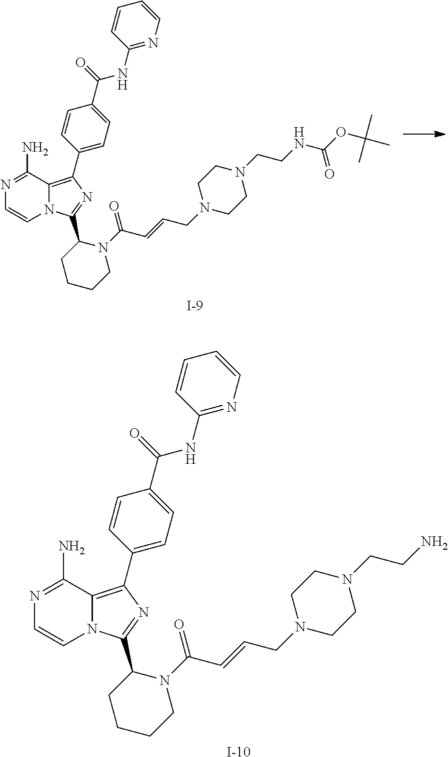

Tert-butyl-N-[2-[4-[(E)-4-[(2S)-2-[8-amino-1-[4-(2-pyridylcarbamoyl)phenyl]imidazo[15-a]pyrazin-3-yl]-1-piperidyl]-4-oxo-but-2-enyl]piperazin-1-yl]ethyl]carbamate I-9 (260 mg; 0.37 mmol) was dissolved in dichloromethane (20 mL) and trifluoroacetic acid (5 mL) and stirred at room temperature for 2 hours. Aqueous 2N sodium hydroxide was added to the mixture until neutral. The organic layer was dried over sodium sulfate, filtered and evaporated to dryness to give 175 mg; 0.29 mmol of 4-[8-amino-3-[(2S)-1-[(E)-4-[4-(2-aminoethyl)piperazin-1-yl]but-2-enoyl]-2-piperidyl]imidazo[1,5-a]pyrazin-1-yl]-N-(2-pyridyl)benzamide (I-10) as a yellow solid (78% crude). LCMS Rt=2.76 min; m/z 609.4 (M+H)⁺; HPLC Rt=0.98 min; ¹H NMR (400 MHz, DMSO-d₆, 300 K): δ=8.41 (1H, m), 8.23 (1H, dt, J=8.3), 8.17 (2H, d, J=8.5), 7.86 (1H, m), 7.78 (2H, d, J=7.2), 7.56 (1H, d, J=4.2), 7.18 (1H, t), 7.12 (1H, d, J=4.8), 6.67 (2H, bs), 6.20 (2H, bs), 3.91 (1H, m), 3.23-3.09 (4H, m), 2.61 (1H, t, J=7.0), 2.37 (12H, m), 1.87-1.64 (4H, m), 1.47 (1H, t).

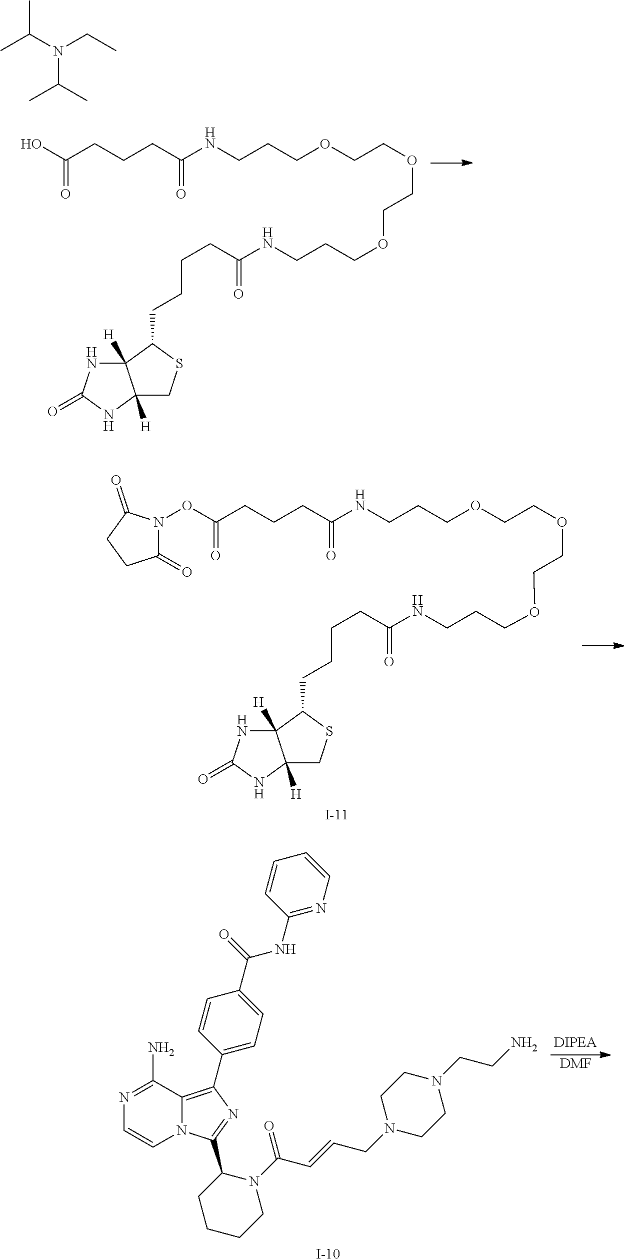

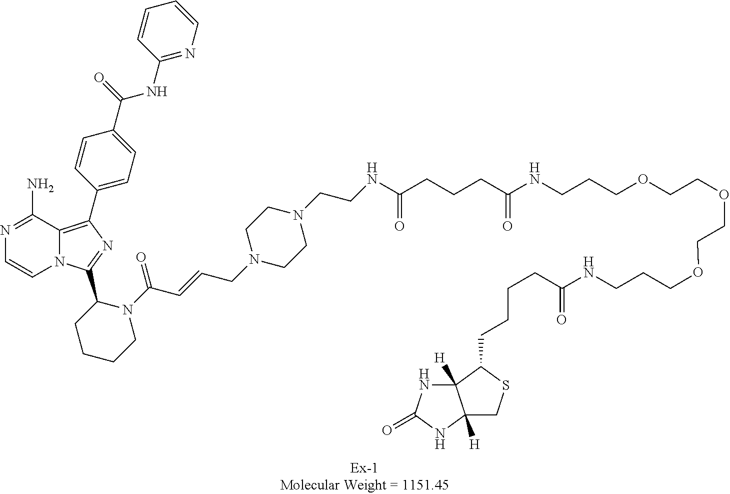 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12]

N-biotinyl-NH(PEG)2-COOH (242 mg; 0.351 mmol) was dissolved in DMF (8 mL) under a nitrogen atmosphere. N-hydroxysuccinimide (48.4 mg; 0.421 mmol) and N-ethyl-N-(3-dimethylaminopropyl)-carboiimide hydrochloride (80.6 mg; 0.421 mmol) were added and the reaction mixture was stirred at room temperature overnight.

The reaction mixture was added dropwise to a solution of 4-[8-amino-3-[(2S)-1-[(E)-4-[4-(2-aminoethyl)piperazin-1-yl]but-2-enoyl]-2-piperidyl]imidazo[1,5-a]pyrazin-1-yl]-N-(2-pyridyl)benzamide (I-10) (175.1 mg; 0.288 mmol) and DIPEA (75.4 μL) in DMF (4 mL) at −10° C. The reaction mixture was allowed to come to room temperature overnight, concentrated to half the volume under reduced pressure and then purified by preparative HPLC (column: Luna C-18, eluent 0-40% ACN in water+0.5% TFA). The pure fractions were collected and converted to the free base using an SCX column (eluent: MeOH/DiPEA 9/1). The resulting solution was concentrated under reduced pressure, and then lyophilized from ACN:water (1:1) to give 138.8 mg; 0.120 mmol of N-[3-[2-[2-[3-[5-[(3aS,4S,6aR)-2-oxo-1,3,3a,4,6,6a-hexahydrothieno[3,4-d]imidazol-4-yl]pentanoylamino]propoxy]ethoxy]ethoxy]propyl]-N′-[2-[4-[(E)-4-[(2S)-2-[8-amino-1-[4-(2-pyridylcarbamoyl)phenyl]imidazo[1,5-a]pyrazin-3-yl]-1-piperidyl]-4-oxo-but-2-enyl]piperazin-1-yl]ethyl]pentanediamide (Formula (3); Ex-1) (34%). Data: LCMS Rt=3.58 min; m/z 1151.5 (M+H)⁺; m/z 1149.5 (M−H)⁻; HPLC Rt=4.75 min; ¹H NMR (400 MHz, CDCl₃, 300 K): δ=8.80 (1H, s), 8.46 (1H, d, J=8.5), 8.36 (1H, d, J=4.7), 8.11 (2H, d, J=8.0), 7.89 (2H, d, J=8.1), 7.83 (1H, t), 7.66 (1H, s), 7.14 (1H, t), 7.10 (1H, d, J=4.9), 7.00-6.88 (2H, m), 6.80 (1H, m), 6.60 (1H, m), 6.49 (1H, d, J=15.2), 6.38 (1H, bs), 6.02 (1H, s), 5.40-5.20 (3H, m), 4.54 (1H, t), 4.36 (1H, t), 3.83 (1H, d, J=12.3), 3.64-3.52 (13H, m) 3.38 (6H, m), 3.23 (4H, m), 2.96 (1H, m), 2.76 (2H, m), 2.64-2.41 (11H, m), 2.24 (7H, m), 1.94 (3H, m), 1.93-1.54 (19H, m), 1.44 (3H, m).

Preparation of 2-methoxy-4-(4,4,5,5-tetramethyl-1,3,2-dioxaborolan-2-yl)-N-[4-(trifluoromethyl)-2-pyridyl]benzamide. 4-bromo-2-methoxy-N-[4-(trifluoromethyl)-2-pyridyl]benzamide (17.0 g, 45.3 mmol), bis(pinacolata)diboron (13.7 g, 54.3 mmol) and potassium acetate (8.8 g, 90.6 mmol) was taken up in dioxane (170 mL) and the reaction mixture was degassed under nitrogen for 10 minutes. Then, PdCl₂(dppf)₂.DCM (1.7 g, 2.2 mmol) was added and the reaction mixture was heated at 100° C. for 16 hours. The reaction mixture was cooled, water (300 mL) was added to this mixture and extracted with ethyl acetate (200 mL). The organic part was dried over sodium sulfate, filtered and concentrated to give a residue which was further purified by column chromatography using silica gel (100-200 mesh) and 0-10% ethyl acetate in hexane to give 2-methoxy-4-(4,4,5,5-tetramethyl-1,3,2-dioxaborolan-2-yl)-N-[4-(trifluoromethyl)-2-pyridyl]benzamide (14.4 g, 77.0%) as an off white solid. HPLC (Method E) Rt: 5.99 min; ¹H NMR (400 MHz, DMSO-d₆, 300 K): 10.9 (1H, s), 8.64 (1H, d, J=4.8 Hz), 8.56 (1H, s), 7.83 (d, 1H, J=7.6 Hz), 7.54 (d, 1H, J=4.8 Hz), 7.41-7.37 (m, 2H), 3.98 (s, 3H) and 1.32 (s, 12H).

### Example 4. Evaluation of BTK Probes

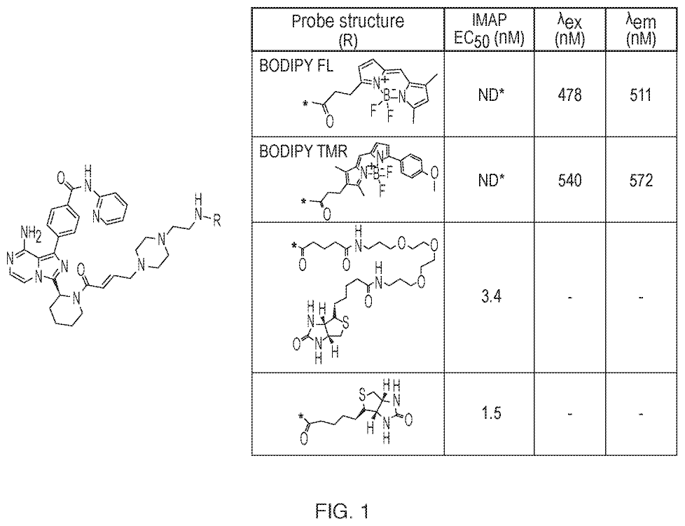

Several probes were synthesized and compared as part of the development of a target occupancy assay for BTK. The probes are structural analogues of acalabrutinib, with variation of linker length and the tag, as shown in FIG. 1. The probes bind covalently and irreversible to BTK. Both of the biotin-tagged probes were profiled in the BTK IMAP assay (described below) to investigate potency for BTK inhibition, and showed potent inhibition of BTK with an IC₅₀ of 3.4 for Formula (3) and 1.5 nM for Formula (4). Determining the potency for the probes with the fluorescent labels is not possible due to interference of fluorescence in the IMAP assay.

### Example 5. Procedure for Binding Probes to Recombinant BTK and Analysis of Target Occupancy on Gel and Western Blot

### Example 5.1. Procedure for Binding Probes to Recombinant BTK and Results of Analysis of Target Occupancy on Gel and Western Blot

To test different BTK target occupancy probes and lysis buffers, probes were incubated with recombinant BTK protein for 2 hours and subsequently run using sodium dodecyl sulfate polyacrylamide gel electrophoresis (SDS PAGE). When using the boron-dipyrromethene (BODIPY) tagged probes (such as Formula (5) and Formula (6)), the gel is measured directly using a fluorescence imager. BTK probe (final concentration 0.1 μM) is incubated with 125 ng BTK for 2 hours at room temperature. Afterwards, sample buffer is added and run on a SDS-PAGE gel. Fluorescent probes such as Formula (5) and Formula (6) are quantified in gel using the E tan Imager from GE Healthcare, suitable for the emission and detection of fluorescent signal for BODIPY tags.

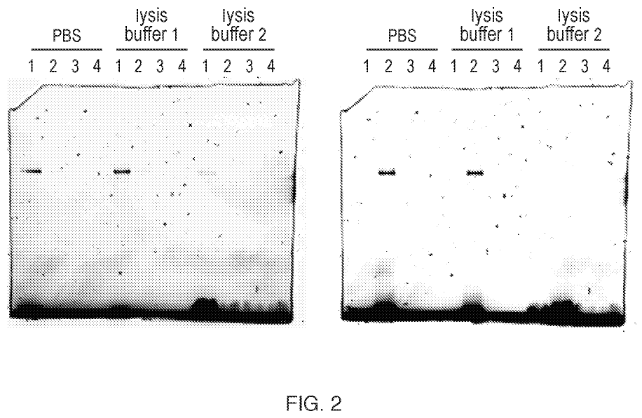

Both the fluorophore labeled target occupancy probe (Formula (5)) and the BODIPY-TMR labeled probe (Formula (6)) show clear binding when performing the incubation of the probes with recombinant BTK in either PBS or lysis buffer 1 (50 mM Tris-HCl pH 7.5, 250 mM sucrose, 5 mM MgCl₂, 1 mM DTT, 0.025% digitonin). When using lysis buffer 2 (50 mM Tris-HCl pH 7.4, 150 mM NaCl, 1 mM EDTA, 1% Triton X100), there is minimal or no detectable binding to BTK for Formula (5) and Formula (6), respectively, as shown in FIG. 2. Lysis buffer 1 thus showed improved performance in the assay.

For the biotin probes (such as Formula (3) and Formula (4)), samples are run using SDS-PAGE gel, followed by transferring the protein to a polyvinylidene fluoride (PVDF) membrane by Western blotting. Subsequently, the blot is blocked overnight at 4° C. in TBS-T (10 mM TRIS pH 7.4, 100 mM NaCl, 0.1% Tween 20)+2.5% (w/v) skimmed milk powder). The blot is washed 4× with TBS-T and then incubated with Streptavadin-HRP for 1 hour at room temperature. The blot is washed again 4× with TBS-T before adding the chemiluminescent substrate, followed by measurement of the chemiluminescence signal.

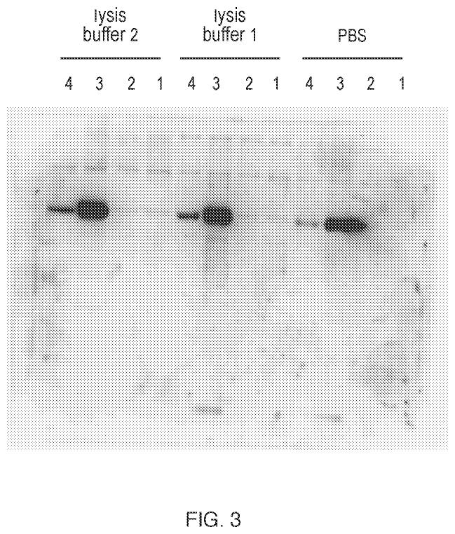

For the biotin labeled probes, as described above, the gel was transferred to a membrane by Western blotting and probed afterwards subsequently with Streptavadin-HRP. When using the biotin labeled probes, similar signals are observed for the individual probes in either PBS or the two different lysis buffers, as shown in FIG. 3. The BTK probe of Formula (3) clearly yielded a stronger signal compared to the BTK probe of Formula (4).

Overall, the results indicate that the probe of Formula (3) is surprisingly sensitive (in spite of its reduced potency as determined by the IMAP assay), and furthermore, lysis buffer 1 produces surprisingly superior results.

### Example 5.2. Detailed Procedure for Binding Probes to Recombinant BTK and Analysis of Target Occupancy on Gel and Western Blot

SDS-PAGE procedures are known to those of ordinary skill in the art. A non-limiting example of a SDS-PAGE procedure is as follows:
1. Add 1.1 μL of probe to 10 μL of each sample (recombinant BTK or cell lysate); final probe concentration 0.1 μM.
2. Incubate 2 hour at room temperature.
3. Add 4 μL sample buffer/DTT (200 μM) and incubate for 5 minutes at 95° C.
4. Vortex and keep on ice until loading on gel.
5. Spin down shortly and load 15 μL on a 12 slot Novex gradient gel (4-12% Bis-Tris gel).
6. Run in NuPage MOPS SDS running buffer.
7. After running the samples on gel, remove the gel from the surelock holder.
8. For the samples on gel with the fluorescent probe, the gels are measured directly using an imager suitable for measuring the fluorescent label on the probes (e.g., E tan Imager; GE Healthcare (Cy2: 480/30 excitation, 530/40 emission, Cy3: 544 excitation, 570 emission).
9. For the biotin labeled probes, the gel is blotted to a PVDF membrane, using the Western blot procedure below.

Western blot procedures are known to those of ordinary skill in the art. A non-limiting example of a Western blotting procedure is as follows:
1. After removing the gel from the surelock holder, place the gel on 2 layers of Whatman paper soaked in blot buffer (25 mM TRIS/192 mM Glycine in MQ/MeOH (20% MeOH (v/v)).
2. Place PVDF, 0.45 μm membrane (Imobilon, Sigma Aldrich, #P2563-10EA) on top of the gel and cover with 2 layers of Whatman paper soaked in blot buffer. Make sure there are no air bubbles between gel, blot, and paper.
3. Place gel, membrane, and Whatman paper, together with fibre-pads on both sides, into the holder for Western blotting and add to mini 2D-cell filled with blot buffer.
4. Run for 1 hour at 100 V.
5. Remove gel+blot from holder and transfer blot to a 50 mL tube and add 20 mL block buffer (TBS-T (50 mM TRIS pH 7.4, 150 mM NaCl, 0.1% Tween 20)+5% (w/v) skimmed milk powder). Incubate 1 hour at room temperature.
6. Wash the blot 4×5 minutes using TBS-T (TBS-T, 10 mM TRIS pH 7.4, 100 mM NaCl, 0.1% Tween 20).
7. Add 0.6 μg/mL Streptavadin-HRP (ELISA grade, Life Technologies, catalog no. #SNN2004) in 5 mL TBS-T in a 50 mL tube.
8. Incubate for 1 hour at room temperature.
9. Wash the blot 4×5 minutes using TBS-T.
10. Add 2 mL SuperSignal Western Pico Chemiluminescent Substrate (ThermoFisher Scientific, catalog number34077).
11. Measure the chemiluminescence signal using an imager (e.g., UVP AUTOCHEMI system with Hamamatsu 1394 C8484-51-03G camera)

### Example 6. Procedure for Assay of Target Occupancy and PLCγ2 Phosphorylation in Ramos B Cells

### Example 6.1. Procedure and Results for Assay of Target Occupancy and PLCγ2 Phosphorylation in Ramos B Cells

Ramos B cells are plated in 24-well culture plates at 1×10⁷ cells per well in a total volume of 1 mL. The cells are allowed to rest overnight at 5-7% CO₂ and 37° C. For BTK target occupancy, a 5 point 10× serial dilution from 100 μM to 0.1 μM in DMSO is prepared, leading to a final compound concentration range in the assay from 1 μM to 0.001 μM. Cells are harvested, washed and lysed in 200 μL cold (2-8° C.) lysis buffer (50 mM Tris-HCl pH 7.5, 250 mM Sucrose, 5 mM MgCl₂, 1 mM DTT, 0.025% digitonin) and quantified using the BTK target occupancy ELISA procedure described below.

For PLCγ2 phosphorylation, a 10 point 110 serial dilution of acalabrutinib from 0.316 mM to 10 nM is prepared, resulting in a final compound concentration range in the assay from 3.16 μM to 0.1 nM). Compound solutions are further diluted in assay medium with a final DMSO concentration of 1% in the cell assay. Ramos cells are plated in 24-well culture plates at 3.5×10⁶ cells per well in a total volume of 1 mL culture medium and allowed to rest for 1.5 hours in a humidified atmosphere at 5-7% CO₂ and 37° C., prior to adding the test compound. Cells are incubated for 2 hours with the test compound, before stimulation with 100 mM H₂O₂ for 10 min. Cells are placed on ice, transferred to Eppendorf tubes, spun down and washed once with 1 mL cold PBS. Afterwards, cells are lysed in 70 μL lysis buffer supplemented with 1 mM PMSF and Complete protease inhibitor cocktail. 7.5 μL of each sample is run on a 4-12% Bis-Tris gel followed by Western blotting. The blot is probed with the pPLCγ2 antibody (Y759, Cell Signaling, catalog no. 3874S) and anti-Rabbit IgG HRP (Promega, catalog no. W401B) is used for detection.

The use of the biotin-tagged probe of Formula (3) also allowed for the development of an ELISA-based assay to measure target occupancy in cells that have been exposed to acalabrutinib or other covalent BTK inhibitors. In the ELISA procedure, the cell lysates are incubated with Formula (3) prior to being added to a well of a 96-well ELISA plate that has been coated with anti-BTK. The BTK-probe complex present in the cell lysate will be captured by anti-BTK. Subsequently, the biotin tag on the probe is used for the binding of Streptavadin-HRP. Detection is done by using the turnover of a chemiluminescent substrate by the peroxidase.

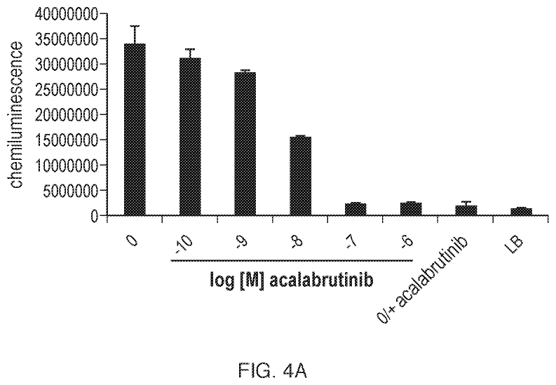

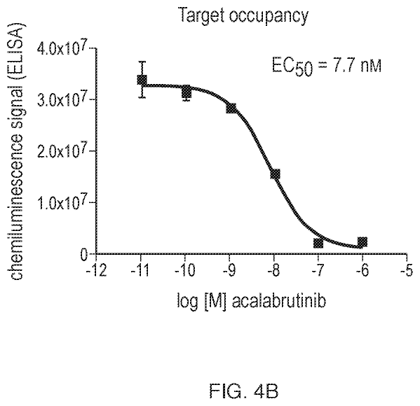

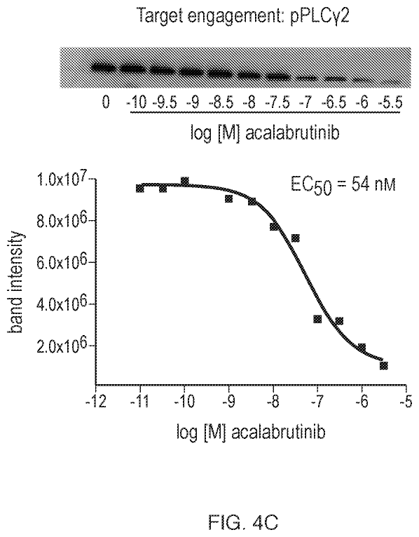

When incubating Ramos B cells with a dose range of acalabrutinib, followed by an incubation of the cell lysates with the target occupancy probe Formula (3), there is a decrease in the binding of the probe with an increase in the concentrations of acalabrutinib the cells were treated with (FIG. 4A). At 0.1 and 1 μM acalabrutinib, the remaining signal is identical to the background signal. The background signal is generated by the addition of a high concentration of acalabrutinib (1 μM) or when using lysis buffer (LB) only.

The results from the bar graph were also used to generate for a dose response curve and to calculate the EC₅₀ for the BTK target occupancy (FIG. 4B). To compare BTK target occupancy with a functional regulation of BTK activity, the phosphorylation of PLCγ2, a direct substrate of BTK, was investigated as a target engagement readout for BTK activity (FIG. 4C). With an EC₅₀ of 7.7 nM for target occupancy, and of 54 nM for target engagement, respectively, the result showed a correlation between BTK occupancy and activity. Differences in absolute numbers for the EC₅₀ may be explained by the technical procedure, but may also, in part, be due to the fact that the level of target occupancy may not translate directly to the same level of regulation of BTK activity. For example, in order to achieve 50% inhibition in kinase activity, it may require more than 50% of BTK being blocked by an inhibitor.

### Example 6.2. Detailed Procedure for Target Occupancy and PLCγ2 Phosphorylation in Ramos B Cells

The Ramos assay was developed as a cellular in vitro assay in the profiling and selection of inhibitors of B cell receptor (BCR) activation in B cells, investigating the effect of inhibition of BTK on anti-IgM-induced MIP1ß production. This cell line may also be used to investigate the effect of BTK inhibitor on the target occupancy and target engagement of BTK. The latter is being investigated by directly measuring the regulation (phosphorylation) of PLCγ2, a direct substrate of BTK. Other variations of this assay are known to those of skill in the art and may be used.

The cell line used is Ramos.2G6.4C10. The materials and reagents used are as follows:
1. DMEM F12 modified (GIBCO, catalog no. 041-94895 M or similar quality).
2. Sterile 96-well cell culture plates (Nunc, catalog no. 167008 or similar quality).
3. Penicillin/streptomycin, 10 kU Pen+10 mg/ml Strep (GibcoBRL, catalog no. 15140-122).
4. Fetal Bovine Serum (Hyclone, catalog no. SH30406.02 or similar quality), not heat inactivated.
5. Anti-PLCγ2 antibody (Cell Signaling, catalog no. 3874S).

Details on materials for used for assessing BTK target occupancy by ELISA are described in Example 6.3.

The equipment used is as follows:
1. Imager (e.g., UVP, AUTOCHEMI system with Hamamatsu 1394 C8484-51-03G camera)
2. Gel electrophoresis equipment (e.g., Life Technologies, XCell SURELOCK Mini-Cell, catalog no. EI0001)
3. Western blot equipment (e.g., BioRad, Min-Protean 3 Mini Trans-Blot Module, catalog no. 165-3317)

The following method may be used for the Ramos assay:
1. Thaw cryopreserved vial of Ramos cells
2. Culture the cells in culture flask in a humidified atmosphere at 5-7% CO₂, 37° C. on DMEM F12 modified supplemented with Penicillin/streptomycin (80 U/mL; 80 μg/mL) and 7.5% non-heat inactivated FBS.
3. Cells are cultured at 37° C., 5% CO₂ and transferred 3 times a week. Count a sample of the cell suspension and seed a culture flask with a cell seeding concentration of 2×10⁵ cells/mL (Monday), 2×10⁵ cells/mL (Wednesday), and 1.5×10⁵ cells/mL (Friday). Do not allow the cells to grow to a cell concentration of more than 1×10⁶ cells/ml.
4. For BTK target occupancy stock solutions (10 mM) of the test compounds in DMSO are prepared and stored at room temperature. Serial dilutions of compounds are made in 100% DMSO (e.g., for a 5 points 10× serial dilution from 100 μM to 0.1 μM leading to a final compound concentration range in the assay from 1 μM to 0.001 μM in assay medium.
5. The day before stimulation, plate the cells in 96-wells culture plates at 1×10⁵ cells per well in a total volume of 200 μL culture medium. Allow the cells to rest in a humidified atmosphere at 5-7% CO₂ and 37° C. overnight.
6. On the day of stimulation add 20 μL test compound and incubate for 2 hours in a humidified atmosphere at 5-7% CO₂ and 37° C. with a final DMSO concentration of 1% in the cell assay. This percentage of DMSO has no effect on the cells.
7. For BTK target occupancy, cells are harvested (1×10⁷ cells total), washed and cell pellet was lysed in 200 μL cold (2-8° C.) lysis buffer 1 (50 mM Tris-HCl pH 7.5, 250 mM sucrose, 5 mM MgCl₂, 1 mM DTT, 0.025% digitonin).
8. For further experimental details on BTK target occupancy ELISA measurements, see Example 6.3.
9. For PLCγ2 phosphorylation, stock solutions (10 mM) of the test compounds in DMSO are prepared and stored at room temperature. Serial dilutions of compounds are made in 100% DMSO (e.g. for a 10 points √10 serial dilution from 0.316 mM to 10 nM leading to a final compound concentration range in the assay from 3.16 μM to 0.1 nM). Compound solutions are further diluted in assay medium with a final DMSO concentration of 1% in the cell assay.
10. The day before stimulation, cells are plated in 24-well culture plates at 3.5×10⁶ cells per well in a total volume of 1 mL culture medium. Allow the cells to rest in a humidified atmosphere at 5-7% CO₂ and 37° C. for 1.5 hours.
11. Add 125 μL test compound and incubate the cells for 2 hours.
12. Stimulate the cells with 100 mM H₂O₂ for 10 min.
13. Place the cells on ice and transfer the cells to Eppendorf tubes.
14. Centrifuge at 5000 rpm for 5 minutes at 4° C.
15. Remove the supernatant and wash the cell pellet with 1 mL cold PBS, centrifuge at 5000 rpm for 5 minutes at 4° C.
16. Lyse the cells in 70 μL lysisbuffer (Life Technologies, catalog no. FNN0011) supplemented with 1 mM PMSF (Fluka, catalog no. 93482) and Complete protease inhibitor cocktail (Roche, catalog no. 11873580001).
17. Run 7.5 μL of each sample on a 4-12% Bis-Tris gel followed by Western blotting as described in Example 5.2.
18. Blots were blocked in TBS-T+5% (w/v) skimmed milk powder for 1 hour at room temperature. pPLCγ2 antibody was used 1:1000 and incubated overnight at 4° C. Anti-Rabbit IgG HRP detection antibody (Promega, catalog no. W401B) was used at a final concentration of 50 ng/mL and incubated for 1 hour at room temperature.

### Example 6.3. Detailed Procedure for ELISA BTK Target Occupancy

The following method may be used for the ELISA BTK target occupancy assay. Other variations of this assay are known to those of skill in the art and may also be used.
1. Coat a 96-well plate (Optiplate, Perkin Elmer, catalog no. 6005290) with 125 ng/well anti-BTK (BD Biosciences catalog no. 611117) in 100 μL PBS, overnight at 4° C.
2. Wash 2×200 μL/well PBS-Tween 0.05% (PBST).
3. Block with 245 μL/well PBST+3% bovine serum albumin (BSA), for 2 to 3 hours at room temperature while shaking.
4. During blocking, incubate samples with acalabrutinib and probe in Eppendorf tubes:
   a. Centrifuge cells 5 minutes at 5000 rpm and 4° C. in Eppendorf centrifuge.
   b. Remove supernatant, dilute BTK protein or resuspend cell pellet in cold (2-8° C.) lysis buffer (see below). For amount of cells/μL, see detailed protocols for Ramos (Example 6.2), canine B cells (Example 7.2) and human PBMCs (Example 8.2).
   c. Incubate on ice for 30 minutes while shaking. Vortex every 10 minutes.
   d. Centrifuge for 10 minutes at 14000 rpm and 4° C. in Eppendorf centrifuge.
   e. Transfer supernatant to new tube.
   f. For each sample; incubate 210 μL with 1 μM acalabrutinib (=+acalabrutinib) and 210 μL with no additive (=− acalabrutinib) on ice for 1 hour.
   g. Incubate all samples (+ and − acalabrutinib) with 0.1 μM biotin BTK probe (e.g., Formula (3)) on ice for 1 hour.
5. Wash wells 2×245 μL/well PBST.
6. Add (in duplicate) 100 μL of each sample (with and without Formula (3)) to the wells.
7. Incubate for 2 hours at room temperature while shaking.
8. Wash 4×PBST, 200 μL/well.
9. Add 100 μL/well PBST+1% BSA+0.1 μg/ml streptavidin-conjugated horseradish peroxidase (Strep-HRP, Invitrogen, catalog no. SNN2004).
10. Incubate for 1 hour at room temperature while shaking.
11. Wash with 3×PBST 200 μL/well, then with 2×PBS 200 μL/well.
12. Add 100 μL/well SuperSignal ELISA Pico Chemiluminescent Substrate (ThermoFisher Scientific, catalog no. 37070) or SuperSignal ELISA Femto Chemiluminescent Substrate (ThermoFisher Scientific, catalog no. 37075).
13. Measure luminescence on Envision 2102 Multilabel Reader equipped with an Ultra Sensitive Luminescence PMT detector or comparable equipment, 0.3 sec/well (suggested settings include: 96 w luminescence aperture).

Lysis buffer 1 contains the following components: 50 mM Tris-HCl pH 7.5, 250 mM sucrose, 5 mM MgCl₂, 1 mM dithiothreitol (DTT), and 0.025% digitonin.

Lysis buffer 2 contains the following components: 50 mM Tris-HCl pH 7.4, 150 mM NaCl, 1 mM ethylenediaminetetraacetic acid (EDTA), and 1% Triton X-100.

### Example 7. BTK Target Occupancy in Canine Peripheral B Cells

### Example 7.1. Procedure and Results for BTK Target Occupancy in Canine Peripheral B Cells

PBMCs are isolated from the blood of dogs by Ficoll Paque procedure. The CD21+ cells in the PBMCs were purified by MACS sorting. To this end, PBMCs are resuspended in 100 μL MACS buffer (PBS+0.5% BSA+2 mM EDTA) and 50 μL mouse anti-canine CD21− PE antibody (Abd Serotec catalog no. MCA1781PE) and incubated for 10 minutes in the dark at 4° C. After washing the cells by adding 10 mL of cold MACS buffer, the cell pellet is resuspended in 80 μL of MACS buffer per 10⁷ total cells. Add 20 μL of Anti-PE microbeads per 10⁷ total cells is added (usually 80-100 μL) and incubated for 15 minutes at 4° C. Following a wash with MACS buffer, up to 10⁸ cells are resuspended in 500 μL of MACS buffer. A MS column was placed in the magnetic field of the MiniMACS separator. The Apply cell suspension is applied onto the column and the unlabeled cells that pass through are rerun over the column. The pass through is the “CD21− Fraction.” The column is washed with MACS buffer before pipetting 1 mL of FACS buffer onto the column. Magnetically labeled cells are immediately flushed out by firmly pushing the plunger into the column. This fraction is the “CD21+ Fraction.” For more detail on the experimental procedure, see Example 7.2.

For the measurement of the BTK target occupancy, the CD21+ B cells are used in the BTK target occupancy ELISA with normalization for the number of B cells. When using the CD21− cell fraction, the cell number is normalized versus the B cell number used for the same dog. Cell pellet is lysed in 100 μL cold (2-8° C.) lysis buffer (50 mM Tris-HCl pH 7.5, 250 mM sucrose, 5 mM MgCl₂, 1 mM DTT, 0.025% digitonin). For the BTK target occupancy lysate from 4.10⁵ cells in 100 μL cold lysis buffer is used per well in the target occupancy ELISA as described in Example 6.3.

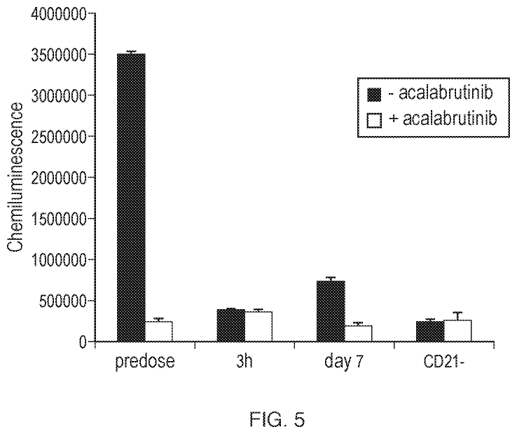

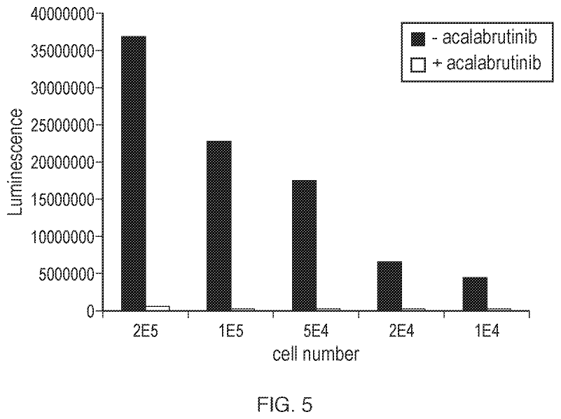

BTK target occupancy in canine peripheral B cells was investigated using CD21+ B cells from dogs with spontaneous development of lymphomas treated once daily with acalabrutinib (2.5 mg/kg, peroral). CD21+ B cells were isolated from blood draws at different time points: predose (t=0), 3 hours post dose, and on day 7 prior to receiving a repeat dose. In this setup, the BTK target occupancy reflects the in vivo occupancy of BTK after oral dosing of acalabrutinib. After isolation of the B cells, the cells were lysed (normalized for the same number of B cells in the different samples) and split in two equal portions for an incubation in presence or absence of exogenous acalabrutinib (1 μM) to determine background signal in the ELISA (FIG. 5). The blue bars represent the incubation in the absence of exogenous acalabrutinib and the red bars the incubation in the presence of exogenous acalabrutinib. The difference between the two bars represents the amount of free BTK present in the cell lysate. The predose samples represent the total signal that can be achieved within the individual dog. As shown in FIG. 5, at 3 hours post dosing of the drug, there is no difference in the signal between the samples incubated in the presence or absence of exogenous acalabrutinib. This illustrates that there is full BTK target occupancy in canine peripheral B cells, 3 hours after the dogs received a dose of 2.5 mg/kg acalabrutinib. On day 7, blood from dogs was drawn prior to the next dose of acalabrutinib, so 1 day after receiving the preceding dose of acalabrutinib. The bars of the samples treated with or without exogenous acalabrutinib show there is some free BTK available, but clearly not a full recovery of free BTK. When calculated as a percentage of the predose sample, 83% of BTK is still occupied with acalabrutinib. This indicates a slow return of free BTK in dogs as a consequence of de novo synthesis of the protein.

To assess BTK target occupancy in the CD21+ peripheral B cell population, the BTK probe of Formula (3) was incubated with the PBMC that were depleted for CD21+ B cells. This is referred to in FIG. 5 as the CD21− population. Lysate from the same amount of cells was used as the CD21+ cells in the other samples. The results show no difference between the cell lysates incubated in the presence or absence of high dose exogenous acalabrutinib. This confirms that the BTK target occupancy signal is selective for the CD21+ B cells.

### Example 7.2. Detailed Procedures for Isolation of PBMCs from Dog Blood, B Cell Purification, and BTK Target Occupancy

Isolation of PBMCs from dog blood may be performed using the following procedure. Other suitable procedures are known to those of ordinary skill in the art. Approximately 8-9 mL blood is drawn using sodium-heparin as anticoagulant and stored at room temperature until the PBMC preparation. The following procedure is performed:
1. Bring Ficoll to room temp. Warm FACsVerse cell counting method.
2. For each blood sample, pipet 15 mL Ficoll to an empty 50 mL Accuspin tube (Sigma).
3. Centrifuge 800×g for 30 seconds at room temperature. The Ficoll should now be in the chamber below the frit.
4. Dilute blood to 1:2 with PBS (˜24 mL final volume) and pour freely into the Accuspin tube.
5. Centrifuge at 400×g for 30 minutes at room temperature with brake off.
6. After centrifugation, carefully aspirate, with a Pasteur or plastic pipet, the upper layer to within 2-3 mm of the opaque interface containing the mononuclear cells. Discard upper layer.
7. Using a clean Pasteur pipette transfer the lymphocyte layer to a clean labeled 15 mL centrifuge tube. It is critical to remove all of the interface but a minimum amount of Ficoll-Paque PLUS. Removing excess Ficoll-Paque PLUS causes granulocyte contamination.
8. Add at least 3 volumes RPMI (Roswell Park Memorial Institute) medium to the lymphocytes.
9. Mix tube by gentle inversion several times
10. Centrifuge at 250×g for 10 minutes.
11. Aspirate the supernatant and discard.
12. Resuspend the cell pellet with RPMI so that the final volume=10 mL.
13. Mix several times by gentle inversion.
14. Remove 50 μL of cells and transfer to FACS tube containing 200 μL RPMI (5-fold dilution). Set aside for cell count.
15. Centrifuge cells at 1500 RPM for 6 minutes.
16. Aspirate the supernatant and discard.
17. Suspend the lymphocytes in 1 mL of MACS buffer (PBS 0.5% BSA+EDTA 2 mM).
18. Count cells that are in the FACS tubes using FacsVerse:
   a. Add and name tubes.
   b. Obtain sample volume acquired and place a gate on viable cell excluding debris and RBCs.
   c. Record cells/mL and # total cells.
19. Split samples for cryopreservation: Prepare 2 cryovials/sample containing 2 million cells each in 1 mL freezing media (90% FBS, 10% DMSO).
20. Replace volume removed with MACS buffer.
21. The rest of cells will go on to the B cell isolation procedure described below.
22. Prepare the Nalgene® Mr. Frosty® Cryo 1° C. Freezing Container.
   a. Remove high-density polyethylene tube holder and foam insert from polycarbonate unit. Do not discard foam insert.
   b. Add 100% isopropyl alcohol to the fill line on the Mr. Frosty container. Do not overfill.
   c. Carefully replace foam insert and tube holder.
23. Place tubes containing sample into holes in tube holder of the Nalgene® Mr. Frosty® Cryo 1° C. Freezing Container. Place the container on dry ice. Leave undisturbed overnight.
24. Transfer cryotubes to liquid nitrogen on the next day.

B cell purification and isolation may be performed using the following procedure, comprising the three steps of magnetic labeling, magnetic separation, and a post-purification check. Other suitable procedures are known to those of ordinary skill in the art. MACS buffer (4° C.) is prepared as 1×PBS+0.5% BSA+2 mM EDTA.

The magnetic labeling step of B cell purification and isolation is as follows:
1. Set centrifuge at 4° C.
2. If cells appear clumpy, transfer cells to a new 15 mL conical with a 30 μm filter (Miltenyi Pre-Separation Filter 130-041-407).
   a. Wash filter 3 times with 500 μL of MACS buffer.
3. Spin at 400×g for 5 minutes and aspirate.
4. Resuspend cells in 100 μL MACS buffer.
5. Add 50 μL Mouse anti-canine CD21−PE antibody (Abd Serotec# MCA1781PE).
6. Mix well and incubate for 10 minutes in the dark at 4° C.
7. Wash cells by adding 10 mL of cold MACS buffer and centrifuge at 400×g for 5 minutes and aspirate.
8. Repeat the wash.
9. Resuspend cell pellet in 80 μL of MACS buffer per 10⁷ total cells.
10. Add 20 μL of anti-PE microbeads per 10⁷ total cells (usually 80-100 μL).
11. Mix well and incubate for 15 minutes at 4° C.
12. Wash cells by adding 10 mL of MACS buffer. Centrifuge at 400×g for 5 minutes and aspirate.
13. Resuspend up to 10⁸ cells in 500 μL of MACS buffer

The magnetic separation step of B cell purification and isolation is as follows:
1. Place MS column in the magnetic field of the MiniMACS separator.
2. Rinse column with 500 μL FACS buffer.
3. Place 15 mL conical labeled “CD21− Fraction” under the column and apply cell suspension onto the column.
4. Collect unlabeled cells that pass through and rerun over column.
5. Wash column with 3×500 μL MACS buffer.
6. Remove column from the separator and place it on a new 15 mL conical labeled “CD21+ Fraction.”
7. Pipette 1 mL of FACS buffer onto the column. Immediately flush out magnetically labeled cells by firmly pushing the plunger into the column.

The post-purification check step of B cell purification and isolation is as follows:
1. Take 10 μL from each tube (Pre, Negative Elution, Positive Elution), add to 190 μL MACS buffer in FACStubes (20 fold dilution).
2. Obtain cell counts from the CD21+ and C21− gates in the pre-, negative and positive column fractions.

BTK target occupancy may then be assessed as follows:
1. Spin down the CD21+ B cells and use a sample for BTK target occupancy ELISA normalized for the number of B cells. When using CD21− fraction normalize for total number of cells.
2. Lyse cell pellet in 100 μL cold (2-8° C.) lysis buffer 1 (see Example 6.3 for details on the lysis buffer) per 4×10⁵ cells. For the BTK target occupancy, lysate from 4×10⁵ cells in 100 μL cold lysis buffer is used per well.
3. Measure amount of free BTK using the BTK target occupancy ELISA. For details on the experimental procedure BTK target occupancy by ELISA, see Example 6.3.

### Example 8. BTK Target Occupancy in Human Peripheral Blood Mononuclear Cells

### Example 8.1. Procedure and Results for BTK Target Occupancy in Human PBMCs

Peripheral blood mononuclear cells (PBMCs) were isolated from Li-heparin blood samples by gradient density centrifugation using Ficoll Paque Plus™. These PBMCs were either used directly in the BTK target occupancy ELISA or cells were plated at 4.10⁵ cells per well in a total volume of 900 μL DMEM/F12 modified+10% FBS (Penn/Strep) in a flat bottom 24-well culture plate for incubation with acalabrutinib. Cell culture plates are placed at 37° C., 5% CO₂ for 1 hour to rest the PBMCs. Afterwards, a serial dilution of acalabrutinib is added and incubated with the cells for 2 hours. Cells are harvested and cell pellet lysed in 80 μL cold (2-8° C.) lysis buffer (50 mM Tris-HCl pH 7.5, 250 mM Sucrose, 5 mM MgCl2, 1 mM DTT, 0.025% digitonin). For the BTK target occupancy ELISA, lysate from 3.105 cells is used in a total volume of 100 μL cold lysis buffer per well.

For the PLCγ2 phosphorylation, 90 μL PBMCs were plated at 100,000 cells/well in RPMI+10% FBS (Penn/Strep) in a round bottom 96-deep well plate. Plate is placed at 37° C., 5% CO2 for 1 hour to allow the PBMCs to rest. Afterwards a serial dilution of acalabrutinib is added and incubated with the cells for 2 hours at 37° C., 5% CO₂. Subsequently, PBMCs are stimulated for 10 minutes at 37° C. with anti-IgM [10 μg/mL]+H₂O₂[3.3 mM] or not stimulated. Following the 10 minutes stimulation, 100 μL of 3.2% paraformaldehyde (1.6% final concentration) is added and left with the PBMCs for 10 minutes at 37° C. The plate is centrifuged for 5 minutes at 2000 rpm and the supernatant aspirated. 600 μL/well of 100% ice cold methanol is added and the plate is placed on ice for 30 minutes to permeabilize the cells. Cells are washed twice with PBS/0.5% BSA leaving 75 μL/well of liquid behind after aspiration and stained overnight at 4° C. with 25 μL of p-PLCγ2 (Y1217) unlabeled (Cell Signaling) (25 μL of cocktail to 75 μL of cells). Plate is washed twice with PBS/0.5% BSA (1 mL/well) and goat anti-rabbit secondary-Alexa 647 antibody (1:1000 dilution; Invitrogen) is added and plate is left for 30 minutes at 4° C. Afterwards, the plate is washed again twice with PBS/0.5% BSA (1 mL/well) and final volume adjusted to 100 μL with PBS/0.5% BSA. The samples are then analyzed on the FacsVerse cytometer. For further experimental detail, see Example 8.2.

In order to measure BTK target occupancy in future clinical studies, it is highly desirable to use PBMCs from patients without having to purify the B cells. Following an initial test using a high number of human PBMCs from healthy volunteers, a titration range of the cells was tested (FIG. 6). Again the cell lysates were incubated in the presence or absence of exogenous acalabrutinib, to correct for background signal in the BTK target occupancy ELISA. A good signal to noise ratio of the BTK probe of Formula (3) binding to free BTK versus the background was observed already with 1×10⁵ cells.

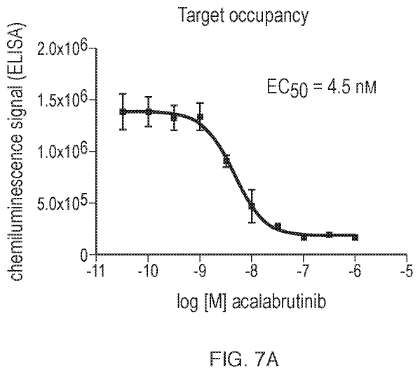

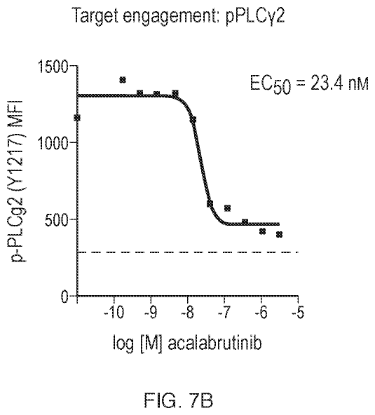

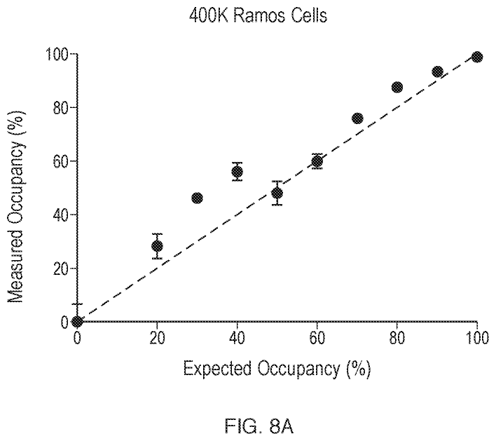

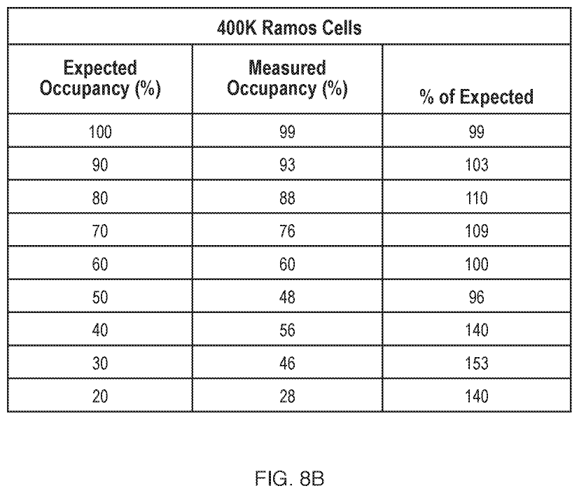

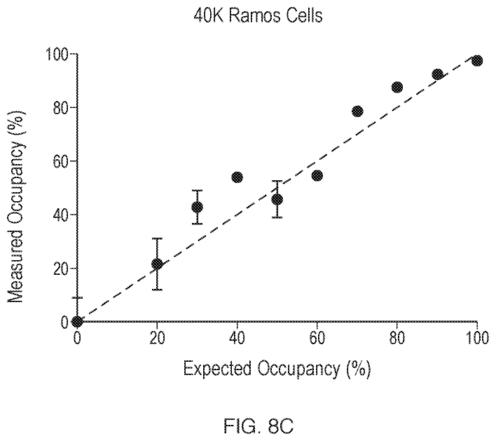

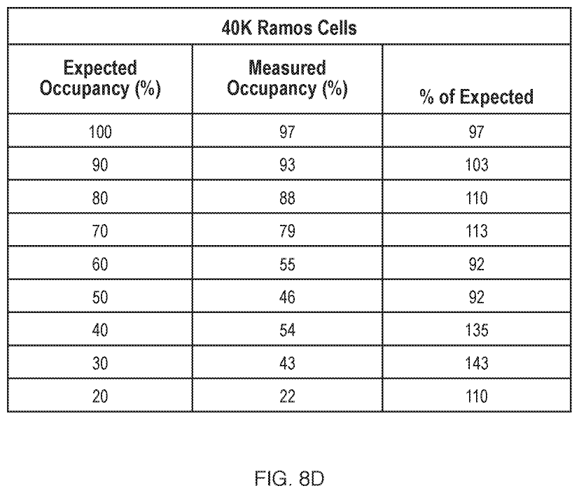

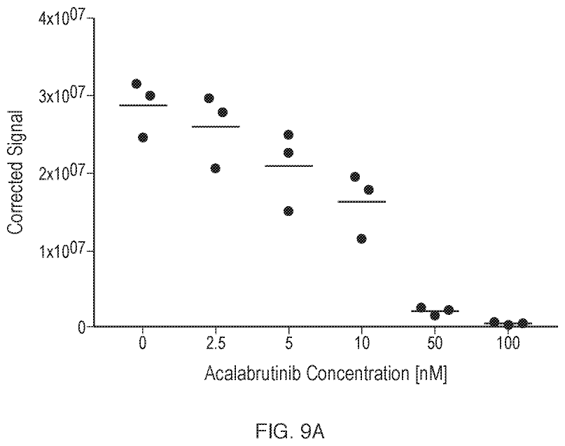

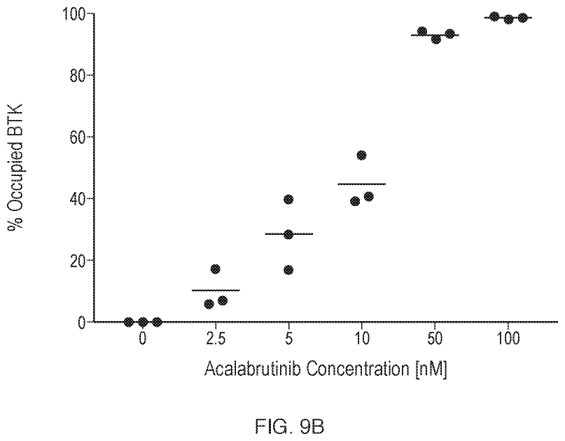

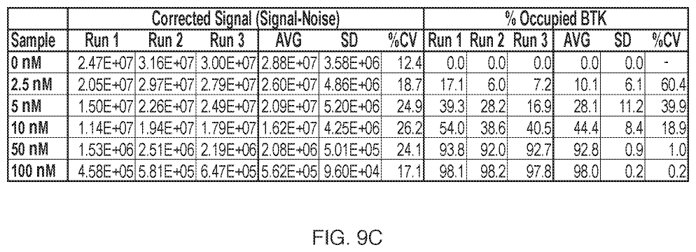

Similar to the Ramos B cells, human PBMCs were incubated with a concentration range of acalabrutinib. Following 2 hours of incubation with acalabrutinib, the cells were spun down, lysed and incubated with the BTK target occupancy probe of Formula (3). Results of the BTK target occupancy ELISA are summarized in FIG. 7. In order to get a good signal in the ELISA, 3×10⁵ PBMCs were needed. This is most likely due to loss of viable cells on cryopreservation and getting the cells in culture again. The EC₅₀ value for the target occupancy was 4.5 nM and was similar to that identified in the Ramos B cells. For the human PBMCs, the data on target occupancy were compared to activity of BTK in the peripheral B cells by, investigating the phosphorylation of PLCγ2 as a target engagement readout for BTK. Similar to the target occupancy assay, the PBMCs were preincubated for 2 hours in the presence of acalabrutinib, prior to stimulation with anti-IgM/H₂O₂ to induce the phosphorylation of PLCγ2. The EC₅₀ for acalabrutinib on the anti-IgM/H₂O₂ induced phosphorylation of PLCγ2 was 23.4 nM. In line with the data in the Ramos cells, the dose response for acalabrutinib for the target occupancy shows a good correlation with the target engagement readout.

### Example 8.2. Detailed Procedures for PBMC Collection from Li-Heparin Blood Samples, BTK Target Occupancy, and PLCγ2 Phosphorylation

PBMC collection from Li-heparin blood samples may be performed using the following procedures. Other PBMC collection procedures are known to those of ordinary skill in the art and may also be used. On the day of sampling, peripheral blood mononuclear cells (PBMC) are isolated from Li-heparin blood samples by gradient density centrifugation using Ficoll Paque PLUS (G.E. Healthcare Biosciences AB, Uppsala, Sweden). The following procedure may then be used:
1. Add 6 mL RPMI-culture medium to a 6 mL blood sample.
2. Overlay the 12 mL diluted blood sample to 9 mL Ficoll Paque Plus™ (G.E. Healthcare Biosciences AB, Uppsala, Sweden).
3. Centrifuge at 300×g for 30 minutes at room temperature.
4. Isolate the buffy coat. The isolated cells will be placed into 4 mL RPMI-medium and after completion of the isolation add up to 30 mL with RPMI-medium.
5. Centrifuge the cells for 5 minutes at 400×g at room temperature.
6. Remove the supernatant, resuspend in 1 mL RPMI-medium and add up to 12 mL with RPMI-medium.
7. Centrifuge the cells for 5 minutes at 400×g at room temperature.
8. Remove the supernatant, resuspend the pellet in 1 mL sterile 90% FBS+10% DMSO (freezing media, may be prepared in advance).
9. Transfer the resuspended cells into a pre-cooled polypropylene tube (Greiner Bio-One GmbH, Frickenhausen, Germany) and store on ice.
10. Prepare the Nalgene® Mr. Frosty® Cryo 1° C. Freezing Container (may be done prior to steps 1 to 7):
   a. Remove high-density polyethylene tube holder and foam insert from polycarbonate unit. Do not discard foam insert.
   b. Add 100% isopropyl alcohol to the fill line on the Mr. Frosty container. Do not overfill.
   c. Carefully replace foam insert and tube holder.
11. Place tubes containing sample into holes in tube holder of the Nalgene® Mr. Frosty® Cryo 1° C. Freezing Container. Place the container in bottom of ≤−75° C. freezer. Leave undisturbed overnight.
12. Remove the frozen tubes from the container and place the samples in a liquid nitrogen cryo storage system until use.

BTK target occupancy may then be assessed as follows:
1. Thaw a cryopreserved vial of PBMC and wash or use freshly prepared cells.
2. Plate 4×10⁵ cells per well in a total volume of 900 μL DMEM/F12 modified+10% FBS (Penn/Strep) per well to a flat bottom 24-well culture plate.
3. Place at 37° C., 5% CO₂ for 1 hour to rest.
4. Serial dilutions of compounds are made in 100% DMSO (e.g., for a 10 points √10 serial dilution from 0.1 mM to 3.16 nM leading to a final compound concentration range in the assay from 0.1 μM to 0.00316 nM). Compound solutions are further diluted in assay medium with a final DMSO concentration of 1% in the cell assay. This percentage of DMSO has no effect on the cells.
5. On the day of stimulation, add 200 μL test compound and incubate for 2 hours.
6. Cells are harvested and cell pellet lysed in 80 μL cold (2-8° C.) lysis buffer 1 (see Example 6.3 for details on the lysis buffer). For the BTK target occupancy lysate from 3×10⁵ cells in 100 μL cold lysis buffer is used per well.
7. For further experimentals on BTK target occupancy ELISA see Example 6.3.

PLCγ2 phosphorylation may be assessed as follows:
1. Add 90 μL cells at 100,000 cells/well in RPMI+10% FBS (Penn/Strep) to each plate (round bottom 96-deep well plate (Nunc)).
2. Place at 37° C., 5% CO₂ for 1 hour to rest.
3. Prepare 1000× compound dilution in 100% DMSO in a round or v-bottom 96-well plate. Make sure the last 2 wells (column 11 and 12) of the dilution series contain only DMSO with no compound. Final DMSO concentration will be 0.1% on cells.
4. Dilute 1000× compound series to 10× using with RPMI+10% FBS as the diluent.
5. Add 10 ul of 10× compound to wells.
6. Mix up and down and gently tap to swirl.
7. Incubate plate for 2 hour at 37° C., 5% CO₂.
8. Prepare a 10× stock [100 μg/mL] of goat F(ab′)2 anti-IgM (Southern Biotech) in RPMI+10% FBS.
9. Prepare a 10× stock [33.3 mM] of H₂O₂ from a 30% solution (Sigma).
10. Add 11 μL of 10× anti-IgM then immediately add 11 μL of H₂O₂ stock. Final concentration in solution anti-IgM [10 μg/mL]+H₂O₂[3.3 mM].
11. Stimulate for 10 minutes at 37° C. (float the plate in a water bath set to 37° C.).
12. For the unstimulated control add 11 μL of RPMI+10% FBS only.
13. Add 100 μL of 3.2% paraformaldehyde (1.6% final concentration) and mix for 10 minutes at 37° C.
14. Centrifuge for 5 minutes at 2000 RPM.
15. Aspirate supernatant.
16. Add 600 μL/well of 100% ice cold methanol.
17. Mix by vortexing.
18. Place plates on ice for 30 minutes to permeabilize the cells.
19. Wash twice with PBS 0.5% BSA, leaving 75 μL/well of liquid behind after aspiration.
20. Stain overnight with 25 μL of p-PLCγ2 (Y1217) unlabeled (Cell Signaling) (25 μL of cocktail to 75 μL of cells).
21. Mix and incubate overnight at 4° C.
22. Wash plates two times in PBS/BSA 0.5% (1 mL/well).
23. Add goat anti-rabbit secondary-Alexa 647 antibody (1:1000 dilution) (Invitrogen) for 30 minutes at 4° C.
24. Wash plates two times in PBS/BSA 0.5% (1 mL/well).
25. Bring volume up to 100 μL of PBS/BSA 0.5%.
26. Transfer cells to standard round bottom plate for flow cytometry.
27. Run plate on FacsVerse cytometer, gate data in FCSExpress and export median fluorescence values (MFI) to Excel. Make sure data is obtained from the non-apoptotic (cleaved PARP negative) and CD20+ gated B cell population.
28. Plot curves in GraphPad Prism for EC₅₀ determination.

### Example 9. BTK Immobilized Metal Ion Affinity-Based Fluorescence Polarization (IMAP) Assay

### Example 9.1. Summary of BTK IMAP Assay Procedure

In summary, BTK enzyme activity is measured using the IMAP (immobilized metal ion affinity-based fluorescence polarization) assay as outlined below. BTK enzyme (His-Btk (Millipore catalog no. 14-552), is diluted to 0.4 U/mL in Krebs Ringer (KR) buffer (10 mM Tris-HCl, 10 mM MgCl₂, 0.01% Tween-20, 0.05% NaN₃, 1 mM DL-dithiothreitol (DTT), 2 mM MnCl₂, pH 7.2). Serial dilution log 10 from 2 mM to 63.2 nM of test compounds are made in 100% DMSO. The dilutions in DMSO are then diluted 50-fold in KR buffer. Final compound concentration range in the assay is from 10 μM to 0.316 nM. Five μL/well of test compound in KR buffer (final DMSO concentration in the assay is 1%) is mixed with 5 μL/well of 0.4 U/mL BTK enzyme (final concentration in the assay is 0.1 U/mL). Test compounds and BTK enzyme are pre-incubated 60 minutes at room temperature, before adding 5 μL/well of 200 nM Fluorescin labeled substrate peptide (Blk/Lyntide substrate, #R7233, Molecular Devices) in KR buffer. Final peptide substrate concentration in assay is 50 nM. The kinase assay is started by adding 5 μL/well of 20 μM adenosine triphosphate (ATP) in KR buffer (final ATP concentration is 5 μM ATP, Km ATP in BTK IMAP assay). After incubation for 2 hours at room temperature, the enzyme reaction is stopped by adding 40 μL/well IMAP Progressive Binding Solution (according to suppliers (Molecular Devices) protocol using 75% 1× buffer A and 25% lx buffer B with 1:600 Progressive Binding Solution). After 60 minutes of incubation at room temperature in the dark, the FP signal is read. Fluorescence at 535 nm is measured using parallel and perpendicular filters to determine differences in rotation due to binding of the phosphorylated substrate peptide to the beads. Values are calculated as a percentage of the difference in readout (ΔmPi) of the controls with and without ATP.

### Example 9.2. Detailed BTK IMAP Assay Procedure

Inhibition of the activity of the protein kinase BTK can be measured with the assay described in this protocol. BTK is a cytoplasmatic non-receptor tyrosine kinase of the Tec family and is expressed in most hematopoietic tissues. BTK is critical for B cell development and function. The method is based on IMAP, which is a homogeneous fluorescence polarization (FP) assay based on affinity capture of phosphorylated peptide substrates. IMAP uses fluorescein-labeled peptide substrates that, upon phosphorylation by a protein kinase, bind to so-called IMAP nanoparticles, which are derivatized with trivalent metal complexes. Such binding causes a change in the rate of the molecular motion of the peptide, and results in an increase in the FP value observed for the fluorescein label attached to the substrate peptide. The IMAP assay is described in more detail in Sportsman, et al., Assay Drug Dev. Tech. 2004, 2, 205-214.

The following materials and reagents are used. These reagents are exemplary, and other suitable reagents are known to one of ordinary skill in the art.
Black 384-wells plates (for example #3575, Corning costar)
Dimethyl sulfoxide (DMSO), >99.0% (for example #41650, Fluka)
Adenosine 5′-triphosphate (ATP), 100% (absorbance), (for example #10 519987 001, Roche)
DL-Dithiothreitol (DTT), >99% (for example #D9163, Sigma)
Tris(hydroxymethyl)-aminomethane, >99.8% (for example #1.08382, Merck)
Magnesium chloride (MgCl₂), >99% (for example #1.05833. Merck)
Manganese (II) chloride tetrahydrate (MnCl₂), (for example #M5005, Sigma)
Polyoxyethylenesorbitan monolaurate (Tween-20), (for example #1379, Sigma)
Sodium azide (NaN₃), >99.5%, (for example #S2002, Sigma)
BTK, active enzyme (for example #14-552, Upstate)
IMAP buffer kit with Progressive Binding System (for example #R8127, Molecular Devices)
Fluorescein labeled Blk/Lyntide substrate (5FAM-EFPIYDFLPAKKK-NH2) (Molecular Devices #R7233)
Reader suitable for reading FP signal: Envision 2102 Multilabel Reader or comparable equipment (suggested settings include: dichroic mirror D505FP/D535, exitation filter: 480 nm center wavelength. Parallel and perpendicular filters: 535 nm center wavelength).

The following stock solutions are used. These stock solutions are exemplary, and other suitable reagents are known to one of ordinary skill in the art.
20 mM ATP dissolved in water and stored at −20° C.
1 M DTT dissolved in water and stored at −20° C.
1 M MnCl₂ dissolved in water
Reaction buffer: 10 mM Tris-HCl, 10 mM MgCl₂, 0.01% Tween-20, 0.05% NaN₃ pH 7.2
20 μM FI-peptide substrate in KR buffer (with only 1 mM DTT)
   Fresh KR buffer may be prepared just before use as follows: 50 mL reaction buffer+50 μL 1 M DTT (1 mM final conc.)+100 μL 1 M MnCl₂ (2 mM final conc.).
   Thaw enzyme on ice and keep the enzyme stock on ice during the assay. Quickly freeze the enzyme in dry ice/ethanol and store at −80° C. after use.
   Serial dilutions of test compounds are made in 100% DMSO (dilution plate). For example, a 10 point √10 serial dilution from 1 mM to 31.6 nM may be made. The solutions are diluted in assay buffer by a factor of 25 (in an intermediate plate). From the intermediate plate, 5 μL is transferred to the assay plate leading to a final compound concentration range in the assay from 10 μM to 0.316 nM.

The follows steps are performed:

Add 5 μL/well test compound in KR buffer (this solution contains 4% DMSO) or (in minimum, maximum and background wells) 5 μL/well KR buffer containing 4% DMSO (The final DMSO concentration in the assay is 1%);

Add 5 μL/well 0.4 U/ml (400 mU/mL) BTK enzyme diluted in ice-cold KR buffer to all wells (final BTK enzyme concentration in the assay is 0.1 U/mL (100 mU/ml));

Pre-incubate 60 minutes at room temperature in the dark;

Add 5 μL/well 200 nM F1-peptide substrate (100× dilution of the 20 μM stock in KR buffer, final F1-peptide substrate concentration in the assay is 50 nM) to maximum, minimum and compound wells and 5 L/well KR buffer to background wells;

Add 5 μL/well 20 μM ATP to compound, minimum and background wells (1000× dilution of the 20 mM stock in KR buffer, final ATP concentration in the assay is 5 μM) or 5 L/well KR buffer to maximum wells;

Incubate 120 minutes at room temperature in the dark;

Add 40 μL/well IMAP Progressive Binding Solution (IMAP Progressive Binding Solution: 75% 1× buffer A and 25% 1× buffer B with 1:600 diluted Progressive Binding Reagent, all kit contents) to all wells;

Incubate 60 minutes at room temperature in the dark; and

Read the FP signal.

On every 384 assay plate, 18 wells are used as minimum wells (wells with ATP, 0% effect), 18 wells are used as maximum wells (wells without ATP, 100% effect). 16 wells are used for measuring the background signal (everything but substrate). The difference between the maximum and minimum wells should be at least 50 mP (=window).

Evaluation of responses is performed for EC₅₀ generating assays. Both readings of the FP signal are first processed as follows:

$$mP = \frac{( ( {Count}_{para} - {BG}_{para} ) - ( G \times ( {Count}_{perp} - {BG}_{perp} ) ) )}{( ( {Count}_{para} - {BG}_{para} ) + ( G \times ( {Count}_{perp} - {BG}_{perp} ) ) )} \times 1000$$

where:
Countₚₐᵣₐ=measured parallel
Countₚₑᵣₚ=measured perpendicular
G=the grating factor that corrects for instrument bias which may be contributed by excitation and emission filters, beamsplitters, and polarizers
BGₚₐᵣₐ=background measured parallel
BGperp=background measured perpendicular

For each individual plate the following calculations are performed:
MAX Mean of the of the MAX wells which represent the 100% effect
MIN Mean of the mP's of the MIN wells which represent the 0% effect
Z prime

$$1 - ( \frac{3 \times ( STD\_MAX ) + 3 \times ( STD\_MIN )}{absolute ( MAX - MIN )} )$$

STD_MAX Standard deviation of the mP's of the MAX wells
STD_MIN Standard deviation of the mP's of the MIN wells
S/B ratio Signal/Background ratio (If MAX wells are the maximal signal then S/B is MAX divided by MIN; if MIN wells are the maximal signal then S/B is MIN divided by MAX)
Signal diff Difference between mean MAX and mean MIN
Effect Effect (%) is calculated for each well by correlating the mP with the mean of the mP's of the MIN wells and with the mean of the mP's of the MAX wells obtained from the same plate with the following formula:

$$\% Effect = \frac{( mP - MIN )}{( MAX - MIN )} \times 100 \%$$

The following parameters are calculated for all test compounds and for the reference compound across all replicates and stored in a computer database:
Effect Mean of the individual effects (%) for each compound concentration
Std Standard deviation of the individual effects for each compound (only calculated when there are 3 or more replicates)
Nbr Number of replicates included in the calculation of the mean effect

The individual effects at each concentration are used to fit a curve with the following four-parameter model:
x-axis: concentration (M)
y-axis: % effect

$$y = A + \frac{( B - A )}{( 1 + {( \frac{{10}^{C}}{x} )}^{D} )}$$

A=min
B=max
C=inflection point (log 10 (EC50)=−pEC50)
D=hill

All parameters are prefitted and are not locked. EC₅₀ is determined as the concentration (mol/L) at point of inflection.

### Example 10. Accuracy of BTK Target Occupancy Assay in Ramos Cells

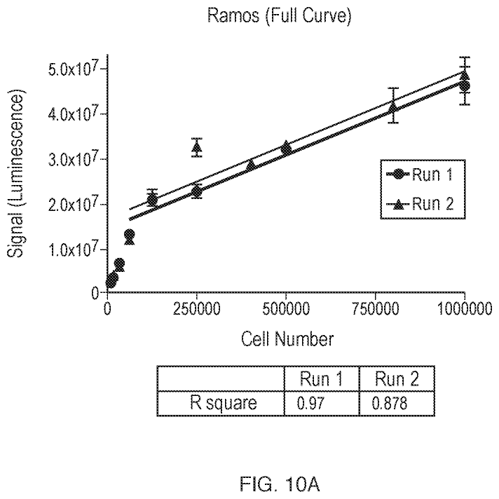

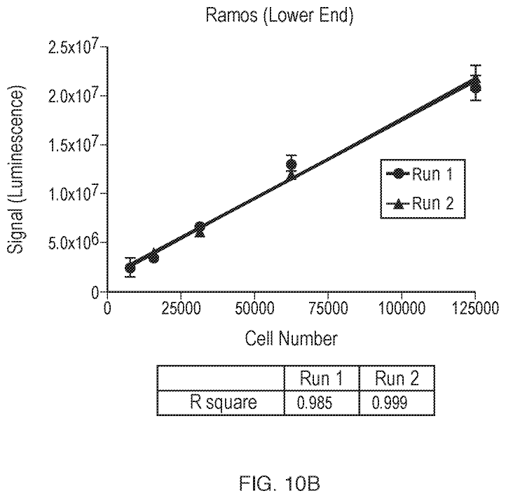

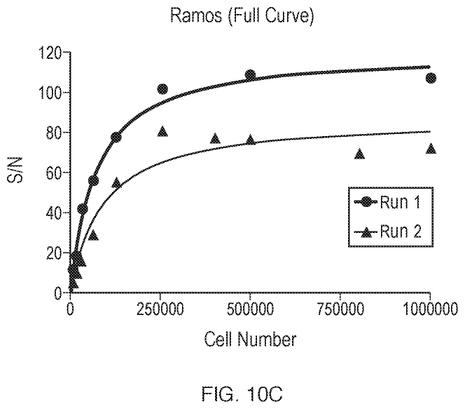

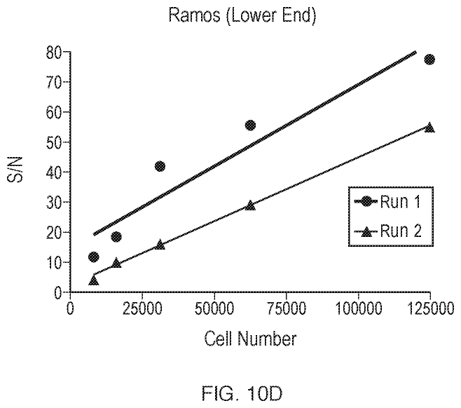

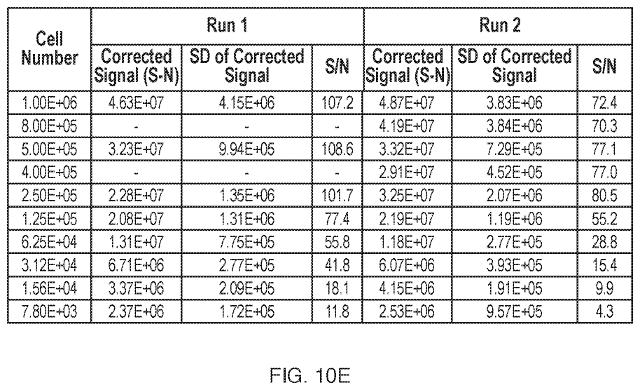

Ramos cells were treated with either 100 nM acalabrutinib (fully occupied BTK) or DMSO control (unoccupied BTK) in culture before being harvested and made into cell pellets. Cell pellets were lysed and the occupied and unoccupied lysates were mixed together in different ratios to generate a calibration curve. Curves with an equivalent of 400K Ramos cells (FIG. 10A and FIG. 10B), as well as 40K Ramos cells (FIG. 10C and FIG. 10D), were generated by diluting the occupied and unoccupied lysates tenfold before mixing them in different ratios.

The accuracy of the assay was highest at ≥80% occupancy, with the % expected value between 97% and 110%. At medium occupancy (50-79%), the % expected value was between 92% and 113%. At occupancy levels below 50%, the % expected value was between 110% and 153%, which is outside the accepted range of 80-120%. These data show that the assay has good accuracy at occupancy values ≥50%. Diluting the lysate used for the assay tenfold does not have a significant effect on assay accuracy.

As it stands, the assay is designed to measure the percent of unoccupied BTK in lysates compared with predose samples, and is not intended to provide absolute quantification of BTK protein within a particular sample.

### Example 11. Ramos Precision Experiment (Inter-Day)

Ramos cells were treated with acalabrutinib at varying concentrations to achieve a range of BTK target occupancies. Replicate cell pellets were thawed and tested in the BTK TO assay on 3 separate days. The corrected signal and % BTK occupancy were calculated to generate precision statistics (SD and % CV) for the inter-day precision at low and high occupancy levels.

To evaluate the inter-day precision of the BTK TO assay at different levels of BTK occupancy, Ramos cells were treated with varying concentrations of acalabrutinib (0 nM, 2.5 nM, 5 nM, 10 nM, 50 nM, and 100 nM), made into cell pellets, and cryopreserved at −80° C. The BTK TO assay was performed on the samples on three separate days. Luminescence signal corrected for background for three runs is shown in FIG. 7A, with % occupied BTK in FIG. 7B, and a summary of the precision values in FIG. 7C.

Overall, the corrected luminescence signals were more variable than the % BTK occupancy values. Coefficient of variation ranged from 12% and 27% across the dose range. Using the normalized BTK occupancy, calculated as percentage of the luminescence signal from Ramos cells not treated with acalabrutinib, higher precision was observed in samples with >40% occupied BTK.

The precision following repeated evaluation of BTK occupancy in Ramos cell lysates showed highest precision with the two samples having high BTK occupancy levels (1.0% CV at 92.8% occupancy and 0.2% at 98% occupancy). Lower precision estimates were obtained from samples with low BTK occupancy (60.4% CV at 10.1% occupancy, 39.9% CV at 28.1% occupancy, and 18.9% CV at 44.4% occupancy).

### Example 12. Linearity of Dilution

Ramos lysates were diluted from a top concentration representing 1 million cells per well to a final concentration representing 7.8 thousand cells per well and tested on two separate days to determine the linear range of the assay.

To determine dilution linearity, the luminescence signal corrected for background was compared in assays with an extended range of BTK lysate from 1×10⁶ cells to 7.8×10³ Ramos cells (FIG. 10A). While the corrected signal does appear to increase in a linear fashion between 125,000 and 1,000,000 cells for the two runs (R²=0.97 and 0.878), the true linear range is likely between 7800 and 125,000 cells (FIG. 10B), with R²=0.985 and 0.999.

Signal-to-noise ratio starts to plateau at cell counts over 250K (FIG. 10C). Thus, the Ramos QC control of 400K cells represents a high signal-to-noise ratio and the Ramos QC control of 40K cells represents a signal-to-noise ratio close to the expected luminescence range for a patient sample, within the linear range of the luminescence curve. As can be seen in FIGS. 10C-E, the S/N ratio shows high variability between runs, likely due to small day-to-day assay changes in absolute background and luminescence values, which are magnified when signal is divided by background. The corrected luminescence signal shows less variation in this particular experiment. Therefore, a ratio between the 400K and 40K Ramos controls may be a better indicator to estimate the working range of the assay than S/N or corrected luminescence signal.

1. A method for determining a drug target occupancy of Bruton's tyrosine kinase (BTK) in a patient after treatment of the patient with a BTK inhibitor, comprising the steps of:
(a) obtaining a tissue sample from the patient;
(b) separating a population of cells from the tissue sample;
(c) contacting a BTK probe with the population of cells;
(d) detecting the amount of BTK bound to the BTK probe using an assay;
(e) determining the drug target occupancy of BTK in the population of cells based on the amount of BTK bound to the BTK probe; and
(f) optionally performing a second assay for PLCγ2 phosphorylation;
wherein the BTK probe is a compound according to: 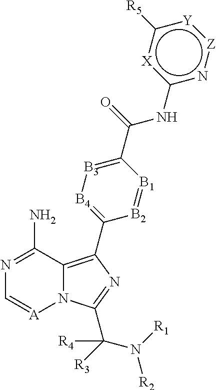
or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
L₁ is selected from the group consisting of heterocycloalkyl and heteroalkyl;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

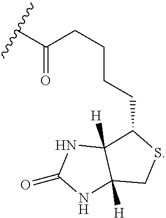 [SMILES: *C(=O)CCCC[C@@H]1SC[C@@H]2NC(=O)N[C@@H]21]

2. The method of claim 1, wherein L₁ is selected from the group consisting of: 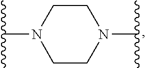 [SMILES: *N1CCN(*)CC1]
—O—, —(C₁₋₅)alkoxy-, and —[(C₁₋₁₀)alkyl]amino-.

3. The method claim 1, wherein R₁₂ is:

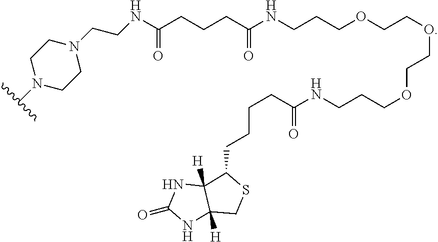 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

4. The method of claim 1, wherein the assay is an enzyme-linked immunosorbent assay (ELISA).

5. The method of claim 1, wherein the tissue sample is selected from the group consisting of blood, lymphatic tissue, and tumor biopsy tissue.

6. The method of claim 5, wherein the tissue sample is blood, and wherein the population of cells are peripheral blood mononuclear cells.

7. The method of claim 1, wherein the BTK probe is a compound selected from the group consisting of: 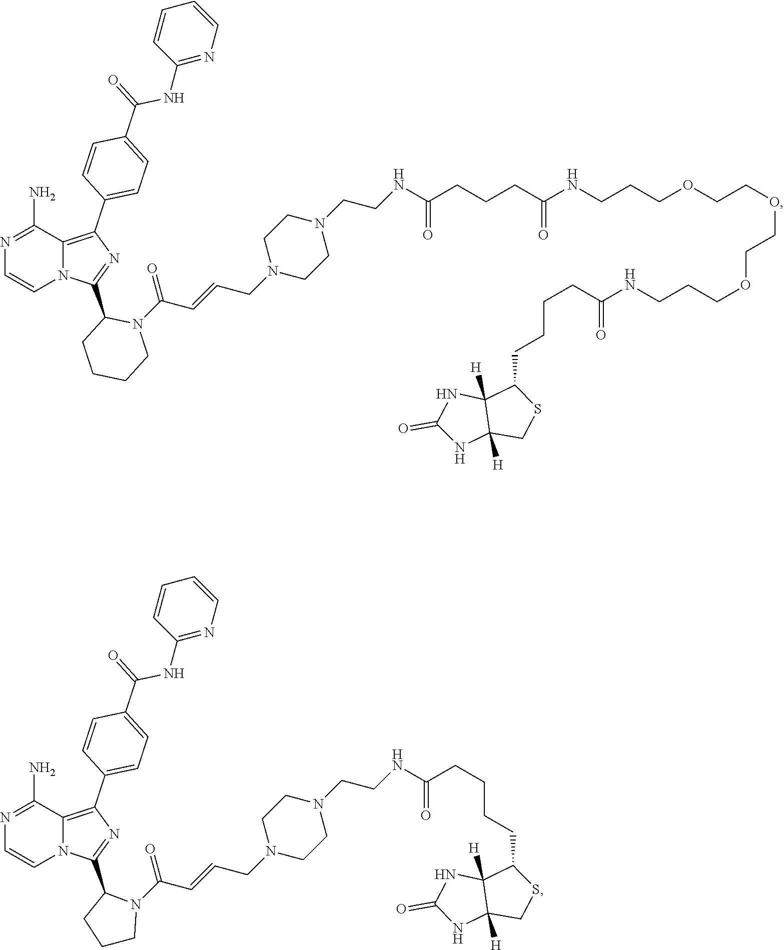 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12] 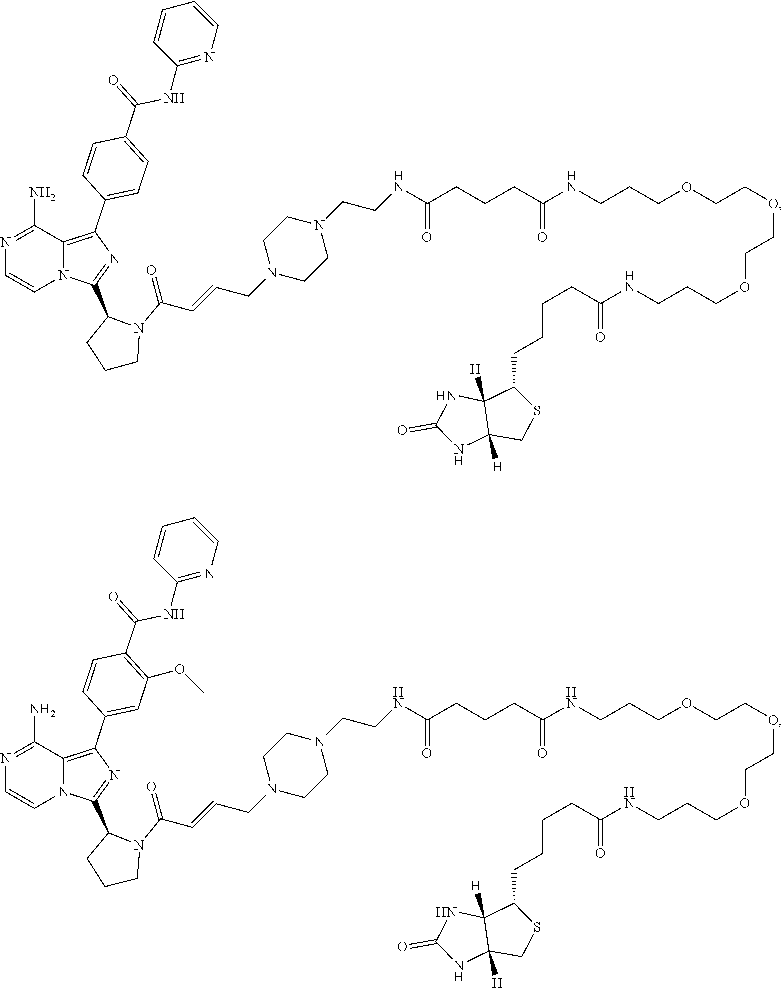 [SMILES: Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1] 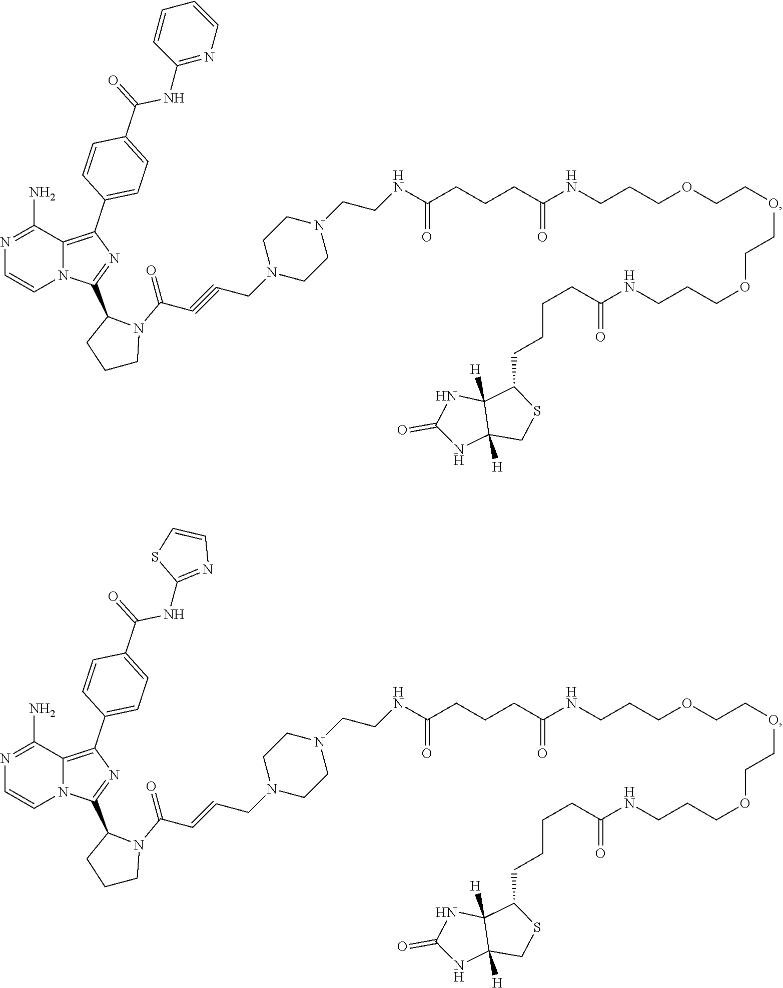 [SMILES: Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]
and salts or complexes thereof.

8. The method of claim 1, wherein the BTK inhibitor is selected from the group consisting of ibrutinib, acalabrutinib, ONO-4059, and pharmaceutically-acceptable salts, esters, prodrugs, cocrystals, solvates, or hydrates thereof.

9. The method of claim 8, wherein the BTK inhibitor is acalabrutinib.

10. The method of claim 1, further comprising the step of adjusting a therapeutic regimen based on the drug target occupancy of BTK.

11. The method of claim 1, wherein the patient is suffering from a BTK-mediated disorder.

12. The method of claim 11, wherein the BTK mediated disorder is selected from the group consisting of chronic lymphocytic leukemia, small lymphocytic leukemia, non-Hodgkin's lymphoma, diffuse large B cell lymphoma, follicular lymphoma, mantle cell lymphoma, Hodgkin's lymphoma, B cell acute lymphoblastic leukemia, Burkitt's lymphoma, Waldenström's macroglobulinemia, multiple myeloma, myelofibrosis, bladder cancer, head and neck cancer, pancreatic cancer, colon cancer, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, head and neck cancer, renal cancer, kidney cancer, liver cancer, prostate cancer, esophageal cancer, testicular cancer, gynecological cancer, thyroid cancer, glioblastoma, esophogeal tumors, hematological neoplasms, acquired immune deficiency syndrome (AIDS)-related lymphoma, Kaposi's sarcoma, viral-induced cancer, non-small-cell lung cancer, small-cell lung cancer, chronic myelocytic leukemia, hepatitis C virus infection, hepatocellular carcinoma, metastatic colon cancer, primary central nervous system lymphoma, ovary tumor, tumor angiogenesis, chronic inflammatory disease, rheumatoid arthritis, atherosclerosis, inflammatory bowel disease, psoriasis, eczema, scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcets disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spoldylitis, Crohn's Disease, lupus, and lupus nephritis.

13. A compound according to: 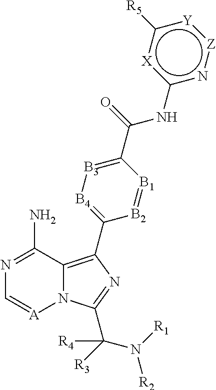
or a salt or complex thereof, wherein:
X is CH or S;
Y is C(R₆);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
L₁ is selected from the group consisting of heterocycloalkyl and heteroalkyl;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

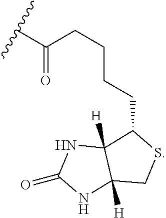 [SMILES: *C(=O)CCCC[C@@H]1SC[C@@H]2NC(=O)N[C@@H]21]

14. The compound of claim 13, wherein L₁ is selected from the group consisting of: 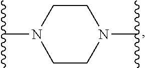 [SMILES: *N1CCN(*)CC1]
—O—, —(C₁₋₅)alkoxy-, and —[(C₁₋₁₀)alkyl]amino-.

15. The compound of claim 13, wherein R₁₂ is:

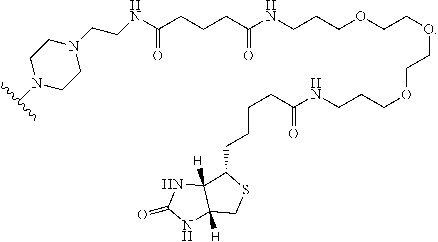 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

16. The compound of claim 13, wherein the compound is selected from the group consisting of: 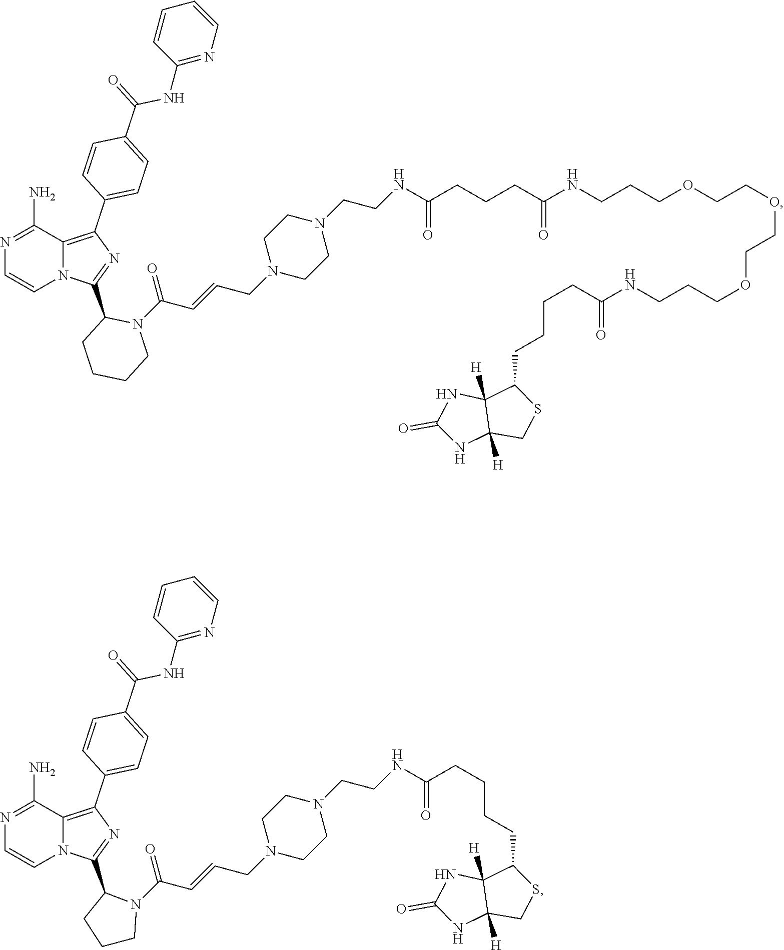 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12] 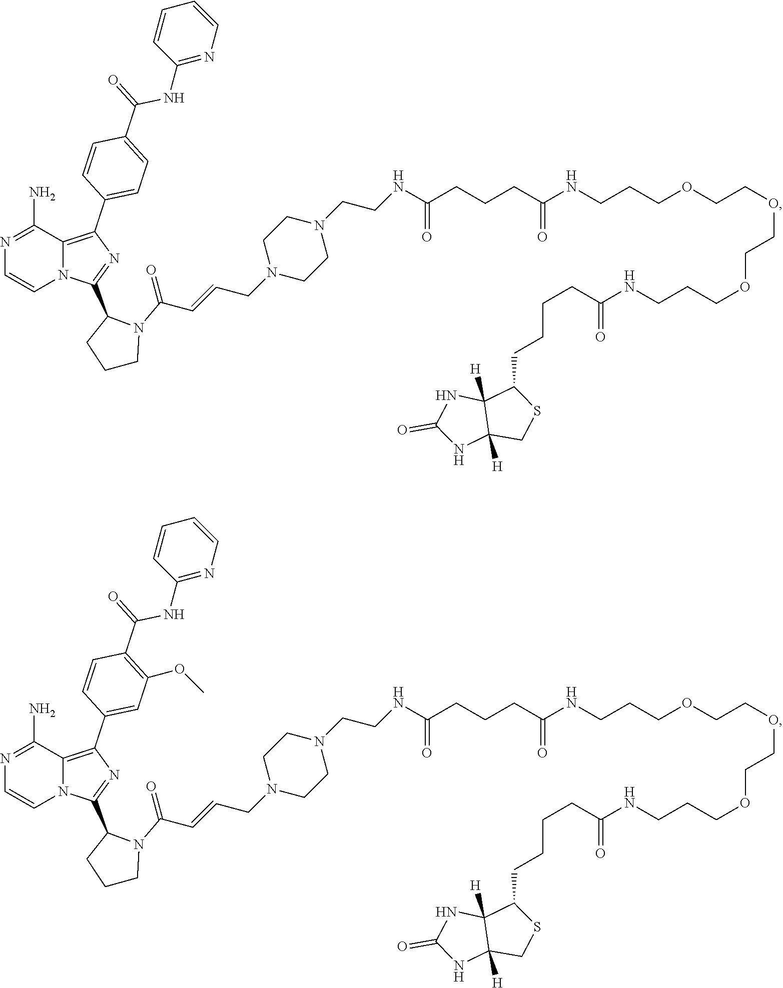 [SMILES: Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1] 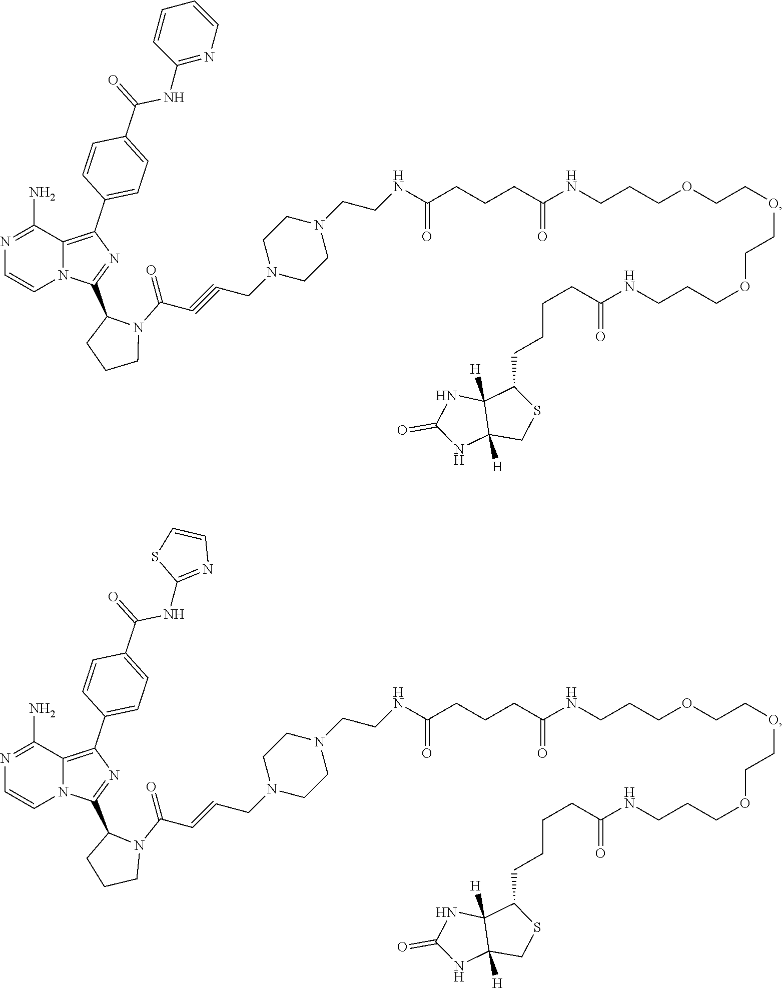 [SMILES: Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]
and salts or complexes thereof.

17. (canceled)

18. A kit for determining drug target occupancy in a patient receiving BTK inhibitor therapy, comprising a BTK probe according to: 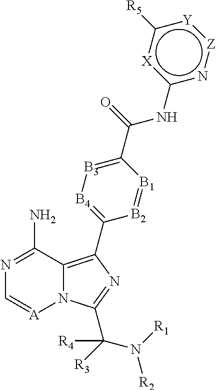
or a salt or complex thereof, wherein:
X is CH or S;
Y is C(R₈);
Z is CH or bond;
A is CH;
B₁ is N or C(R₇);
B₂ is N or C(R₈);
B₃ is N or CH;
B₄ is N or CH;
R₁ is C(═O)R₁₁,
R₂ is (C₁₋₃)alkyl;
R₃ is (C₁₋₃)alkyl;
R₂ and R₃ form a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or any alkyl group of which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
with the proviso that 0 to 2 atoms of B₁, B₂, B₃, and B₄ are N;
R₁₁ is selected from the group consisting of (C₂₋₆)alkenyl-R₁₂ and (C₂₋₆)alkynyl-R₁₂; and
R₁₂ is L₁-L₂-(L₃)ₘ-(L₄-)ₙ-W, wherein:
L₁ is selected from the group consisting of heterocycloalkyl and heteroalkyl;
L₂ is a linear linker group selected from the group consisting of (C₁₋₅)alkylamide, (C₁₋₅)alkoxy, and a bond;
L₃ is a linear linker group selected from the group consisting of (C₁₋₅)alkylamide, (C₁₋₅)alkoxy, and a bond;
L₄ is a linear linker group selected from the group consisting of (C₁₋₅)alkylamide, (C₁₋₅)alkoxy, and a bond;
m is 0 to 5;
n is 0 to 5; and
W is:

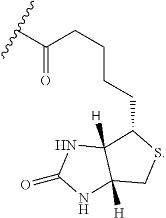 [SMILES: *C(=O)CCCC[C@@H]1SC[C@@H]2NC(=O)N[C@@H]21]

19. The kit of claim 18, wherein L₁ is selected from the group consisting of: 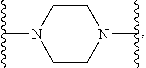 [SMILES: *N1CCN(*)CC1]
—O—, —(C₁₋₅)alkoxy-, and —[(C₁₋₁₀)alkyl]amino-.

20. The kit of any of claim 18 or 19, wherein R₁₂ is:

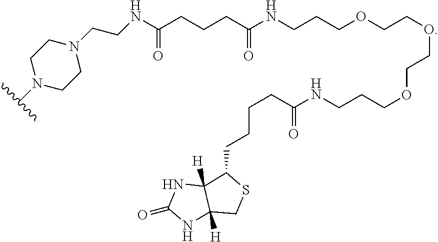 [SMILES: *N1CCN(CCNC(=O)CCCC(=O)NCCCOCCOCCOCCCNC(=O)CCCC[C@@H]2SC[C@@H]3NC(=O)N[C@@H]32)CC1]

21. The kit of claim 18, wherein the BTK probe is a compound selected from the group consisting of: 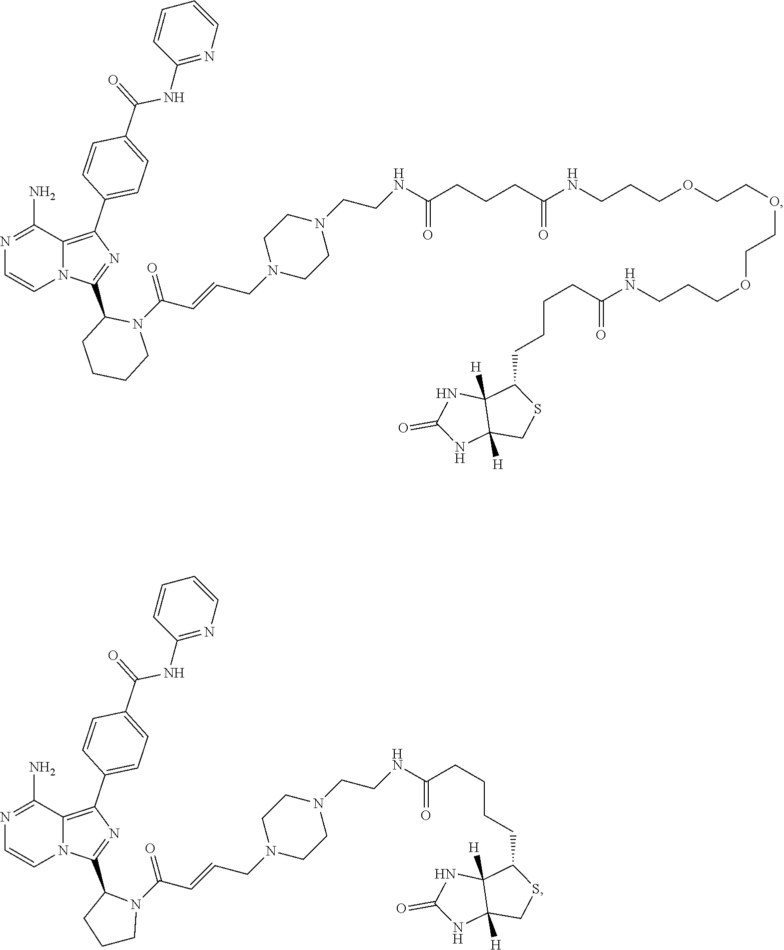 [SMILES: Nc1nccn2c([C@@H]3CCCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC[C@@H]4SC[C@@H]5NC(=O)N[C@@H]54)CC3)nc(-c3ccc(C(=O)Nc4ccccn4)cc3)c12] 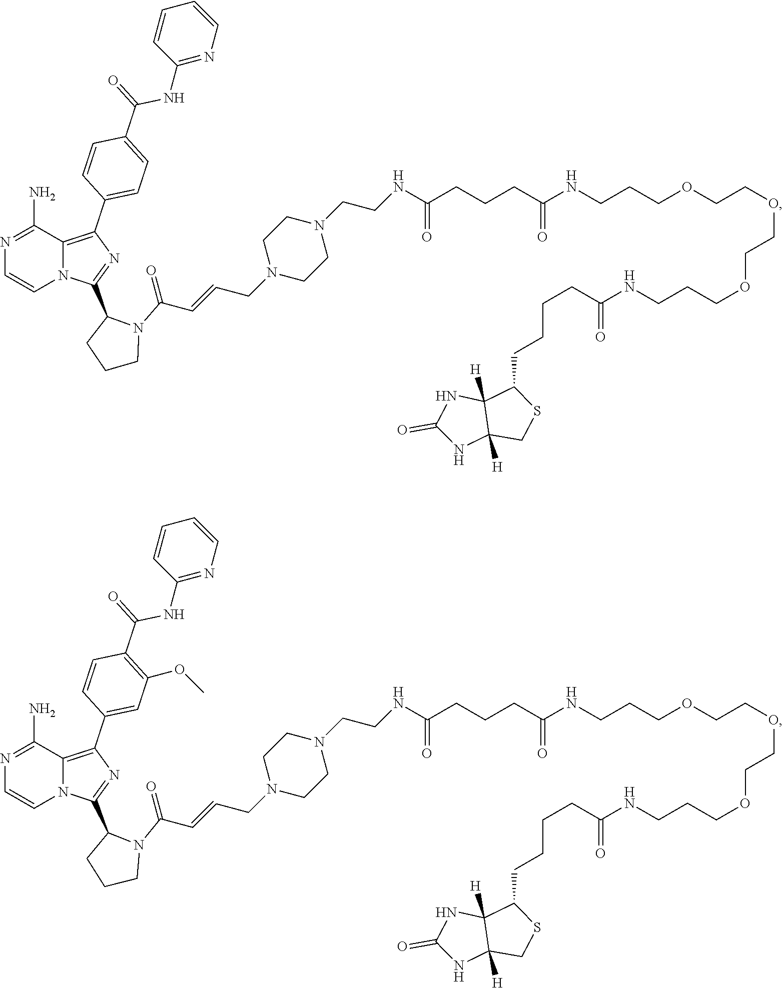 [SMILES: Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; COc1cc(-c2nc([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)n3ccnc(N)c23)ccc1C(=O)Nc1ccccn1] 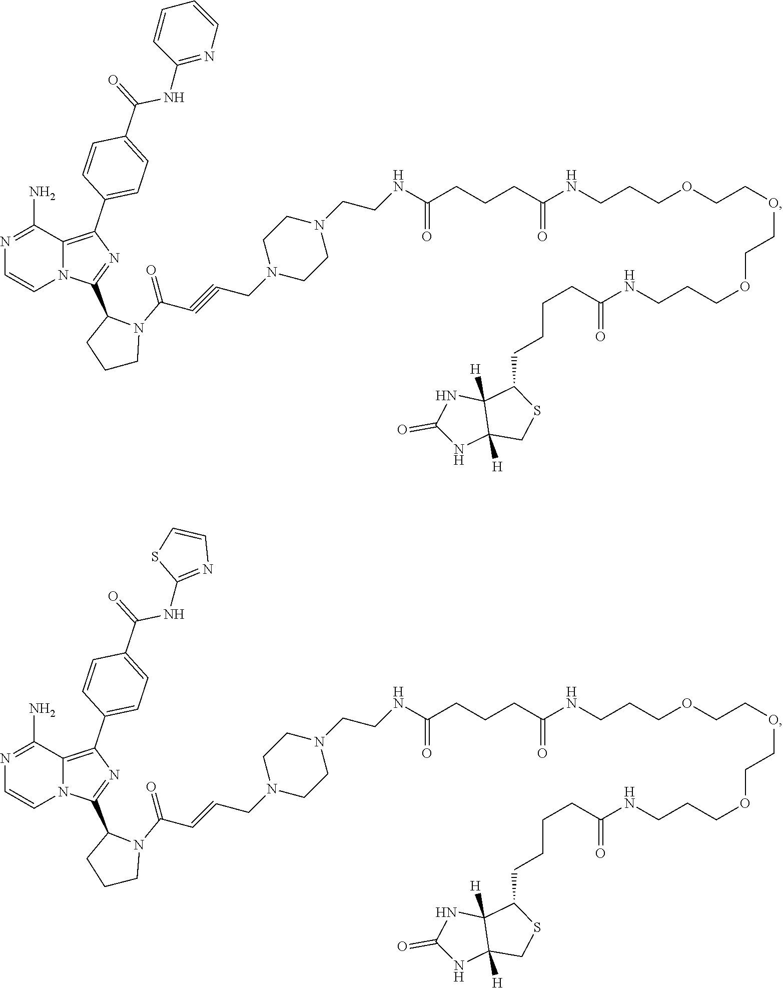 [SMILES: Nc1nccn2c([C@@H]3CCCN3C(=O)C#CCN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4ccccn4)cc3)c12 ; Nc1nccn2c([C@@H]3CCCN3C(=O)/C=C/CN3CCN(CCNC(=O)CCCC(=O)NCCCOCCOCCOCCCNC(=O)CCCC[C@@H]4SC[C@@H]5NC(=O)N[C@@H]54)CC3)nc(-c3ccc(C(=O)Nc4nccs4)cc3)c12]
and salts or complexes thereof.

22. (canceled)

23. (canceled)
